(19) United States
(12) Patent Application Publication
(10) Pub. No.: US 2025/0345364 A1
(43) Pub. Date: Nov. 13, 2025

(54) CISH GENE EDITING OF TUMOR INFILTRATING LYMPHOCYTES AND USES OF SAME IN IMMUNOTHERAPY

(71) Applicants: Iovance Biotherapeutics, Inc., San Carlos, CA (US); CELLECTIS SA, Paris (FR)

(72) Inventors: Krit RITTHIPICHAI, Tampa, FL (US); Alexandre JUILLERAT, Paris (FR); Alex BOYNE, Paris (FR)

(21) Appl. No.: 18/551,586

(22) PCT Filed: Mar. 22, 2022
(86) PCT No.: PCT/US22/21356

§ 371 Date: Sep. 20, 2023

Related U.S. Application Data
(60) Provisional application No. 63/165,066, filed on Mar. 23, 2021.

(51) Int. Cl. A61K 35/17; A61K 40/11; C12N 5/00; C12N 5/0783; C12N 9/22; C12N 15/11; C12N 15/87

(52) U.S. Cl. CPC A61K 35/17; A61K 40/11; C12N 5/0018; C12N 5/0636; C12N 9/22; C12N 15/111; C12N 15/87; C07K 2319/80; C12N 2501/2302; C12N 2501/515; C12N 2510/00

(57) ABSTRACT

The present invention provides improved and/or shortened processes and methods for preparing TILs in order to prepare therapeutic populations of genetically modified TILs with reduced expression of CISH and optionally PD-1 as described herein.

### BACKGROUND OF THE INVENTION

This application claims priority to U.S. Provisional Patent Application No. 63/165,066, filed Mar. 23, 2021, which is incorporated herein by reference in its entirety for all purposes.

### BACKGROUND OF THE INVENTION

Cytokine-inducible SH2-containing protein is a protein that in humans is encoded by the CISH gene. See, Uchida, et al. (1997) Cytogenet Genome Res., 78:209-212. CISH orthologs have been identified in most mammals with sequenced genomes. CISH controls T cell receptor (TCR) signaling, and variations of CISH with certain SNPs are associated with susceptibility to bacteremia, tuberculosis and malaria. See, Khor, et al. (2010) N Engl J Med, 362 (22): 2092-101. The protein encoded by this gene contains a SH2 domain and a SOCS box domain. The protein thus belongs to the cytokine-induced STAT inhibitor (CIS), also known as suppressor of cytokine signaling (SOCS) or STAT-induced STAT inhibitor (SSI), protein family. CIS family members are known to be cytokine-inducible negative regulators of cytokine signaling.

CISH expression can be induced by interleukin-2 (IL-2), IL-3 and granulocyte-macrophage colony-stimulating factor (GM-CSF) in the appropriate cell types. Immunoprecipitation analysis demonstrated that the CISH protein bound stably to the IL-3R beta chain and the EPOR (erythropoietin receptor), but only after ligand binding, suggesting that tyrosine phosphorylation of receptor was required. Over-expression of the CISH protein suppressed cell growth, indicating that CISH had a negative effect on signal transduction. Subsequently, CISH expression was shown to be dependent on STAT5 activation, and several STAT5 binding sites were found in the CISH promoter region. See, Matsumoto, et al. (1997) Blood 89(9):3148-54. Moreover, CISH inhibited EPO-dependent activation of STAT5 and suppressed activity of other STAT5-dependent receptors, indicating that CISH is a feedback modulator for STAT5.

A wide variety of STAT5-dependent receptors induce CISH expression, including (but not limited to) growth hormone (GH), prolactin (PRL), thrombopoietin (TPO), leptin, IL-2, IL-5 and IL-9. See, Bhattacharya, et al. (2001) Am J Respir Cell Mol Biol, 24(3):312-6. CISH has been shown to bind and inhibit signaling from the GH receptor (GHR), the PRL receptor, and IL-2 receptor beta-chain, and to promote internalization and deactivation of the GHR. See, Ram, et al. (1999) Biol Chem, 274(50):35553-61; Endo, et al. (2003) J Biochem 133(1):109-13; Aman, et al. (1999) J Biol Chem 274(42):30266-72; Landsman, et al. (2005) J Biol Chem 280(45):37471-80. Expression of CISH mRNA is found in a number of tissues (liver, kidney, heart stomach, lung, ovary and skeletal muscle). See, Palmer, et al. (2009) 30(12):592-602; Anderson, et al. (2009) 138(3):537-44; Clasen, et al. (2013) JLipidRes 54(7):1988-97. In spite of its apparent involvement in the signaling apparatus of a large number of important cytokines and growth factors, CISH knockout mice have minimal defects (except for subtle changes in the immune response). See, Palmer, et al. (2009) Trends Immunol 30(12):592-602; Trengove, et al. (2013) Am J Cin Exp Immunol 2(1): 1-29. This may be due to compensatory activity of the other SOCS family proteins. An effect of CISH on the biology of putative target genes was observed in transgenic mice constitutively expressing CISH driven from the beta-actin promoter. Those mice had reduced body weight, defects in mammary gland development and reduced numbers of gamma/delta T cells, natural killer (NK) cells and NKT cells, a phenotype that resembled StatSa and/or StatSb deficient mice. See, Matsumoto, et al. (1999) Mol Cell Biol 19(9):6396-407.

CISH potentially influences signaling by many cytokines and growth factors, and CISH activity and variants have been found to be associated with infectious disease and cancer. Several studies have shown increased susceptibility to various infectious agents in subjects carrying certain CISH polymorphisms, including malaria, leptospirosis, hepatitis B virus and tuberculosis. See, Khor, et al. (2010) N Engl J Med, 362(22):2092-101; Esteves, et al. (2014) PLoS One, 9(9):e108534; Hu, et al. (2014) PLoS One, 9(6):e100826; Tong, et al. (2012) Immunogenetics, 64(4):261-5; Ji, et al. (2014) Infect Genet Evol, 28:240-4; Sun, et al. (2014) PLoS, 9(3):e92020. One risk allele common to all studies (rs414171, -292 from the start of transcription) displayed lower levels of CISH expression in peripheral blood mononuclear cells compared to the alternate allele. See, Khor and Sun, supra. Expression levels of CISH were elevated in breast carcinomas and cancer cell lines compared to normal tissues, leading to speculation that CISH may contribute to tumorigenesis by its ability to activate the extracellular-signal-regulated kinase (ERK). Raccurt, et al. (2003) Br. J Cancer, 89(3):524-32. CISH variants are also associated with milk production traits in dairy cattle. See, Arun, et al. (2015) Front Genet, 6:342.

Engineered nucleases including TALENs, are designed to specifically bind to target DNA sites have the ability to regulate gene expression of endogenous genes and can be useful in genome engineering, gene therapy and treatment of disorders such as cancer and inflammation. See, e.g., U.S. Pat. Nos. 9,877,988; 9,394,545; 9,150,847; 9,206,404; 9,045,763; 9,005,973; 8,956,828; 8,936,936; 8,945,868; 8,871,905; 8,586,526; 8,563,314; 8,329,986; 8,399,218; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publication Nos. 2003/0232410; 2005/0208489; 2005/0026157; 2005/0064474; 2006/0063231; 2008/0159996; 2010/0218264; 2012/0017290; 2011/0265198; 2013/0137104; 2013/0122591; 2013/0177983; 2013/0177960; and 2015/0056705, the disclosures of which are incorporated by reference in their entireties for all purposes. Further, targeted nucleases are being developed based on the Argonaute system (e.g., from T. thermophilus, known as ‘TtAgo’, see Swarts, et al. (2014) Nature 507(7491): 258-261), which also may have the potential for uses in genome editing and gene therapy.

TALE-mediated gene therapy can be used to genetically engineer a cell to have one or more inactivated genes and/or to cause that cell to express a product not previously being produced in that cell (e.g., via transgene insertion and/or via correction of an endogenous sequence). Clinical trials using these nucleases have shown that these molecules are capable of treating various conditions, including cancers, HIV and/or blood disorders (such as hemoglobinopathies and/or hemophilias). See, e.g., Yu, et al. (2006) FASEB J. 20:479-481; Tebas, et al. (2014) New Eng J Med 370(10):901. Thus, these approaches can be used for the treatment of diseases. However, there remains a need for additional methods and compositions for CISH TALEN mediated gene inactivation/deletion for treatment and/or prevention of cancer, inflammatory disorders, and other diseases in which CISH modulation is desired.

Treatment of bulky, refractory cancers using adoptive transfer of tumor infiltrating lymphocytes (TILs) represents a powerful approach to therapy for patients with poor prognoses. Gattinoni, et al., Nat. Rev. Immunol. 2006, 6, 383-393. There is an urgent need to provide manufacturing processes and therapies for genetically modified TILs based on such processes that are appropriate for commercial scale manufacturing and regulatory approval for use in human patients at multiple clinical centers. In particular, there remains a need in the art for additional methods and compositions for CISH and/or PD-1 gene inactivation/deletion in combination with TIL based therapies for treatment and/or prevention of cancer, inflammatory disorders, and other diseases in which CISH and/or PD-1 modulation is desired and the present invention meets that need.

### BRIEF SUMMARY OF THE INVENTION

The present invention provides a method of preparing genetically modified tumor infiltrating lymphocytes (TILs) comprising reduced expression of CISH and optionally PD-1, the method comprising:
(a) introducing into the TILs nucleic acid(s) encoding one or more first Transcription activator-like effector nucleases (TALE-nuclease) able to selectively inactivate by DNA cleavage a gene encoding CISH, wherein the one or more first TALE-nucleases comprise a TALE-nuclease that is directed against the nucleic acid sequence of SEQ ID NO: 175, and optionally introducing one or more second TALE-nucleases able to selectively inactivate by DNA cleavage a gene encoding PD-1; and
(b) expanding the TILs.

In some embodiments, the method comprises introducing into the TILs nucleic acid(s) encoding the one or more first TALE-nucleases comprises an electroporation step.

In some embodiments, the nucleic acid(s) encoding the one or more first TALE-nucleases are RNA and the RNA are introduced into the TILs by electroporation.

In some embodiments, the method further comprises prior to the introducing step, a step of activating TILs by culturing the TILs in a cell culture medium in the presence of OKT-3 for about 1-3 days.

In some embodiments, the method further comprises after the introducing step and before the expanding step, a step of resting the TILs in a cell culture medium comprising IL-2 for about 1 day.

T In some embodiments, the method further comprises prior to the introducing step, a step of cryopreserving the TILs followed by thawing and culturing the TILs in a cell culture medium comprising IL-2 for about 1-3 days.

In some embodiments, the IL-2 in the resting step is at a concentration of about 3000 IU/ml.

In some embodiments, the one or more first TALE-nucleases are each constituted by a first half-TALE nuclease and a second half-TALE nuclease.

In some embodiments, the first half-TALE nuclease is a first fusion protein constituted by a first TALE nucleic acid binding domain fused to a first nuclease catalytic domain and the second half-TALE nuclease is a second fusion protein constituted by a second TALE nucleic acid binding domain fused to a second nuclease catalytic domain.

In some embodiments, the first TALE nucleic acid binding domain has a first amino acid sequence and the second TALE nucleic acid binding domain has a second amino acid sequence, and wherein the first amino acid sequence is different from the second amino acid sequence.

In some embodiments, the first nuclease catalytic domain has a first amino acid sequence and the second nuclease catalytic domain has a second amino acid sequence, and wherein the first amino acid sequence is the same as the second amino acid sequence.

In some embodiments, the first nuclease catalytic domain and the second nuclease catalytic domain both have the amino acid sequence of Fok-I.

In some embodiments, the first half-TALE nuclease and the second half-TALE nuclease are capable of forming a heterodimeric DNA cleavage complex to effect DNA cleavage at the target site in the gene encoding CISH, and wherein the target site in the gene encoding CISH comprises the nucleic acid sequence of SEQ ID NO: 175.

In some embodiments, the first half-TALE nuclease recognizes a first half-target located at a first location in the target site in the gene encoding CISH and the second half-TALE nuclease recognizes a second half-target located in a second location in the target site in the gene encoding CISH that does not overlap with the first location.

In some embodiments, the TALE nuclease comprises an amino acid sequence having at least 70%, 75%, 80%, 85%, 87.5%, 90%, 92.5%, 95%, 97.5%, 98%, or 99% sequence identity with an amino acid sequence selected from the group consisting of SEQ ID NO: 165 and SEQ ID NO: 167.

In some embodiments, the TALE-nuclease comprises a sequence selected from the group consisting of SEQ ID NO: 165 and SEQ ID NO: 167.

In some embodiments, the first half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 165 and the second half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 167.

In some embodiments, the first half-TALE-nuclease comprises the amino acid sequence of SEQ ID NO: 165 and the second half-TALE-nuclease comprises the amino acid sequence of SEQ ID NO: 167.

In some embodiments, the expanded TILs comprise sufficient TILs for administering a therapeutically effective dosage of the TILs to a subject in need thereof.

In some embodiments, the therapeutically effective dosage of the expanded TILs comprises from about 1×10⁹ to about 9×10¹⁰ TILs.

The present invention also provides a population of expanded tumor infiltrating lymphocytes (TILs) comprising reduced expression of CISH and optionally PD-1, the population of expanded TILs being obtainable by the method of any of the claims 1 to 20.

The present invention also provides a Transcription activator-like effector nuclease (TALE-nuclease) that recognizes and effects DNA cleavage at a target site in a gene encoding CISH, wherein the TALE-nuclease comprises an amino acid sequence having at least 70%, 75%, 80%, 85%, 87.5%, 90%, 92.5%, 95%, 97.5%, 98%, or 99% sequence identity with an amino acid sequence selected from the group consisting of SEQ ID NO: 165 and SEQ ID NO: 167.

In some embodiments, the TALE-nuclease comprises a sequence selected from the group consisting of SEQ ID NO: 165 and SEQ ID NO: 167.

In some embodiments, the TALE-nuclease is constituted by a first half-TALE nuclease and a second half-TALE nuclease, and wherein the first half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 165 and the second half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 167.

In some embodiments, the first half-TALE-nuclease comprises the amino acid sequence of SEQ ID NO: 165 and the second half-TALE-nuclease comprises the amino acid sequence of SEQ ID NO: 167.

In some embodiments, the first half-TALE nuclease is a first fusion protein constituted by a first TALE nucleic acid binding domain fused to a first nuclease catalytic domain and the second half-TALE nuclease is a second fusion protein constituted by a second TALE nucleic acid binding domain fused to a second nuclease catalytic domain.

In some embodiments, the first TALE nucleic acid binding domain has a first amino acid sequence and the second TALE nucleic acid binding domain has a second amino acid sequence, and wherein the first amino acid sequence is different from the second amino acid sequence.

In some embodiments, the first nuclease catalytic domain has a first amino acid sequence and the second nuclease catalytic domain has a second amino acid sequence, and wherein the first amino acid sequence is the same as the second amino acid sequence.

In some embodiments, the first nuclease catalytic domain and the second nuclease catalytic domain both have the amino acid sequence of Fok-I.

In some embodiments, the first half-TALE nuclease and the second half-TALE nuclease are capable of forming a heterodimeric DNA cleavage complex to effect DNA cleavage at a target site in the gene encoding CISH, and wherein the target site comprises the nucleic acid sequence of SEQ ID NO: 175.

In some embodiments, the first half-TALE nuclease recognizes a first half-target located at a first location in the target site in the gene encoding CISH and the second half-TALE nuclease recognizes a second half-target located in a second location in the target site in the gene encoding CISH that does not overlap with the first location.

The present invention provides a method for expanding genetically modified tumor infiltrating lymphocytes (TILs) into a therapeutic population of TILs comprising reduced expression of CISH and optionally PD-1, the method comprising:
(a) obtaining and/or receiving a first population of TILs from a tumor resected from a subject by processing a tumor sample obtained from the subject into multiple tumor fragments;
(b) adding the first population of TILs into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) introducing into the TILs nucleic acid(s) encoding one or more first Transcription activator-like effector nucleases (TALE-nuclease) able to selectively inactivate by DNA cleavage a gene encoding CISH, wherein the one or more first TALE-nucleases comprise a TALE-nuclease that is directed against a target site in the gene encoding CISH, wherein the target site comprises the nucleic acid sequence of SEQ ID NO: 175, and optionally introducing into the TILs nucleic acid(s) encoding one or more second TALE-nucleases able to selectively inactivate by DNA cleavage a gene encoding PD-1;
(e) performing a second expansion by culturing the TILs obtained from step (d) in a cell culture medium comprising IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area; and
(f) harvesting the therapeutic population of TILs obtained from step (e), wherein the transition from step (e) to step (f) occurs without opening the system;
(g) transferring the harvested TIL population from step (f) to an infusion bag, wherein the transfer from step (f) to (g) occurs without opening the system.

The present invention provides a method for expanding genetically modified tumor infiltrating lymphocytes (TILs) into a therapeutic population of TILs comprising reduced expression of CISH and optionally PD-1, the method comprising:
(a) obtaining a first population of TILs from a tumor resected from a subject by processing a tumor sample obtained from the subject into multiple tumor fragments;
(b) adding the tumor fragments into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) introducing into the TILs nucleic acid(s) encoding one or more first Transcription activator-like effector nucleases (TALE-nuclease) able to selectively inactivate by DNA cleavage a gene encoding CISH, wherein the one or more first TALE-nucleases comprise a TALE-nuclease that is directed against a target site in the gene encoding CISH, wherein the target site comprises the nucleic acid sequence of SEQ ID NO: 175, and optionally introducing into the TILs nucleic acid(s) encoding one or more second TALE-nucleases able to selectively inactivate by DNA cleavage a gene encoding PD-1;
(e) performing a second expansion by culturing the TILs obtained from step (d) in a cell culture medium comprising IL-2, OKT-3, and antigen presenting cells (APCs), to produce a third population of TILs, wherein the second expansion is performed for about 7-11 days to obtain the third population of TILs, wherein the third population of TILs is a therapeutic population of TILs, wherein the second expansion is performed in a closed container providing a second gas-permeable surface area;
(f) harvesting the therapeutic population of TILs obtained from step (e), wherein the transition from step (e) to step (f) occurs without opening the system; and
(g) transferring the harvested therapeutic TIL population from step (f) to an infusion bag, wherein the transfer from step (f) to (g) occurs without opening the system.

The present invention provides a method for expanding genetically modified tumor infiltrating lymphocytes (TILs) into a therapeutic population of TILs comprising reduced expression of CISH and optionally PD-1, the method comprising:
(a) obtaining and/or receiving a first population of TILs from surgical resection, needle biopsy, core biopsy, small biopsy, or other means for obtaining a sample that contains a mixture of tumor and TIL cells from a melanoma in the subject,
(b) adding the first population of TILs into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) introducing into the TILs nucleic acid(s) encoding one or more first Transcription activator-like effector nucleases (TALE-nuclease) able to selectively inactivate by DNA cleavage a gene encoding CISH, wherein the one or more first TALE-nucleases comprise a TALE-nuclease that is directed against a target site in the gene encoding CISH, wherein the target site comprises the nucleic acid sequence of SEQ ID NO: 175, and optionally introducing one or more second TALE-nucleases able to selectively inactivate by DNA cleavage a gene encoding PD-1;
(e) performing a second expansion by culturing the TILs obtained from step (d) in a cell culture medium comprising IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area;
(f) harvesting the therapeutic population of TILs obtained from step (e), wherein the transition from step (e) to step (f) occurs without opening the system; and
(g) transferring the harvested therapeutic TIL population from step (f) to an infusion bag, wherein the transfer from step (f) to (g) occurs without opening the system.

The present invention provides a method for expanding genetically modified tumor infiltrating lymphocytes (TILs) into a therapeutic population of TILs comprising reduced expression of CISH and optionally PD-1, the method comprising:
(a) resecting a tumor from the subject, the tumor comprising a first population of TILs, optionally from surgical resection, needle biopsy, core biopsy, small biopsy, or other means for obtaining a sample that contains a mixture of tumor and TIL cells;
(b) adding the tumor fragments into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) introducing into the TILs nucleic acid(s) encoding one or more first Transcription activator-like effector nucleases (TALE-nuclease) able to selectively inactivate by DNA cleavage a gene encoding CISH, wherein the one or more first TALE-nucleases comprise a TALE-nuclease that is directed against a target site in the gene encoding CISH, wherein the target site comprises the nucleic acid sequence of SEQ ID NO: 175, and optionally introducing one or more second TALE-nucleases able to selectively inactivate by DNA cleavage a gene encoding PD-1;
(e) performing a second expansion by culturing the TILs obtained from step (d) in a cell culture medium comprising IL-2, OKT-3, and antigen presenting cells (APCs), to produce a third population of TILs, wherein the second expansion is performed for about 7-11 days to obtain the third population of TILs, wherein the third population of TILs is a therapeutic population of TILs, wherein the second expansion is performed in a closed container providing a second gas-permeable surface area;
(f) harvesting the third population of TILs obtained from step (e), wherein the transition from step (e) to step (f) occurs without opening the system; and
(g) transferring the harvested third TIL population from step (f) to an infusion bag, wherein the transfer from step (f) to (g) occurs without opening the system.

The present invention provides a method for expanding genetically modified tumor infiltrating lymphocytes (TILs) into a therapeutic population of TILs comprising reduced expression of CISH and optionally PD-1, the method comprising:
(a) obtaining a first population of TILs from a tumor resected from a subject by processing a tumor sample obtained from the subject into multiple tumor fragments;
(b) adding the tumor fragments into a closed system;
(c) performing a first expansion by culturing the first population of TILs in a cell culture medium comprising IL-2, and optionally OKT-3, to produce a second population of TILs, wherein the first expansion is performed in a closed container providing a first gas-permeable surface area, wherein the first expansion is performed for about 3-14 days to obtain the second population of TILs, wherein the transition from step (b) to step (c) occurs without opening the system;
(d) introducing into the TILs nucleic acid(s) encoding one or more first Transcription activator-like effector nucleases (TALE-nuclease) able to selectively inactivate by DNA cleavage a gene encoding CISH, wherein the one or more first TALE-nucleases comprise a TALE-nuclease that is directed against a target site in the gene encoding CISH, wherein the target site comprises the nucleic acid sequence of SEQ ID NO: 175, and optionally introducing one or more second TALE-nucleases able to selectively inactivate by DNA cleavage a gene encoding PD-1;
(d) performing a second expansion by culturing the TILs obtained from step (d) in a cell culture medium comprising IL-2, optionally OKT-3, and antigen presenting cells (APCs), to produce a third population of TILs, wherein the second expansion is performed for about 4-6 days to obtain the third population of TILs, wherein the third population of TILs is a therapeutic population of TILs, wherein the second expansion is performed in a closed container providing a second gas-permeable surface area;
(e) dividing the third population of TILs into a first plurality of 2-5 subpopulations of TILs, wherein at least 1.0×10⁹ TILs are present in each subpopulation, wherein the transition from step (d) to step (e) occurs without opening the system;
(f) performing a third expansion of the first plurality of subpopulations of TILs by supplementing the cell culture medium of each subpopulation of TILs with additional IL-2, optionally OKT-3, to produce a second plurality of subpopulations of TILs, wherein the third expansion is performed for about 5-7 days, wherein the third expansion for each subpopulation is performed in a closed container providing a third gas-permeable surface area, and wherein the transition from step (e) to step (f) occurs without opening the system; and
(g) harvesting the second plurality of subpopulations of TILs obtained from step (f); and
(h) transferring the harvested subpopulations of TILs from step (g) to one or more infusion bags, wherein the transition from step (g) to (h).

In some embodiments, the method further comprises a step of cryopreserving the harvested TILs using a cryopreservation process.

In some embodiments, the nucleic acid(s) encoding the one or more first TALE-nucleases are RNA.

In some embodiments of the method, introducing the nucleic acid(s) encoding the one or more first TALE-nucleases are introduced into the TILs by electroporation.

In some embodiments, the method further comprises prior to the introducing step, a step of activating TILs by culturing the TILs in a cell culture medium in the presence of OKT-3 for about 1-3 days.

In some embodiments, the OKT-3 is at a concentration of about 300 ng/ml.

In some embodiments, the method further comprises after the introducing step and before the second expansion step, a step of resting the TILs in a cell culture medium comprising IL-2 for about 1 day.

In some embodiments, the resting step is at a concentration of about 3000 IU/ml.

In some embodiments, the method further comprises cryopreserving the TILs followed by thawing and culturing the TILs in a cell culture medium comprising IL-2 for about 1-3 days.

In some embodiments, steps (a) through (g) are performed in about 13 days to about 29 days, optionally about 15 days, about 16 days, about 17 days, about 18 days, about 19 days, about 20 days, about 21 days, about 22 days, about 23 days, about 24 days, or about 25 days.

In some embodiments, the nucleic acid(s) encoding the one or more first TALE-nucleases are RNA, and the RNA are introduced into the TILs by electroporation.

In some embodiments, the one or more first TALE-nucleases are each constituted by a first half-TALE nuclease and a second half-TALE nuclease.

In some embodiments, the first half-TALE nuclease is a first fusion protein constituted by a first TALE nucleic acid binding domain fused to a first nuclease catalytic domain and the second half-TALE nuclease is a second fusion protein constituted by a second TALE nucleic acid binding domain fused to a second nuclease catalytic domain.

In some embodiments, the first TALE nucleic acid binding domain has a first amino acid sequence and the second TALE nucleic acid binding domain has a second amino acid sequence, and wherein the first amino acid sequence is different from the second amino acid sequence.

In some embodiments, the first nuclease catalytic domain has a first amino acid sequence and the second nuclease catalytic domain has a second amino acid sequence, and wherein the first amino acid sequence is the same as the second amino acid sequence.

In some embodiments, the first nuclease catalytic domain and the second nuclease catalytic domain both have the amino acid sequence of Fok-I.

In some embodiments, the first half-TALE nuclease and the second half-TALE nuclease are capable of forming a heterodimeric DNA cleavage complex to effect DNA cleavage at the target site.

In some embodiments, the first half-TALE nuclease recognizes a first half-target located at a first location in the target site and the second half-TALE nuclease recognizes a second half-target located in a second location in the target site that does not overlap with the first location.

In some embodiments, the TALE-nuclease comprises an amino acid sequence having at least 70%, 75%, 80%, 85%, 87.5%, 90%, 92.5%, 95%, 97.5%, 98%, or 99% sequence identity with an amino acid sequence selected from the group consisting of SEQ ID NO: 165 and SEQ ID NO: 167.

In some embodiments, the TALE-nuclease comprises a sequence selected from the group consisting of SEQ ID NO: 165 and SEQ ID NO: 167.

In some embodiments, the first half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 165 and the second half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 167.

In some embodiments, the first half-TALE-nuclease comprises the amino acid sequence of SEQ ID NO: 165 and the second half-TALE-nuclease comprises the amino acid sequence of SEQ ID NO: 167.

In some embodiments, the TILs harvested comprises sufficient TILs for administering a therapeutically effective dosage of the TILs to a subject in need thereof.

In some embodiments, the therapeutically effective dosage of the TILs comprises from about 1×10⁹ to about 9×10¹⁰ TILs.

In some embodiments, the APCs comprise peripheral blood mononuclear cells (PBMCs).

In some embodiments, the PBMCs are supplemented at a ratio of about 1:25 TIL:PBMCs.

In some embodiments, the therapeutic population of TILs provides for increased efficacy, increased interferon-gamma (IFN-γ) production, increased polyclonality, increased average IP-10, and/or increased average MCP-1 when administered to the subject.

In some embodiments, the IL-2 is present at an initial concentration of between 1000 IU/mL and 6000 IU/mL in the cell culture medium in the first expansion.

In some embodiments, the second expansion step, the IL-2 is present at an initial concentration of between 1000 IU/mL and 6000 IU/mL and the OKT-3 antibody is present at an initial concentration of about 30 ng/mL.

In some embodiments, in the second and/or third expansion step, the IL-2 is present at an initial concentration of between 1000 IU/mL and 6000 IU/mL, and optionally, the OKT-3 antibody is present at an initial concentration of about 30 ng/mL.

In some embodiments, the first expansion is performed using a gas permeable container.

In some embodiments, the second expansion is performed using a gas permeable container.

In some embodiments, the second and/or third expansion is performed using a gas permeable container.

In some embodiments, the first cell culture medium further comprises a cytokine selected from the group consisting of IL-4, IL-7, IL-15, IL-21, and combinations thereof.

In some embodiments, the second cell culture medium further comprises a cytokine selected from the group consisting of IL-4, IL-7, IL-15, IL-21, and combinations thereof.

In some embodiments, the cell culture medium in step (d) and/or (f) further comprises a cytokine selected from the group consisting of IL-4, IL-7, IL-15, IL-21, and combinations thereof.

In some embodiments, the cell culture medium of the second expansion further comprises a cytokine selected from the group consisting of IL-4, IL-7, IL-15, IL-21, and combinations thereof.

In some embodiments, the first expansion in step (c) and/or the second expansion in step (e) are individually performed within a period of 11 days.

The present invention provides a population of or composition comprising genetically modified tumor infiltrating lymphocytes (TILs) comprising reduced expression of CISH and/or PD-1, the population of or composition comprising TILs being obtainable by the method of any of the claims 1 to 20 and 32 to 73.

The present invention provides a method of treating cancer in a subject in need thereof, the method comprising administering to the subject a therapeutic population of genetically modified tumor infiltrating lymphocytes (TILs) comprising reduced expression of CISH and/or CISH and PD-1, wherein the therapeutic population of genetically modified TILs is obtainable by the method of any of the claims 1 to 20 and 32 to 73.

In some embodiments, the cancer is selected from the group consisting of melanoma (including metastatic melanoma), ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), renal cancer, and renal cell carcinoma.

### BRIEF DESCRIPTION OF THE DRAWINGS

FIG. 1: Assessment of Double KO in TIL.

FIG. 2: The efficiency of single and double CISH KO.

FIG. 3: PD-1 KO efficiency in double CISH/PD-1 KO TIL.

FIG. 4: Fold expansion in CISH KO TIL decreased relative to control.

FIG. 5: T-cell Lineage and Memory Subset in CISH KO TIL.

FIG. 6: CISH: Differentiation and Activation/Exhaustion in CISH KO TIL.

FIG. 7: Shows an exemplary processes for expanding the genetically modified TILs by introducing into the TILs nucleic acids encoding one or more TALE-nucleases directed against a target sequence in the CISH gene, which target sequence comprises the nucleic acid sequence of SEQ ID NO: 175.

### BRIEF DESCRIPTION OF THE SEQUENCE LISTING

SEQ ID NO: 1 is the amino acid sequence of the heavy chain of muromonab.

SEQ ID NO: 2 is the amino acid sequence of the light chain of muromonab.

SEQ ID NO: 3 is the amino acid sequence of a recombinant human IL-2 protein.

SEQ ID NO: 4 is the amino acid sequence of aldesleukin.

SEQ ID NO: 5 is the amino acid sequence of a recombinant human IL-4 protein.

SEQ ID NO: 6 is the amino acid sequence of a recombinant human IL-7 protein.

SEQ ID NO: 7 is the amino acid sequence of a recombinant human IL-15 protein.

SEQ ID NO: 8 is the amino acid sequence of a recombinant human IL-21 protein.

SEQ ID NO: 9-126 are currently not assigned.

SEQ ID NO: 127 is a target PD-1 sequence.

SEQ ID NO: 128 is a target PD-1 sequence.

SEQ ID NO: 129 is a repeat PD-1 left repeat sequence.

SEQ ID NO: 130 is a repeat PD-1 right repeat sequence.

SEQ ID NO: 131 is a repeat PD-1 left repeat sequence.

SEQ ID NO: 132 is a repeat PD-1 right repeat sequence.

SEQ ID NO: 133 is a PD-1 left TALEN nuclease sequence.

SEQ ID NO: 134 is a PD-1 right TALEN nuclease sequence.

SEQ ID NO: 135 is a PD-1 left TALEN nuclease sequence.

SEQ ID NO: 136 is a PD-1 right TALEN nuclease sequence.

SEQ ID NO: 137 is the IL-2 sequence.

SEQ ID NO: 138 is an IL-2 mutein sequence.

SEQ ID NO: 139 is an IL-2 mutein sequence.

SEQ ID NO: 140 is the HCDR1_IL-2 for IgG.IL2R67A.H1.

SEQ ID NO: 141 is the HCDR2 for IgG.IL2R67A.H1.

SEQ ID NO: 142 is the HCDR3 for IgG.IL2R67A.H1.

SEQ ID NO: 143 is the HCDR1_IL-2 kabat for IgG.IL2R67A.H1.

SEQ ID NO: 144 is the HCDR2 kabat for IgG.IL2R67A.H1.

SEQ ID NO: 145 is the HCDR3 kabat for IgG.IL2R67A.H1.

SEQ ID NO: 146 is the HCDR1_IL-2 clothia for IgG.IL2R67A.H1.

SEQ ID NO: 147 is the HCDR2 clothia for IgG.IL2R67A.H1.

SEQ ID NO: 148 is the HCDR3 clothia for IgG.IL2R67A.H1.

SEQ ID NO: 149 is the HCDR1_IL-2 IMGT for IgG.IL2R67A.H1.

SEQ ID NO: 150 is the HCDR2 IMGT for IgG.IL2R67A.H1.

SEQ ID NO: 151 is the HCDR3 IMGT for IgG.IL2R67A.H1.

SEQ ID NO: 152 is the VH chain for IgG.IL2R67A.H1.

SEQ ID NO: 153 is the heavy chain for IgG.IL2R67A.H1.

SEQ ID NO: 154 is the LCDR1 kabat for IgG.IL2R67A.H1.

SEQ ID NO: 155 is the LCDR2 kabat for IgG.IL2R67A.H1.

SEQ ID NO: 156 is the LCDR3 kabat for IgG.IL2R67A.H1.

SEQ ID NO: 157 is the LCDR1 chothia for IgG.IL2R67A.H1.

SEQ ID NO: 158 is the LCDR2 chothia for IgG.IL2R67A.H1.

SEQ ID NO: 159 is the LCDR3 chothia for IgG.IL2R67A.H1.

SEQ ID NO: 160 is the VL chain.

SEQ ID NO: 161 is the light chain.

SEQ ID NO: 162 is the light chain.

SEQ ID NO: 163 is the light chain.

SEQ ID NO: 164 is the nucleotide sequence for a left CISH KO TALE-nuclease.

SEQ ID NO: 165 is the amino acid sequence for a left CISH KO TALE-nuclease.

SEQ ID NO: 166 is the nucleotide sequence for a right CISH KO TALE-nuclease.

SEQ ID NO: 167 is the amino acid sequence for a right CISH KO TALE-nuclease.

SEQ ID NO: 168 is the nucleotide sequence for the cleavage site in the human CISH gene for CISH TALEN KO.

SEQ ID NO: 169 is the mRNA sequence for a left PD-1 KO TALE-nuclease.

SEQ ID NO: 170 is the amino acid sequence for a left PD-1 KO TALE-nuclease.

SEQ ID NO: 171 is the mRNA sequence for a right PD-1 KO TALE-nuclease.

SEQ ID NO: 172 is the amino acid sequence for a right PD-1 KO TALE-nuclease.

SEQ ID NO: 173 is the nucleotide sequence for the CISH forward primer.

SEQ ID NO: 174 is the nucleotide sequence for the CISH reverse primer.

SEQ ID NO: 175 is the nucleotide sequence for the target site in the human CISH gene for CISH TALEN KO.

### DETAILED DESCRIPTION OF THE INVENTION

### I. Introduction

Current expansion protocols give little insight into the health of the TIL that will be infused into the patient. T cells undergo a profound metabolic shift during the course of their maturation from naïve to effector T cells (see Chang, et al., Nat. Immunol. 2016, 17, 364, hereby expressly incorporated in its entirety, and in particular for the discussion and markers of anaerobic and aerobic metabolism). For example, naïve T cells rely on mitochondrial respiration to produce ATP, while mature, healthy effector T cells such as TIL are highly glycolytic, relying on aerobic glycolysis to provide the bioenergetics substrates they require for proliferation, migration, activation, and anti-tumor efficacy.

Previous papers report that limiting glycolysis and promoting mitochondrial metabolism in TILs prior to transfer is desirable as cells that are relying heavily on glycolysis will suffer nutrient deprivation upon adoptive transfer which results in a majority of the transferred cells dying. Thus, the art teaches that promoting mitochondrial metabolism might promote in vivo longevity and in fact suggests using inhibitors of glycolysis before induction of the immune response. See, Chang, et al., Nat. Immunol. 2016, 17(364).

The present invention is further directed in some embodiments to enhancing the therapeutic effect of TILs with the use of gene editing technology. While adoptive transfer of tumor infiltrating lymphocytes (TILs) offers a promising and effective therapy, there is a strong need for more effective TIL therapies that can increase a patient's response rate and response robustness. As described herein, embodiments of the present invention provide methods for expanding TILs into a therapeutic population that is gene-edited to provide an enhanced therapeutic effect.

### II. Definitions

Unless defined otherwise, all technical and scientific terms used herein have the same meaning as is commonly understood by one of skill in the art to which this invention belongs. All patents and publications referred to herein are incorporated by reference in their entireties.

The terms “co-administration,” “co-administering,” “administered in combination with,” “administering in combination with,” “simultaneous,” and “concurrent,” as used herein, encompass administration of two or more active pharmaceutical ingredients (in a preferred embodiment of the present invention, for example, a plurality of TILs) to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “ex vivo” refers to an event which involves treating or performing a procedure on a cell, tissue and/or organ which has been removed from a subject's body. Aptly, the cell, tissue and/or organ may be returned to the subject's body in a method of surgery or treatment.

As used herein “tumor infiltrating lymphocytes” or “TILs” herein is meant a population of cells originally obtained as white blood cells that have left the bloodstream of a subject and migrated into a tumor. TILs include, but are not limited to, CD8⁺ cytotoxic T cells (lymphocytes), Th1 and Th17 CD4⁺ T cells, natural killer cells, dendritic cells and M1 macrophages. TILs can generally be defined either biochemically, using cell surface markers, or functionally, by their ability to infiltrate tumors and effect treatment. TILs can be generally categorized by expressing one or more of the following biomarkers: CD4, CD8, TCR ap, CD27, CD28, CD56, CCR7, CD45Ra, CD95, PD-1, and CD25. Additionally, and alternatively, TILs can be functionally defined by their ability to infiltrate solid tumors upon reintroduction into a patient. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated as discussed herein, including, but not limited to bulk TILs and expanded TILs (“REP TILs” or “post-REP TILs”), and are genetically modified to comprise one or more Transcription activator-like effector nucleases (TALE-nuclease) able to selectively inactivate by DNA cleavage a gene encoding CISH, wherein the one or more TALE-nucleases comprise a TALE-nuclease that is directed against a target site in the gene encoding CISH, which target site comprises the nucleic acid sequence of SEQ ID NO: 175 and TIL cell populations can include these genetically modified TILs.

As used herein, “population of cells” (including TILs) herein is meant a number of cells that share common traits. In general, populations generally range from 1×10⁶ to 1×10¹⁰ in number, with different TIL populations comprising different numbers. For example, initial growth of primary TILs in the presence of IL-2 results in a population of bulk TILs of roughly 1×10⁸ cells. REP expansion is generally done to provide populations of 1.5×10⁹ to 1.5×10¹⁰ cells for infusion. At least a plurality of TILs in the population are genetically modified by one or more Transcription activator-like effector nucleases (TALE-nuclease) to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence.

By “cryopreserved TILs” herein is meant that TILs, either primary, bulk, or expanded, that include genetically modified TILs which are genetically modified by one or more Transcription activator-like effector nucleases (TALE-nuclease) to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and that are treated and stored in the range of about −150° C. to −60° C. General methods for cryopreservation are also described elsewhere herein, including in the Examples. For clarity, “cryopreserved TILs” are distinguishable from frozen tissue samples which may be used as a source of primary TILs.

By “thawed cryopreserved TILs” herein is meant a population of TILs that was previously cryopreserved and then treated to return to room temperature or higher, including but not limited to cell culture temperatures or temperatures wherein TILs may be administered to a patient.

The term “cryopreservation media” or “cryopreservation medium” refers to any medium that can be used for cryopreservation of cells. Such media can include media comprising 7% to 10% DMSO. Exemplary media include CryoStor CS10, Hyperthermasol, as well as combinations thereof. The term “CS10” refers to a cryopreservation medium which is obtained from Stemcell Technologies or from Biolife Solutions. The CS10 medium may be referred to by the trade name “CryoStor® CS10”. The CS10 medium is a serum-free, animal component-free medium which comprises DMSO.

The term “central memory T cell” refers to a subset of T cells that in the human are CD45R0+ and constitutively express CCR7 (CCR7^{hi}) and CD62L (CD62^{hi}). The surface phenotype of central memory T cells also includes TCR, CD3, CD127 (IL-7R), and IL-15R. Transcription factors for central memory T cells include BCL-6, BCL-6B, MBD2, and BMI1. Central memory T cells primarily secret IL-2 and CD40L as effector molecules after TCR triggering. Central memory T cells are predominant in the CD4 compartment in blood, and in the human are proportionally enriched in lymph nodes and tonsils.

The term “effector memory T cell” refers to a subset of human or mammalian T cells that, like central memory T cells, are CD45R0+, but have lost the constitutive expression of CCR7 (CCR7^{lo}) and are heterogeneous or low for CD62L expression (CD62L^{lo}). The surface phenotype of central memory T cells also includes TCR, CD3, CD127 (IL-7R), and IL-15R. Transcription factors for central memory T cells include BLIMP1. Effector memory T cells rapidly secret high levels of inflammatory cytokines following antigenic stimulation, including interferon-γ, IL-4, and IL-5. Effector memory T cells are predominant in the CD8 compartment in blood, and in the human are proportionally enriched in the lung, liver, and gut. CD8+ effector memory T cells carry large amounts of perform.

The terms “fragmenting,” “fragment,” and “fragmented,” as used herein to describe processes for disrupting a tumor, includes mechanical fragmentation methods such as crushing, slicing, dividing, and morcellating tumor tissue as well as any other method for disrupting the physical structure of tumor tissue.

The terms “peripheral blood mononuclear cells” and “PBMCs” refers to a peripheral blood cell having a round nucleus, including lymphocytes (T cells, B cells, NK cells) and monocytes. When used as an antigen presenting cell (PBMCs are a type of antigen-presenting cell), the peripheral blood mononuclear cells are preferably irradiated allogeneic peripheral blood mononuclear cells.

The terms “peripheral blood lymphocytes” and “PBLs” refer to T cells expanded from peripheral blood. In some embodiments, PBLs are separated from whole blood or apheresis product from a donor. In some embodiments, PBLs are separated from whole blood or apheresis product from a donor by positive or negative selection of a T cell phenotype, such as the T cell phenotype of CD3+ CD45+.

The term “anti-CD3 antibody” refers to an antibody or variant thereof, e.g., a monoclonal antibody and including human, humanized, chimeric or murine antibodies which are directed against the CD3 receptor in the T cell antigen receptor of mature T cells. Anti-CD3 antibodies include OKT-3, also known as muromonab. Anti-CD3 antibodies also include the UHCT1 clone, also known as T3 and CD3c. Other anti-CD3 antibodies include, for example, otelixizumab, teplizumab, and visilizumab.

The term “OKT-3” (also referred to herein as “OKT3”) refers to a monoclonal antibody or biosimilar or variant thereof, including human, humanized, chimeric, or murine antibodies, directed against the CD3 receptor in the T cell antigen receptor of mature T cells, and includes commercially-available forms such as OKT-3 (30 ng/mL, MACS GMP CD3 pure, Miltenyi Biotech, Inc., San Diego, CA, USA) and muromonab or variants, conservative amino acid substitutions, glycoforms, or biosimilars thereof. The amino acid sequences of the heavy and light chains of muromonab are given in Table 1 (SEQ ID NO: 1 and SEQ ID NO: 2). A hybridoma capable of producing OKT-3 is deposited with the American Type Culture Collection and assigned the ATCC accession number CRL 8001. A hybridoma capable of producing OKT-3 is also deposited with European Collection of Authenticated Cell Cultures (ECACC) and assigned Catalogue No. 86022706.

TABLE 1
Amino acid sequences of muromonab (exemplary OKT-3 antibody).

| Identifier | Sequence (One-Letter Amino Acid Symbols) | |
|---|---|---|
| SEQ ID NO: 1 | QVQLQQSGAE LARPGASVKM SCKASGYTFT RYTMHWVKQR PGQGLEWIGY INPSRGYTNY | 60 |
| Muromonab heavy | NQKFKDKATL TTDKSSSTAY MQLSSLTSED SAVYYCARYY DDHYCLDYWG QGTTLTVSSA | 120 |
| chain | KTTAPSVYPL APVCGGTTGS SVTLGCLVKG YFPEPVTLTW NSGSLSSGVH TFPAVLQSDL | 180 |
| | YTLSSSVTVT SSTWPSQSIT CNVAHPASST KVDKKIEPRP KSCDKTHTCP PCPAPELLGG | 240 |
| | PSVFLFPPKP KDTLMISRTP EVTCVVVDVS HEDPEVKFNW YVDGVEVHNA KTKPREEQYN | 300 |
| | STYRVVSVLT VLHQDWLNGK EYKCKVSNKA LPAPIEKTIS KAKGQPREPQ VYTLPPSRDE | 360 |
| | LTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 450 |
| SEQ ID NO: 2 | QIVLTQSPAI MSASPGEKVT MTCSASSSVS YMNWYQQKSG TSPKRWIYDT SKLASGVPAH | 60 |
| Muromonab light | FRGSGSGTSY SLTISGMEAE DAATYYCQQW SSNPFTFGSG TKLEINRADT APTVSIFPPS | 120 |
| chain | SEQLTSGGAS VVCFLNNFYP KDINVKWKID GSERQNGVLN SWTDQDSKDS TYSMSSTLTL | 180 |
| | TKDEYERHNS YTCEATHKTS TSPIVKSFNR NEC | 213 |

The term “IL-2” (also referred to herein as “IL2”) refers to the T cell growth factor known as interleukin-2, and includes all forms of IL-2 including human and mammalian forms, conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated by reference herein. The amino acid sequence of recombinant human IL-2 suitable for use in the invention is given in Table 2 (SEQ ID NO: 3). For example, the term IL-2 encompasses human, recombinant forms of IL-2 such as aldesleukin (PROLEUKIN, available commercially from multiple suppliers in 22 million IU per single use vials), as well as the form of recombinant IL-2 commercially supplied by CellGenix, Inc., Portsmouth, NH, USA (CELLGRO GMP) or ProSpec-Tany TechnoGene Ltd., East Brunswick, NJ, USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The amino acid sequence of aldesleukin suitable for use in the invention is given in Table 2 (SEQ ID NO: 4). The term IL-2 also encompasses pegylated forms of IL-2, as described herein, including the pegylated IL2 prodrug NKTR-214, available from Nektar Therapeutics, South San Francisco, CA, USA. NKTR-214 and pegylated IL-2 suitable for use in the invention is described in U.S. Patent Application Publication No. US 2014/0328791 A1 and International Patent Application Publication No. WO 2012/065086 A1, the disclosures of which are incorporated by reference herein. Alternative forms of conjugated IL-2 suitable for use in the invention are described in U.S. Pat. Nos. 4,766,106, 5,206,344, 5,089,261 and 4902,502, the disclosures of which are incorporated by reference herein. Formulations of IL-2 suitable for use in the invention are described in U.S. Pat. No. 6,706,289, the disclosure of which is incorporated by reference herein.

TABLE 2
Amino acid sequences of interleukins.

| Identifier | Sequence (One-Letter Amino Acid Symbols) | |
|---|---|---|
| SEQ ID NO: 3 | MAPTSSSTKK TQLQLEHLLL DLQMILNGIN NYKNPKLTRM LTFKFYMPKK ATELKHLQCL | 60 |
| recombinant | EEELKPLEEV LNLAQSKNFH LRPRDLISNI NVIVLELKGS ETTFMCEYAD ETATIVEFLN | 120 |
| human IL-2 | RWITFCQSII STLT | 134 |
| (rhIL-2) | | |
| SEQ ID NO: 4 | PTSSSTKKTQ LQLEHLLLDL QMILNGINNY KNPKLTRMLT FKFYMPKKAT ELKHLQCLEE | 60 |
| Aldesleukin | ELKPLEEVLN LAQSKNFHLR PRDLISNINV IVLELKGSET TFMCEYADET ATIVEFLNRW | 120 |
| | ITFSQSIIST LT | 132 |
| SEQ ID NO: 5 | MHKCDITLQE IIKTINSLTE QKTLCTELTV TDIFAASKNT TEKETFCRAA TVLRQFYSHH | 60 |
| recombinant | EKDTRCLGAT AQQFHRHKQL IRFLKRLDRN LWGLAGLNSC PVKEANQSTL ENFLERLKTI | 120 |
| human IL-4 | MREKYSKCSS | 130 |
| (rhIL-4) | | |
| SEQ ID NO: 6 | MDCDIEGKDG KQYESVLMVS IDQLLDSMKE IGSNCLNNEF NFFKRHICDA NKEGMFLFRA | 60 |
| recombinant | ARKLRQFLKM NSTGDFDLHL LKVSEGTTIL LNCTGQVKGR KPAALGEAQP TKSLEENKSL | 120 |
| human IL-7 | KEQKKLNDLC FLKRLLQEIK TCWNKILMGT KEH | 153 |
| (rhIL-7) | | |
| SEQ ID NO: 7 | MNWVNVISDL KKIEDLIQSM HIDATLYTES DVHPSCKVTA MKCFLLELQV ISLESGDASI | 60 |
| recombinant | HDTVENLIIL ANNSLSSNGN VTESGCKECE ELEEKNIKEF LQSFVHIVQM FINTS | 115 |
| human IL-15 | | |
| (rhIL-15) | | |
| SEQ ID NO: 8 | MQDRHMIRMR QLIDIVDQLK NYVNDLVPEF LPAPEDVETN CEWSAFSCFQ KAQLKSANTG | 60 |
| recombinant | NNERIINVSI KKLKRKPPST NAGRRQKHRL TCPSCDSYEK KPPKEFLERF KSLLQKMIHQ | 120 |
| human IL-21 | HLSSRTHGSE DS | 132 |
| (rhIL-21) | | |

The term “IL-4” (also referred to herein as “IL4”) refers to the cytokine known as interleukin 4, which is produced by Th2 T cells and by eosinophils, basophils, and mast cells. IL-4 regulates the differentiation of naïve helper T cells (Th0 cells) to Th2 T cells. Steinke and Borish, Respir. Res. 2001, 2, 66-70. Upon activation by IL-4, Th2 T cells subsequently produce additional IL-4 in a positive feedback loop. IL-4 also stimulates B cell proliferation and class II MHC expression, and induces class switching to IgE and IgG₁ expression from B cells. Recombinant human IL-4 suitable for use in the invention is commercially available from multiple suppliers, including ProSpec-Tany TechnoGene Ltd., East Brunswick, NJ, USA (Cat. No. CYT-211) and ThermoFisher Scientific, Inc., Waltham, MA, USA (human IL-15 recombinant protein, Cat. No. Gibco CTP0043). The amino acid sequence of recombinant human IL-4 suitable for use in the invention is given in Table 2 (SEQ ID NO: 5).

The term “IL-7” (also referred to herein as “IL7”) refers to a glycosylated tissue-derived cytokine known as interleukin 7, which may be obtained from stromal and epithelial cells, as well as from dendritic cells. Fry and Mackall, Blood 2002, 99, 3892-904. IL-7 can stimulate the development of T cells. IL-7 binds to the IL-7 receptor, a heterodimer consisting of IL-7 receptor alpha and common gamma chain receptor, which in a series of signals important for T cell development within the thymus and survival within the periphery. Recombinant human IL-7 suitable for use in the invention is commercially available from multiple suppliers, including ProSpec-Tany TechnoGene Ltd., East Brunswick, NJ, USA (Cat. No. CYT-254) and ThermoFisher Scientific, Inc., Waltham, MA, USA (human IL-15 recombinant protein, Cat. No. Gibco PHC0071). The amino acid sequence of recombinant human IL-7 suitable for use in the invention is given in Table 2 (SEQ ID NO: 6).

The term “IL-15” (also referred to herein as “IL15”) refers to the T cell growth factor known as interleukin-15, and includes all forms of IL-2 including human and mammalian forms, conservative amino acid substitutions, glycoforms, biosimilars, and variants thereof. IL-15 is described, e.g., in Fehniger and Caligiuri, Blood 2001, 97, 14-32, the disclosure of which is incorporated by reference herein. IL-15 shares β and γ signaling receptor subunits with IL-2. Recombinant human IL-15 is a single, non-glycosylated polypeptide chain containing 114 amino acids (and an N-terminal methionine) with a molecular mass of 12.8 kDa. Recombinant human IL-15 is commercially available from multiple suppliers, including ProSpec-Tany TechnoGene Ltd., East Brunswick, NJ, USA (Cat. No. CYT-230-b) and ThermoFisher Scientific, Inc., Waltham, MA, USA (human IL-15 recombinant protein, Cat. No. 34-8159-82). The amino acid sequence of recombinant human IL-15 suitable for use in the invention is given in Table 2 (SEQ ID NO: 7).

The term “IL-21” (also referred to herein as “IL21”) refers to the pleiotropic cytokine protein known as interleukin-21, and includes all forms of IL-21 including human and mammalian forms, conservative amino acid substitutions, glycoforms, biosimilars, and variants thereof. IL-21 is described, e.g., in Spolski and Leonard, Nat. Rev. Drug. Disc. 2014, 13, 379-95, the disclosure of which is incorporated by reference herein. IL-21 is primarily produced by natural killer T cells and activated human CD4⁺ T cells. Recombinant human IL-21 is a single, non-glycosylated polypeptide chain containing 132 amino acids with a molecular mass of 15.4 kDa. Recombinant human IL-21 is commercially available from multiple suppliers, including ProSpec-Tany TechnoGene Ltd., East Brunswick, NJ, USA (Cat. No. CYT-408-b) and ThermoFisher Scientific, Inc., Waltham, MA, USA (human IL-21 recombinant protein, Cat. No. 14-8219-80). The amino acid sequence of recombinant human IL-21 suitable for use in the invention is given in Table 2 (SEQ ID NO: 8).

When “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the tumor infiltrating lymphocytes (e.g. secondary TILs or genetically modified cytotoxic lymphocytes) described herein may be administered at a dosage of 10⁴ to 10¹¹ cells/kg body weight (e.g., 10⁵ to 10⁶, 10⁵ to 10¹⁰, 10⁵ to 10¹¹, 10⁶ to 10¹⁰, 10⁶ to 10¹¹, 10⁷ to 10¹¹, 10⁷ to 10¹⁰, 10⁸ to 10¹¹, 10⁸ to 10¹⁰, 10⁹ to 10¹¹, or 10⁹ to 10¹⁰ cells/kg body weight), including all integer values within those ranges. Tumor infiltrating lymphocytes (including in all cases, at least a plurality of cytotoxic lymphocytes genetically modified by introducing into the cytotoxic lymphocytes nucleic acids, such as mRNAs, encoding one or more Transcription activator-like effector nucleases (TALE-nuclease) to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence) compositions may also be administered multiple times at these dosages. The tumor infiltrating lymphocytes (including in all cases, at least a plurality of cytotoxic lymphocytes genetically modified by introducing into the cytotoxic lymphocytes nucleic acids, such as mRNAs, encoding one or more Transcription activator-like effector nucleases (TALE-nuclease) to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence) can be administered by using infusion techniques that are commonly known in immunotherapy (see, e.g., Rosenberg et al., New Eng. J of Med., 319: 1676, 1988). The optimal dosage and treatment regime for a particular patient can readily be determined by one skilled in the art of medicine by monitoring the patient for signs of disease and adjusting the treatment accordingly.

The term “microenvironment,” as used herein, may refer to the solid or hematological tumor microenvironment as a whole or to an individual subset of cells within the microenvironment. The tumor microenvironment, as used herein, refers to a complex mixture of “cells, soluble factors, signaling molecules, extracellular matrices, and mechanical cues that promote neoplastic transformation, support tumor growth and invasion, protect the tumor from host immunity, foster therapeutic resistance, and provide niches for dominant metastases to thrive,” as described in Swartz, et al., Cancer Res., 2012, 72, 2473. Although tumors express antigens that should be recognized by T cells, tumor clearance by the immune system is rare because of immune suppression by the microenvironment.

In some embodiments, the invention includes a method of treating a cancer with a population of TILs (at least a plurality of TILs in which population are genetically modified by introducing into the TILs nucleic acids, such as mRNAs, encoding one or more Transcription activator-like effector nucleases (TALE-nuclease) to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence), wherein a patient is pre-treated with non-myeloablative chemotherapy prior to an infusion of such TILs according to the invention. In some embodiments, the population of TILs may be provided wherein a patient is pre-treated with nonmyeloablative chemotherapy prior to an infusion of such TILs according to the present invention. In some embodiments, the non-myeloablative chemotherapy is cyclophosphamide 60 mg/kg/d for 2 days (days 27 and 26 prior to infusion of such TILs) and fludarabine 25 mg/m²/d for 5 days (days 27 to 23 prior to infusion of such TILs). In some embodiments, after non-myeloablative chemotherapy and TIL infusion (at day 0) according to the invention, the patient receives an intravenous infusion of IL-2 intravenously at 720,000 IU/kg every 8 hours to physiologic tolerance.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, or coding regions from different sources. Similarly, a heterologous protein indicates that the protein comprises two or more subsequences that are not found in the same relationship to each other in nature (e.g., a fusion protein).

The terms “sequence identity,” “percent identity,” and “sequence percent identity” (or synonyms thereof, e.g., “99% identical”) in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, California) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

As used herein, the term “variant” encompasses but is not limited to antibodies or fusion proteins which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody. The term variant also includes pegylated antibodies or proteins. TILs can generally be defined either biochemically, using cell surface markers, or functionally, by their ability to infiltrate tumors and effect treatment. TILs can be generally categorized by expressing one or more of the following biomarkers: CD4, CD8, TCR αβ, CD27, CD28, CD56, CCR7, CD45Ra, CD95, PD-1, and CD25. Additionally, and alternatively, TILs can be functionally defined by their ability to infiltrate solid tumors upon reintroduction into a patient. TILS may further be characterized by potency—for example, TILS may be considered potent if, for example, interferon (IFN) release is greater than about 50 pg/mL, greater than about 100 pg/mL, greater than about 150 pg/mL, or greater than about 200 pg/mL.

The term “deoxyribonucleotide” encompasses natural and synthetic, unmodified and modified deoxyribonucleotides. Modifications include changes to the sugar moiety, to the base moiety and/or to the linkages between deoxyribonucleotide in the oligonucleotide.

The term “RNA” defines a molecule comprising at least one ribonucleotide residue. The term “ribonucleotide” defines a nucleotide with a hydroxyl group at the 2′ position of a b-D-ribofuranose moiety. The term RNA includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Nucleotides of the RNA molecules described herein may also comprise non-standard nucleotides, such as non-naturally occurring nucleotides or chemically synthesized nucleotides or deoxynucleotides. These altered RNAs can be referred to as analogs or analogs of naturally-occurring RNA.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in therapeutic compositions of the invention is contemplated. Additional active pharmaceutical ingredients, such as other drugs, can also be incorporated into the described compositions and methods.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of.”

The terms “antibody” and its plural form “antibodies” refer to whole immunoglobulins and any antigen-binding fragment (“antigen-binding portion”) or single chains thereof. An “antibody” further refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions of an antibody may be further subdivided into regions of hypervariability, which are referred to as complementarity determining regions (CDR) or hypervariable regions (HVR), and which can be interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen epitope or epitopes. The constant regions of the antibodies may mediate the binding of the immunoglobulin to host tissues or factors, including various cells of the immune system (e.g., effector cells) and the first component (Clq) of the classical complement system.

The term “antigen” refers to a substance that induces an immune response. In some embodiments, an antigen is a molecule capable of being bound by an antibody or a TCR if presented by major histocompatibility complex (MHC) molecules. The term “antigen”, as used herein, also encompasses T cell epitopes. An antigen is additionally capable of being recognized by the immune system. In some embodiments, an antigen is capable of inducing a humoral immune response or a cellular immune response leading to the activation of B lymphocytes and/or T lymphocytes. In some cases, this may require that the antigen contains or is linked to a Th cell epitope. An antigen can also have one or more epitopes (e.g., B- and T-epitopes). In some embodiments, an antigen will preferably react, typically in a highly specific and selective manner, with its corresponding antibody or TCR and not with the multitude of other antibodies or TCRs which may be induced by other antigens.

The terms “monoclonal antibody,” “mAb,” “monoclonal antibody composition,” or their plural forms refer to a preparation of antibody molecules of single molecular composition. A monoclonal antibody composition displays a single binding specificity and affinity for a particular epitope. Monoclonal antibodies specific to certain receptors can be made using knowledge and skill in the art of injecting test subjects with suitable antigen and then isolating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

The terms “antigen-binding portion” or “antigen-binding fragment” of an antibody (or simply “antibody portion” or “fragment”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a domain antibody (dAb) fragment (Ward, et al., Nature, 1989, 341, 544-546), which may consist of a V_{H} or a V_{L} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); See, e.g., Bird, et al., Science 1988, 242, 423-426; and Huston, et al., Proc. Natl. Acad. Sci. USA 1988, 85, 5879-5883). Such scFv antibodies are also intended to be encompassed within the terms “antigen-binding portion” or “antigen-binding fragment” of an antibody. These antibody fragments are obtained using conventional techniques known to those with skill in the art, and the fragments are screened for utility in the same manner as are intact antibodies.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). The term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In some embodiments, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (such as a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, including a conjugate of the antibody and another active pharmaceutical ingredient or antibody. The terms “conjugate,” “antibody-drug conjugate”, “ADC,” or “immunoconjugate” refers to an antibody, or a fragment thereof, conjugated to another therapeutic moiety, which can be conjugated to antibodies described herein using methods available in the art.

The terms “humanized antibody,” “humanized antibodies,” and “humanized” are intended to refer to antibodies in which CDR sequences derived from the germline of another mammalian species, such as a mouse, have been grafted onto human framework sequences. Additional framework region modifications may be made within the human framework sequences. Humanized forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a 15 hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fe), typically that of a human immunoglobulin. For further details, see Jones, et al., Nature 1986, 321, 522-525; Riechmann, et al., Nature 1988, 332, 323-329; and Presta, Curr. Op. Struct. Biol. 1992, 2, 593-596. The antibodies described herein may also be modified to employ any Fc variant which is known to impart an improvement (e.g., reduction) in effector function and/or FcR binding. The Fc variants may include, for example, any one of the amino acid substitutions disclosed in International Patent Application Publication Nos. WO 1988/07089 A1, WO 1996/14339 A1, WO 1998/05787 A1, WO 1998/23289 A1, WO 1999/51642 A1, WO 99/58572 A1, WO 2000/09560 A2, WO 2000/32767 A1, WO 2000/42072 A2, WO 2002/44215 A2, WO 2002/060919 A2, WO 2003/074569 A2, WO 2004/016750 A2, WO 2004/029207 A2, WO 2004/035752 A2, WO 2004/063351 A2, WO 2004/074455 A2, WO 2004/099249 A2, WO 2005/040217 A2, WO 2005/070963 A1, WO 2005/077981 A2, WO 2005/092925 A2, WO 2005/123780 A2, WO 2006/019447 A1, WO 2006/047350 A2, and WO 2006/085967 A2; and U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784; the disclosures of which are incorporated by reference herein.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

A “diabody” is a small antibody fragment with two antigen-binding sites. The fragments comprises a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., European Patent No. EP 404,097, International Patent Publication No. WO 93/11161; and Bolliger, et al., PNAS, 1993, 90, 6444-6448.

The term “glycosylation” refers to a modified derivative of an antibody. An aglycoslated antibody lacks glycosylation. Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Altered glycosylation may increase the affinity of the antibody for antigen, as described in U.S. Pat. Nos. 5,714,350 and 6,350,861. Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see e.g. U.S. Patent Publication No. 2004/0110704 or Yamane-Ohnuki, et al., Biotechnol. Bioeng., 2004, 87, 614-622). As another example, European Patent No. EP 1,176,195 describes a cell line with a functionally disrupted FUT8 gene, which encodes a fucosyl transferase, such that antibodies expressed in such a cell line exhibit hypofucosylation by reducing or eliminating the alpha 1,6 bond-related enzyme, and also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). International Patent Publication WO 03/035835 describes a variant CHO cell line, Lec 13 cells, with reduced ability to attach fucose to Asn(297)-linked carbohydrates, also resulting in hypofucosylation of antibodies expressed in that host cell (see also Shields, et al., J. Biol. Chem. 2002, 277, 26733-26740. International Patent Publication WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana, et al., Nat. Biotech. 1999, 17, 176-180). Alternatively, the fucose residues of the antibody may be cleaved off using a fucosidase enzyme. For example, the fucosidase alpha-L-fucosidase removes fucosyl residues from antibodies as described in Tarentino, et al., Biochem. 1975, 14, 5516-5523.

“Pegylation” refers to a modified antibody, or a fragment thereof, that typically is reacted with polyethylene glycol (PEG), such as a reactive ester or aldehyde derivative of PEG, under conditions in which one or more PEG groups become attached to the antibody or antibody fragment. Pegylation may, for example, increase the biological (e.g., serum) half-life of the antibody. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀)alkoxy- or aryloxy-polyethylene glycol or polyethylene glycol-maleimide. The antibody to be pegylated may be an aglycosylated antibody. Methods for pegylation are known in the art and can be applied to the antibodies of the invention, as described for example in European Patent Nos. EP 0154316 and EP 0401384 and U.S. Pat. No. 5,824,778, the disclosures of each of which are incorporated by reference herein.

The term “biosimilar” means a biological product, including a monoclonal antibody or protein, that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. They can consist of relatively small molecules such as human insulin or erythropoietin, or complex molecules such as monoclonal antibodies. For example, if the reference IL-2 protein is aldesleukin (PROLEUKIN), a protein approved by drug regulatory authorities with reference to aldesleukin is a “biosimilar to” aldesleukin or is a “biosimilar thereof” of aldesleukin. In Europe, a similar biological or “biosimilar” medicine is a biological medicine that is similar to another biological medicine that has already been authorized for use by the European Medicines Agency (EMA). The relevant legal basis for similar biological applications in Europe is Article 6 of Regulation (EC) No 726/2004 and Article 10(4) of Directive 2001/83/EC, as amended and therefore in Europe, the biosimilar may be authorized, approved for authorization or subject of an application for authorization under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA and published on its website. A biosimilar as described herein may be similar to the reference medicinal product by way of quality characteristics, biological activity, mechanism of action, safety profiles and/or efficacy. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorized by the EMA. However, in some instances, the biosimilar may be compared to a biological medicinal product which has been authorized outside the European Economic Area (a non-EEA authorized “comparator”) in certain studies. Such studies include for example certain clinical and in vivo non-clinical studies. As used herein, the term “biosimilar” also relates to a biological medicinal product which has been or may be compared to a non-EEA authorized comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97% or greater to the amino acid sequence of its reference medicinal product, e.g., 97%, 98%, 99% or 100%. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorized or considered suitable for authorization. In certain circumstances, the biosimilar exhibits different binding characteristics as compared to the reference medicinal product, wherein the different binding characteristics are considered by a Regulatory Authority such as the EMA not to be a barrier for authorization as a similar biological product. The term “biosimilar” is also used synonymously by other national and regional regulatory agencies.

### III. TALEN Gene-Editing and Expansion Processes

a. Overview: TIL Expansion+TALEN Gene-Editing

Embodiments of the present invention are directed to methods for expanding TIL populations, the methods comprising one or more steps of TALEN gene-editing at least a portion of the TILs by introducing into the TILs nucleic acids, such as mRNAs, encoding one or more transcription activator-like effector nucleases (TALE-nuclease) to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, in order to enhance their therapeutic effect. As used herein, “TALEN gene-editing,” “gene editing,” and “genome editing” refer to a type of genetic modification in which DNA is permanently modified in the genome of a cell, e.g., DNA is inserted, deleted, modified or replaced within the cell's genome. In some embodiments, TALEN gene-editing causes the expression of a DNA sequence to be silenced (sometimes referred to as a gene knockout) or inhibited/reduced (sometimes referred to as a gene knockdown). In other embodiments, TALEN gene-editing causes the expression of a DNA sequence to be enhanced (e.g., by causing over-expression). In accordance with embodiments of the present invention, TALEN gene-editing technology is used to enhance the effectiveness of a therapeutic population of TILs.

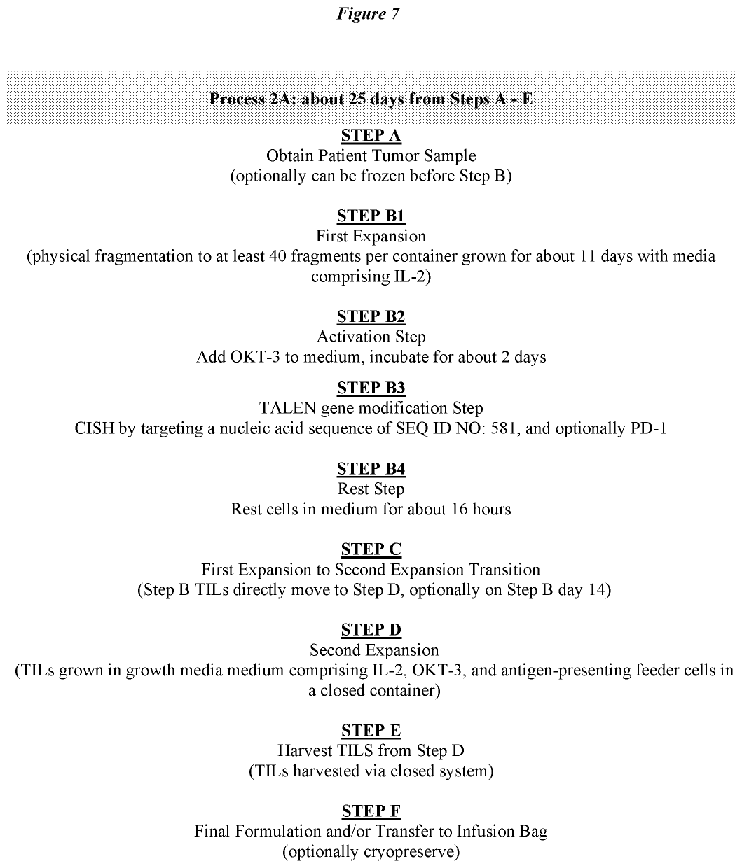

The genetically modified TILs of the invention comprise a population of TILS at least a portion of which are genetically modified by introducing into the TLS nucleic acids, such as mRNAs, encoding one or more TALE-nucleases directed to selectively inactivate by DNA cleavage a gene encoding CISH, wherein the one or more TALE-nucleases comprise a TALE-nuclease that is directed against a nucleic acid sequence comprising the nucleic acid sequence of SEQ ID NO: 175, which population of TILs can be expanding into a therapeutic population in accordance with any embodiment of the methods as described in FIG. 7 herein or as described in PCT/US2017/058610, PCT/US2018/012605, or PCT/US2018/012633.

### B. Timing of TALEN Gene-Editing During TIL Expansion

According to some embodiments, the invention provides a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2, and optionally OKT-3 (e.g., OKT-3 may be present in the culture medium beginning on the start date of the expansion process), to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (c) to step (d) occurs without opening the system;
(e) harvesting the therapeutic population of TILs obtained from step (d), wherein the transition from step (d) to step (e) occurs without opening the system;
(f) transferring the harvested TIL population from step (e) to an infusion bag, wherein the transfer from step (e) to (f) occurs without opening the system; and
(g) at any time during the method prior to the transfer to the infusion bag in step (f), subjecting at least a portion of the TIL cells to gene editing by introducing into the TIL cells nucleic acids, optionally mRNAs, encoding one or more TALE-nucleases directed to selectively inactivate by DNA cleavage a gene encoding CISH, wherein the one or more TALE-nucleases comprise a TALE-nuclease that is directed against a nucleic acid sequence comprising the nucleic acid sequence of SEQ ID NO: 175.

As stated in step (g) of the embodiments described above, the gene-editing process may be carried out at any time during the TIL expansion method prior to the transfer to the infusion bag in step (f), which means that the gene editing may be carried out on TILs before, during, or after any of the steps in the expansion method; for example, during any of steps (a)-(f) outlined in the method above, or before or after any of steps (a)-(e) outlined in the method above. According to certain embodiments, TILs are collected during the expansion method (e.g., the expansion method is “paused” for at least a portion of the TILs), and the collected TILs are subjected to the gene-editing process, and, in some cases, subsequently reintroduced back into the expansion method (e.g., back into the culture medium) to continue the expansion process, so that at least a portion of the therapeutic population of TILs that are eventually transferred to the infusion bag are permanently gene-edited. In some embodiments, the gene-editing process may be carried out before expansion by activating TILs, performing a gene-editing step on the activated TILs, and expanding the gene-edited TILs according to the processes described herein.

It should be noted that alternative embodiments of the expansion process may differ from the method shown above; e.g., alternative embodiments may not have the same steps (a)-(g), or may have a different number of steps. Regardless of the specific embodiment, the gene-editing process may be carried out at any time during the TIL expansion method. For example, alternative embodiments may include more than two expansions, and it is possible that gene-editing may be conducted on the TILs during a third or fourth expansion, etc.

According to one embodiment, the gene-editing process is carried out on TILs from one or more of the first population, the second population, and the third population. For example, gene-editing may be carried out on the first population of TILs, or on a portion of TILs collected from the first population, and following the gene-editing process those TILs may subsequently be placed back into the expansion process (e.g., back into the culture medium). Alternatively, gene-editing may be carried out on TILs from the second or third population, or on a portion of TILs collected from the second or third population, respectively, and following the gene-editing process those TILs may subsequently be placed back into the expansion process (e.g., back into the culture medium). According to other embodiments, gene-editing is performed while the TILs are still in the culture medium and while the expansion is being carried out, i.e., they are not necessarily “removed” from the expansion in order to conduct gene-editing.

According to other embodiments, the gene-editing process is carried out on TILs from the first expansion, or TILs from the second expansion, or both. For example, during the first expansion or second expansion, gene-editing may be carried out on TILs that are collected from the culture medium, and following the gene-editing process those TILs may subsequently be placed back into the expansion method, e.g., by reintroducing them back into the culture medium.

According to other embodiments, the gene-editing process is carried out on at least a portion of the TILs after the first expansion and before the second expansion. For example, after the first expansion, gene-editing may be carried out on TILs that are collected from the culture medium, and following the gene-editing process those TILs may subsequently be placed back into the expansion method, e.g., by reintroducing them back into the culture medium for the second expansion.

According to alternative embodiments, the gene-editing process is carried out before step (c) (e.g., before, during, or after any of steps (a)-(b)), before step (d) (e.g., before, during, or after any of steps (a)-(c)), before step (e) (e.g., before, during, or after any of steps (a)-(d)), or before step (f) (e.g., before, during, or after any of steps (a)-(e)).

It should be noted with regard to OKT-3, according to certain embodiments, that the cell culture medium may comprise OKT-3 beginning on the start day (Day 0), or on Day 1 of the first expansion, such that the gene-editing is carried out on TILs after they have been exposed to OKT-3 in the cell culture medium on Day 0 and/or Day 1. According to other embodiments, the cell culture medium comprises OKT-3 during the first expansion and/or during the second expansion, and the gene-editing is carried out before the OKT-3 is introduced into the cell culture medium. Alternatively, the cell culture medium may comprise OKT-3 during the first expansion and/or during the second expansion, and the gene-editing is carried out after the OKT-3 is introduced into the cell culture medium.

It should also be noted with regard to a 4-1BB agonist, according to certain embodiments, that the cell culture medium may comprise a 4-1BB agonist beginning on the start day (Day 0), or on Day 1 of the first expansion, such that the gene-editing is carried out on TILs after they have been exposed to a 4-1BB agonist in the cell culture medium on Day 0 and/or Day 1. According to other embodiments, the cell culture medium comprises a 4-1BB agonist during the first expansion and/or during the second expansion, and the gene-editing is carried out before the 4-1BB agonist is introduced into the cell culture medium. Alternatively, the cell culture medium may comprise a 4-1BB agonist during the first expansion and/or during the second expansion, and the gene-editing is carried out after the 4-1BB agonist is introduced into the cell culture medium.

It should also be noted with regard to IL-2, according to certain embodiments, that the cell culture medium may comprise IL-2 beginning on the start day (Day 0), or on Day 1 of the first expansion, such that the gene-editing is carried out on TILs after they have been exposed to IL-2 in the cell culture medium on Day 0 and/or Day 1. According to other embodiments, the cell culture medium comprises IL-2 during the first expansion and/or during the second expansion, and the gene-editing is carried out before the IL-2 is introduced into the cell culture medium. Alternatively, the cell culture medium may comprise IL-2 during the first expansion and/or during the second expansion, and the gene-editing is carried out after the IL-2 is introduced into the cell culture medium.

As discussed above, one or more of OKT-3, 4-1BB agonist and IL-2 may be included in the cell culture medium beginning on Day 0 or Day 1 of the first expansion. According to one embodiment, OKT-3 is included in the cell culture medium beginning on Day 0 or Day 1 of the first expansion, and/or a 4-1BB agonist is included in the cell culture medium beginning on Day 0 or Day 1 of the first expansion, and/or IL-2 is included in the cell culture medium beginning on Day 0 or Day 1 of the first expansion. According to an example, the cell culture medium comprises OKT-3 and a 4-1BB agonist beginning on Day 0 or Day 1 of the first expansion. According to another example, the cell culture medium comprises OKT-3, a 4-1BB agonist and IL-2 beginning on Day 0 or Day 1 of the first expansion. Of course, one or more of OKT-3, 4-1BB agonist and IL-2 may be added to the cell culture medium at one or more additional time points during the expansion process, as set forth in various embodiments described herein.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, wherein the transition from step (c) to step (d) occurs without opening the system;
(e) sterile electroporating the second population of TILs to effect transfer into a portion of cells of the second population of TILs of one or more nucleic acids, optionally mRNAs, encoding one or more TALE-nucleases directed to selectively inactivate by DNA cleavage a gene encoding CISH, wherein the one or more TALE-nucleases comprise a TALE-nuclease that is directed against a nucleic acid sequence comprising the nucleic acid sequence of SEQ ID NO: 175;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain a third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs; and
(i) transferring the harvested TIL population to an infusion bag, wherein the transfer from step (h) to (i) occurs without opening the system,
   wherein the sterile electroporation of the one or more nucleic acids into the portion of cells of the second population of TILs modifies a plurality of cells to reduce expression of CISH in the cells.

In some embodiments, the nucleic acids are DNAs. In some embodiments, the nucleic acids are RNAs. In some embodiments, the nucleic acids are mRNAs.

According to some embodiments, the foregoing method further comprises cryopreserving the harvested TIL population using a cryopreservation medium. In some embodiments, the cryopreservation medium is a dimethylsulfoxide-based cryopreservation medium. In other embodiments, the cryopreservation medium is CS10.

### 1. CISH

CISH, a member of the suppressor of cytokine signaling (SOCS) family, is induced by TCR stimulation in CD8+ T cells and inhibits their functional avidity against tumors. Genetic deletion of CISH in CD8+ T cells may enhance their expansion, functional avidity, and cytokine polyfunctionality, resulting in pronounced and durable regression of established tumors. See, e.g., Palmer et al., Journal of Experimental Medicine, 212 (12): 2095 (2015).

According to particular embodiments, expression of CISH in TILs is silenced or reduced in accordance with compositions and methods of the present invention with the methods described as Gen 2 or Gen 3 as shown in FIG. 7, and wherein the genetically modified TILs are produced by introducing into the TILs nucleic acids, optionally mRNAs, encoding one or more TALE-nucleases able to selectively inactivate by DNA cleavage a gene encoding CISH, wherein the one or more TALE-nucleases comprise a TALE-nuclease that is directed against a nucleic acid sequence comprising the nucleic acid sequence of SEQ ID NO: 175, wherein the method comprises TALEN gene-editing at least a portion of the TILs by silencing or repressing the expression of CISH.

### 2. PD-1

One of the most studied targets for the induction of checkpoint blockade is the programmed death receptor (PD1 or PD-1, also known as PDCD1), a member of the CD28 super family of T-cell regulators. Its ligands, PD-L1 and PD-L2, are expressed on a variety of tumor cells, including melanoma. The interaction of PD-1 with PD-L1 inhibits T-cell effector function, results in T-cell exhaustion in the setting of chronic stimulation and induces T-cell apoptosis in the tumor microenvironment. PD1 may also play a role in tumor-specific escape from immune surveillance.

According to particular embodiments, the invention provides a method for expanding the genetically modified tumor infiltrating lymphocytes (TILs) into a therapeutic population of TILs, which expansion may be carried out in accordance with the methods described as Gen 2 as shown in FIG. 7, where the genetically modified TILs are produced by introducing into the TILs nucleic acids, optionally mRNAs, encoding one or more TALE-nucleases able to selectively inactivate by DNA cleavage a gene encoding CISH, wherein the one or more TALE-nucleases comprise a TALE-nuclease that is directed against one of the gene target sequences of CISH comprising the nucleic acid sequence of SEQ ID NO: 175, and wherein the method optionally further comprises TALEN gene-editing at least a portion of the TILs by silencing or repressing the expression of PD1. For example, this TALE method can be used to silence or reduce the expression of PD1 in the TILs, in addition to CISH. In some embodiments, the TALENs targeting the PD-1 gene are those described in WO 2013/176915 A1, WO 2014/184744 A1, WO 2014/184741 A1, WO 2018/007263 A1, and WO 2018/073391 A1 including any of the PD-1 TALENs described in Table 10 on pages 62-63 of WO 2013/176915 A1, any of the PD-1 TALENs described in Table 11 on page 78 of WO 2014/184744 A1, any of the PD-1 TALENs described in Table 11 on page 75 of WO 2014/184741 A1, any of the PD-1 TALENs described in Table 3 on pages 48-52 of WO 2018/007263 A1, and any of the PD-1 TALENs described in Table 4 on pages 62-68 and/or in Table 5 on pages 73-99 of WO 2018/073391 A1.

### C. TALE Gene Editing Methods

Major classes of nucleases that have been developed to enable site-specific genomic editing include transcription activator-like nucleases (TALENs), which achieve specific DNA binding via protein-DNA interactions. See, e.g., Cox et al., Nature Medicine, 2015, Vol. 21, No. 2. TALE methods, embodiments of which are described in more detail below, can be used as the gene editing method of the present invention.

As discussed above, embodiments of the present invention provide tumor infiltrating lymphocytes (TILs) that have been genetically modified via TALEN gene-editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1, to enhance their therapeutic effect. Embodiments of the present invention embrace methods of expansion of such genetically edited TILs into a population of TILs. Embodiments of the present invention also provide methods for expanding such genetically edited TILs into a therapeutic population.

In some embodiments, the invention provides a method of genetically modifying a population of TILs by electroporation of TILs with nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence. Electroporation methods are known in the art and are described, e.g., in Tsong, Biophys. J 1991, 60, 297-306, and U.S. Patent Application Publication No. 2014/0227237 A1, the disclosures of each of which are incorporated by reference herein. Other electroporation methods known in the art, such as those described in U.S. Pat. Nos. 5,019,034; 5,128,257; 5,137,817; 5,173,158; 5,232,856; 5,273,525; 5,304,120; 5,318,514; 6,010,613 and 6,078,490, the disclosures of which are incorporated by reference herein, may be used. In some embodiments, the electroporation method is a sterile electroporation method. In some embodiments, the electroporation method is a pulsed electroporation method. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse amplitude. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse width. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein a first pulse interval for a first set of two of the at least three pulses is different from a second pulse interval for a second set of two of the at least three pulses. In some embodiments, the electroporation method is a pulsed electroporation method comprising the steps of treating TILs with pulsed electrical fields to induce pore formation in the TILs, comprising the step of applying a sequence of at least three DC electrical pulses, having field strengths equal to or greater than 100 V/cm, to TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses, such that induced pores are sustained for a relatively long period of time, and such that viability of the TILs is maintained. In some embodiments, a method of genetically modifying a population of TILs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In some embodiments, a method of genetically modifying a population of TILs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In some embodiments, a method of genetically modifying a population of TILs includes the step of transfection using methods described in U.S. Pat. Nos. 5,766,902; 6,025,337; 6,410,517; 6,475,994; and 7,189,705; the disclosures of each of which are incorporated by reference herein.

In some embodiments of the present invention, electroporation is used for delivery of the desired TALEN-encoding nucleic acids, including TALEN-encoding RNAs and/or DNAs. In some embodiments of the present invention, the electroporation system is a flow electroporation system. An example of a suitable flow electroporation system suitable for use with some embodiments of the present invention is the commercially-available MaxCyte STX system. There are several alternative commercially-available electroporation instruments which may be suitable for use with the present invention, such as the AgilePulse system or ECM 830 available from BTX-Harvard Apparatus, Cellaxess Elektra (Cellectricon), Nucleofector (Lonza/Amaxa), GenePulser MXcell (BIORAD), iPorator-96 (Primax) or siPORTer96 (Ambion). In some embodiments of the present invention, the electroporation system forms a closed, sterile system with the remainder of the TIL expansion method. In some embodiments of the present invention, the electroporation system is a pulsed electroporation system as described herein, and forms a closed, sterile system with the remainder of the TIL expansion method.

Any suitable method may be used for expanding TILs that have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some methods of the invention, the expansion of such genetically edited TILs into a therapeutic population may be carried out in accordance with any embodiment of the methods as described in FIG. 7 herein or as described in PCT/US2017/058610, PCT/US2018/012605, or PCT/US2018/012633.

TALE stands for “Transcription Activator-Like Effector” proteins, which include TALENs (“Transcription Activator-Like Effector Nucleases”). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease (TALEN). TALE-nucleases are very specific reagents because they need to bind DNA by pairs under obligatory heterodimeric form to obtain dimerization of the cleavage domain Fok-1. Left and right heterodimer members each recognizes a different nucleic sequences of about 14 to 20 bp, together spanning target sequences of 30 to 50 bp overall specificity. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Strategies that enable the rapid assembly of custom TALE arrays include Golden Gate molecular cloning, high-throughput solid-phase assembly, and ligation-independent cloning techniques. Custom-designed TALE arrays are also commercially available through Cellectis Bioresearch (Paris, France), Transposagen Biopharmaceuticals (Lexington, KY, USA), and Life Technologies (Grand Island, NY, USA). Additionally web-based tools, such as TAL Effector-Nucleotide Target 2.0, are available that enable the design of custom TAL effector repeat arrays for desired targets and also provides predicted TAL effector binding sites. See Doyle, et al., Nucleic Acids Research, 2012, Vol. 40, W117-W122. Examples of TALE and TALEN methods suitable for use in the present invention are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein.

According to some embodiments of the present invention, a TALE method comprises silencing or reducing the expression of one or more genes by inhibiting or preventing transcription of the targeted gene(s). For example, a TALE method may include utilizing KRAB-TALEs, wherein the method comprises fusing a transcriptional Kruppel-associated box (KRAB) domain to a DNA binding domain that targets the gene's transcription start site, leading to the inhibition or prevention of transcription of the gene.

According to other embodiments, a TALE method comprises silencing or reducing the expression of one or more genes by introducing mutations in the targeted gene(s). For example, a TALE method may include fusing a nuclease effector domain, such as Fokl, to the TALE DNA binding domain, resulting in a TALEN. Fokl is active as a dimer; hence, the method comprises constructing pairs of TALENs to position the FOKL nuclease domains to adjacent genomic target sites, where they introduce DNA double strand breaks. A double strand break may be completed following correct positioning and dimerization of Fokl. Once the double strand break is introduced, DNA repair can be achieved via two different mechanisms: the high-fidelity homologous recombination pair (HRR) (also known as homology-directed repair or HDR) or the error-prone non-homologous end joining (NHEJ). Repair of double strand breaks via NHEJ preferably results in DNA target site deletions, insertions or substitutions, i.e., NHEJ typically leads to the introduction of small insertions and deletions at the site of the break, often inducing frameshifts that knockout gene function. According to particular embodiments, the TALEN pairs are targeted to the most 5′ exons of the genes, promoting early frame shift mutations or premature stop codons. The genetic mutation(s) introduced by TALEN are preferably permanent. Thus, according to some embodiments, the method comprises silencing or reducing expression of a target gene by utilizing dimerized TALENs to induce a site-specific double strand break that is repaired via error-prone NHEJ, leading to one or more mutations in the targeted gene.

According to other embodiments, a TALEN that is a hybrid protein derived from FokI and AvrXa7, as disclosed in U.S. Patent Publication No. 2011/0201118, may be used in accordance with embodiments of the present invention. This TALEN retains recognition specificity for target nucleotides of AvrXa7 and the double-stranded DNA cleaving activity of FokI. The same methods can be used to prepare other TALEN having different recognition specificity. For example, compact TALENs may be generated by engineering a core TALE scaffold having different sets of RVDs to change the DNA binding specificity and target a specific single dsDNA target sequence. See U.S. Patent Publication No. 2013/0117869. A selection of catalytic domains can be attached to the scaffold to effect DNA processing, which may be engineered to ensure that the catalytic domain is capable of processing DNA near the single dsDNA target sequence when fused to the core TALE scaffold. A peptide linker may also be engineered to fuse the catalytic domain to the scaffold to create a compact TALEN made of a single polypeptide chain that does not require dimerization to target a specific single dsDNA sequence. A core TALE scaffold may also be modified by fusing a catalytic domain, which may be a TAL monomer, to its N-terminus, allowing for the possibility that this catalytic domain might interact with another catalytic domain fused to another TAL monomer, thereby creating a catalytic entity likely to process DNA in the proximity of the target sequences. See U.S. Patent Publication No. 2015/0203871. This architecture allows only one DNA strand to be targeted, which is not an option for classical TALEN architectures.

According to some embodiments of the present invention, conventional RVDs may be used create TALENs that are capable of significantly reducing gene expression. In some embodiments, four RVDs, NI, HD, NN, and NG, are used to target adenine, cytosine, guanine, and thymine, respectively. These conventional RVDs can be used to, for instance, create TALENs targeting the PD-1 gene. Examples of TALENs using conventional RVDs include the T3v1 and T1 TALENs disclosed in Gautron et al., Molecular Therapy: Nucleic Acids December 2017, Vol. 9:312-321 (Gautron), which is incorporated by reference herein. The T3v1 and T1 TALENs target the second exon of the PDCD1 locus where the PD-L1 binding site is located and are able to considerably reduce PD-1 production. In some embodiments, the T1 TALEN does so by using target SEQ ID NO: 127 and the T3v1 TALEN does so by using target SEQ ID NO: 128, as well as those sequences described in Example 1. In some embodiments, the TALENs targeting the PD-1 gene are those described in WO 2013/176915 A1, WO 2014/184744 A1, WO 2014/184741 A1, WO 2018/007263 A1, and WO 2018/073391 A1 including any of the PD-1 TALENs described in Table 10 on pages 62-63 of WO 2013/176915 A1, any of the PD-1 TALENs described in Table 11 on page 78 of WO 2014/184744 A1, any of the PD-1 TALENs described in Table 11 on page 75 of WO 2014/184741 A1, any of the PD-1 TALENs described in Table 3 on pages 48-52 of WO 2018/007263 A1, and any of the PD-1 TALENs described in Table 4 on pages 62-68 and/or in Table 5 on pages 73-99 of WO 2018/073391 A1.

According to other embodiments, TALENs are modified using non-conventional RVDs to improve their activity and specificity for a target gene, such as disclosed in Gautron. Naturally occurring RVDs only cover a small fraction of the potential diversity repertoire for the hypervariable amino acid locations. Non-conventional RVDs provide an alternative to natural RVDs and have novel intrinsic targeting specificity features that can be used to exclude the targeting of off-site targets (sequences within the genome that contain a few mismatches relative to the targeted sequence) by TALEN. Non-conventional RVDs may be identified by generating and screening collections of TALEN containing alternative combinations of amino acids at the two hypervariable amino acid locations at defined positions of an array as disclosed in Juillerat, et al., Scientific Reports 5, Article Number 8150 (2015), which is incorporated by reference herein. Next, non-conventional RVDs may be selected that discriminate between the nucleotides present at the position of mismatches, which can prevent TALEN activity at off-site sequences while still allowing appropriate processing of the target location. The selected non-conventional RVDs may then be used to replace the conventional RVDs in a TALEN. Examples of TALENs where conventional RVDs have been replaced by non-conventional RVDs include the T3v2 and T3v3 PD-1 TALENs produced by Gautron. These TALENs had increased specificity when compared to TALENs using conventional RVDs.

According to other embodiments, TALENs may be specifically designed, which allows higher rates of DSB events within the target cell(s) that are able to target a specific selection of genes. See U.S. Patent Publication No. 2013/0315884. The use of such rare cutting endonucleases increases the chances of obtaining double inactivation of target genes in transfected cells, allowing for the production of engineered cells, such as T-cells. Further, additional catalytic domains can be introduced with the TALEN to increase mutagenesis and enhance target gene inactivation. The TALENs described in U.S. Patent Publication No. 2013/0315884 were successfully used to engineer T-cells to make them suitable for immunotherapy. TALENs may also be used to inactivate various immune checkpoint genes in T-cells, including the inactivation of at least two genes in a single T-cell. See U.S. Patent Publication No. 2016/0120906. Additionally, TALENs may be used to inactivate genes encoding targets for immunosuppressive agents and T-cell receptors, as disclosed in U.S. Patent Publication No. 2018/0021379, which is incorporated by reference herein. Further, TALENs may be used to inhibit the expression of beta 2-microglobulin (B2M) and/or class II major histocompatibility complex transactivator (CIITA), as disclosed in U.S. Patent Publication No. 2019/0010514, which is incorporated by reference herein.

Examples of TALE-nucleases targeting the PD-1 gene are provided in the following table, as well as Table 5 in Example 1, and WO 2018/007263 A1. In these examples, the targeted genomic sequences contain two 17-base pair (bp) long sequences (referred to as half targets, shown in upper case letters) separated by a 15-bp spacer (shown in lower case letters). Each pair of right and left half targets is recognized by the repeat sequences of the corresponding pair of right and left half TALE-nucleases listed in the table. Thus, according to particular embodiments, TALE-nucleases according to the invention recognize and cleave the target sequence selected from the group consisting of: SEQ ID NO: 127 and SEQ ID NO: 128. TALEN sequences and TALEN gene-editing methods are also described in Gautron, discussed above.

TABLE 3
TALEN Sequences

| No. | Target PD-1 Sequence | Repeat Sequence | Half-TALE nuclease |
|---|---|---|---|
| 1 | TTCTCCCCAGCCCTGCT | Repeat PD-1-left | PD-1-left TALEN |
| | cgtggtgaccgaagg | (SEQ ID NO: 129) | (SEQ ID NO: 133) |
| | GGACAACGCCACCTTCA | Repeat PD-1-right | PD-1-right TALEN |
| | (SEQ ID NO: 127) | (SEQ ID NO: 130) | (SEQ ID NO: 134) |
| 2 | TACCTCTGTGGGGCCAT | Repeat PD-1-left | PD-1-left TALEN |
| | ctccctggcccccaa | (SEQ ID NO: 131) | (SEQ ID NO: 135) |
| | GGCGCAGATCAAAGAGA | Repeat PD-1-right | PD-1-right TALEN |
| | (SEQ ID NO: 128) | (SEQ ID NO: 132) | (SEQ ID NO: 136) |

In addition examples of TALE-nucleases targeting the PD-1 gene are provided in WO 2013/176915 A1, WO 2014/184744 A1, WO 2014/184741 A1, WO 2018/007263 A1, and WO 2018/073391 A1 including any of the PD-1 TALENs described in Table 10 on pages 62-63 of WO 2013/176915 A1, any of the PD-1 TALENs described in Table 11 on page 78 of WO 2014/184744 A1, any of the PD-1 TALENs described in Table 11 on page 75 of WO 2014/184741 A1, any of the PD-1 TALENs described in Table 3 on pages 48-52 of WO 2018/007263 A1, and any of the PD-1 TALENs described in Table 4 on pages 62-68 and/or in Table 5 on pages 73-99 of WO 2018/073391 A1.

Examples of systems, methods, and compositions for altering the expression of a target gene sequence by a TALE method, and which may be used in accordance with embodiments of the present invention, are described in U.S. Pat. No. 8,586,526, which is incorporated by reference herein. These disclosed examples include the use of a non-naturally occurring DNA-binding polypeptide that has two or more TALE-repeat units containing a repeat RVD, an N-cap polypeptide made of residues of a TALE protein, and a C-cap polypeptide made of a fragment of a full length C-terminus region of a TALE protein.

Examples of TALEN designs and design strategies, activity assessments, screening strategies, and methods that can be used to efficiently perform TALEN-mediated gene integration and inactivation, and which may be used in accordance with embodiments of the present invention, are described in Valton, et al., Methods, 2014, 69, 151-170, which is incorporated by reference herein.

### IV. TIL Manufacturing Processes—2A (Gen 2)

An exemplary process for production and expansion of the genetically modified TILs of the invention is depicted in FIG. 7, wherein the expanded TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. As discussed herein, the present invention can include a step relating to the restimulation of the genetically modified cryopreserved TILs to increase their metabolic activity and thus relative health prior to transplant into a patient, and methods of testing said metabolic health. As generally outlined herein, TILs are generally taken from a patient sample, genetically modified, and manipulated to expand their number prior to transplant into a patient. In some embodiments, these genetically modified TILs may be cryopreserved. Once thawed, they may also be restimulated to increase their metabolism prior to infusion into a patient.

In some embodiments, the first expansion in the preparation of these genetically modified TILs (including processes referred to as the preREP as well as processes shown in FIG. 7 as Step B1) is shortened to 3 to 14 days and the second expansion (including processes referred to as the REP as well as processes shown in FIG. 7 as Step C) is shorted to 7 to 14 days, as discussed in detail below as well as in the examples and FIG. 7, wherein the expanded TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the first expansion in the preparation of the genetically modified TILs (for example, an expansion described as Step B1 in FIG. 7) is shortened to 11 days and the second expansion (for example, an expansion as described in Step C in FIG. 7) is shortened to 11 days, wherein the expanded TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the combination of the first expansion and second expansion (for example, expansions described as Step B1 and Step C in FIG. 7) is shortened to 22 days, as discussed in detail below and in the examples and FIG. 7, wherein the expanded TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1.

The “Step” Designations A, B, C, etc., below are in reference to FIG. 7 and in reference to certain embodiments described herein. The ordering of the Steps below and in FIG. 7 is exemplary and any combination or order of steps, as well as additional steps, repetition of steps, and/or omission of steps is contemplated by the present application and the methods disclosed herein.

### A. STEP A: Obtain Patient Tumor Sample

In general, TILs are initially obtained from a patient tumor sample (“primary TILs”) and then expanded into a larger population for further manipulation as described herein, optionally cryopreserved, restimulated as outlined herein and optionally evaluated for phenotype and metabolic parameters as an indication of TIL health, wherein the expanded TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1.

A patient tumor sample may be obtained using methods known in the art, generally via surgical resection, needle biopsy, core biopsy, small biopsy, or other means for obtaining a sample that contains a mixture of tumor and TIL cells. In some embodiments, multilesional sampling is used. In some embodiments, surgical resection, needle biopsy, core biopsy, small biopsy, or other means for obtaining a sample that contains a mixture of tumor and TIL cells includes multilesional sampling (i.e., obtaining samples from one or more tumor cites and/or locations in the patient, as well as one or more tumors in the same location or in close proximity). In general, the tumor sample may be from any solid tumor, including primary tumors, invasive tumors or metastatic tumors. The tumor sample may also be a liquid tumor, such as a tumor obtained from a hematological malignancy. The solid tumor may be of skin tissue. In some embodiments, useful TILs are obtained from a melanoma.

Once obtained, the tumor sample is generally fragmented using sharp dissection into small pieces of between 1 to about 8 mm³, with from about 2-3 mm³ being particularly useful. In some embodiments, the TILs are cultured from these fragments using enzymatic tumor digests. Such tumor digests may be produced by incubation in enzymatic media (e.g., Roswell Park Memorial Institute (RPMI) 1640 buffer, 2 mM glutamate, 10 meg/mL gentamicine, 30 units/mL of DNase and 1.0 mg/mL of collagenase) followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods of expanding TILs or methods treating a cancer.

As indicated above, in some embodiments, the TILs are derived from solid tumors. In some embodiments, the solid tumors are not fragmented. In some embodiments, the solid tumors are not fragmented and are subjected to enzymatic digestion as whole tumors. In some embodiments, the tumors are digested in in an enzyme mixture comprising collagenase, DNase, and neutral protease. In some embodiments, the tumors are digested in in an enzyme mixture comprising collagenase, DNase, and neutral protease for 1-2 hours. In some embodiments, the tumors are digested in in an enzyme mixture comprising collagenase, DNase, and neutral protease for 1-2 hours at 37° C., 5% CO₂. In some embodiments, the tumors are digested in in an enzyme mixture comprising collagenase, DNase, and neutral protease for 1-2 hours at 37° C., 5% CO₂ with rotation. In some embodiments, the tumors are digested overnight with constant rotation. In some embodiments, the tumors are digested overnight at 37° C., 5% CO₂ with constant rotation. In some embodiments, the whole tumor is combined with the enzymes to form a tumor digest reaction mixture.

In some embodiments, the tumor is reconstituted with the lyophilized enzymes in a sterile buffer. In some embodiments, the buffer is sterile HBSS.

In some embodiments, the enzyme mixture comprises collagenase. In some embodiments, the collagenase is collagenase IV. In some embodiments, the working stock for the collagenase is a 100 mg/ml 10× working stock.

In some embodiments, the enzyme mixture comprises DNAse. In some embodiments, the working stock for the DNAse is a 10,000 IU/ml 10× working stock.

In some embodiments, the enzyme mixture comprises hyaluronidase. In some embodiments, the working stock for the hyaluronidase is a 10-mg/ml 10× working stock.

In some embodiments, the enzyme mixture comprises 10 mg/ml collagenase, 1000 IU/ml DNAse, and 1 mg/ml hyaluronidase.

In some embodiments, the enzyme mixture comprises 10 mg/ml collagenase, 500 IU/ml DNAse, and 1 mg/ml hyaluronidase.

In some embodiments, the enzyme mixture comprises neutral protease. In some embodiments, the working stock for the neutral protease is reconstituted at a concentration of 175 DMC U/mL.

In some embodiments, the enzyme mixture comprises neutral protease, DNase, and collagenase.

In some embodiments, the enzyme mixture comprises 10 mg/ml collagenase, 1000 IU/ml DNase, and 0.31 DMC U/ml neutral protease. In some embodiments, the enzyme mixture comprises 10 mg/ml collagenase, 500 IU/ml DNase, and 0.31 DMC U/ml neutral protease.

In general, the harvested cell suspension is called a “primary cell population” or a “freshly harvested” cell population.

In some embodiments, fragmentation includes physical fragmentation, including for example, dissection as well as digestion. In some embodiments, the fragmentation is physical fragmentation. In some embodiments, the fragmentation is dissection. In some embodiments, the fragmentation is by digestion. In some embodiments, TILs can be initially cultured from enzymatic tumor digests and tumor fragments obtained from patients. In some embodiments, TILs can be initially cultured from enzymatic tumor digests and tumor fragments obtained from patients prior to genetic modification via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1.

In some embodiments, where the tumor is a solid tumor, the tumor undergoes physical fragmentation after the tumor sample is obtained in, for example, Step A (as provided in FIG. 7). In some embodiments, the fragmentation occurs before cryopreservation. In some embodiments, the fragmentation occurs after cryopreservation. In some embodiments, the fragmentation occurs after obtaining the tumor and in the absence of any cryopreservation. In some embodiments, the tumor is fragmented and 10, 20, 30, 40, 50, 60, 70, 80, 90, 100 or more fragments or pieces are placed in each container for the first expansion. In some embodiments, the tumor is fragmented and 30 or 40 fragments or pieces are placed in each container for the first expansion. In some embodiments, the tumor is fragmented and 40 fragments or pieces are placed in each container for the first expansion. In some embodiments, the multiple fragments comprise about 4 to about 50 fragments, wherein each fragment has a volume of about 27 mm³. In some embodiments, the multiple fragments comprise about 30 to about 60 fragments with a total volume of about 1300 mm³ to about 1500 mm³. In some embodiments, the multiple fragments comprise about 50 fragments with a total volume of about 1350 mm³. In some embodiments, the multiple fragments comprise about 50 fragments with a total mass of about 1 gram to about 1.5 grams. In some embodiments, the multiple fragments comprise about 4 fragments. In some embodiments, the multiple fragments comprise about to about 100 fragments.

In some embodiments, the TILs are obtained from tumor fragments. In some embodiments, the tumor fragment is obtained by sharp dissection. In some embodiments, the tumor fragment is between about 1 mm³ and 10 mm³. In some embodiments, the tumor fragment is between about 1 mm³ and 8 mm³. In some embodiments, the tumor fragment is about 1 mm³. In some embodiments, the tumor fragment is about 2 mm³. In some embodiments, the tumor fragment is about 3 mm³. In some embodiments, the tumor fragment is about 4 mm³. In some embodiments, the tumor fragment is about 5 mm³. In some embodiments, the tumor fragment is about 6 mm³. In some embodiments, the tumor fragment is about 7 mm³. In some embodiments, the tumor fragment is about 8 mm³. In some embodiments, the tumor fragment is about 9 mm³. In some embodiments, the tumor fragment is about 10 mm³. In some embodiments, the tumors are 1-4 mmx 1-4 mm×1-4 mm. In some embodiments, the tumors are 1 mmx 1 mm×1 mm. In some embodiments, the tumors are 2 mmx 2 mm×2 mm. In some embodiments, the tumors are 3 mmx 3 mm×3 mm. In some embodiments, the tumors are 4 mmx 4 mm×4 mm.

In some embodiments, the tumors are resected in order to minimize the amount of hemorrhagic, necrotic, and/or fatty tissues on each piece. In some embodiments, the tumors are resected in order to minimize the amount of hemorrhagic tissue on each piece. In some embodiments, the tumors are resected in order to minimize the amount of necrotic tissue on each piece. In some embodiments, the tumors are resected in order to minimize the amount of fatty tissue on each piece.

In some embodiments, the tumor fragmentation is performed in order to maintain the tumor internal structure. In some embodiments, the tumor fragmentation is performed without preforming a sawing motion with a scalpel. In some embodiments, the TILs are obtained from tumor digests. In some embodiments, tumor digests were generated by incubation in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, CA). After placing the tumor in enzyme media, the tumor can be mechanically dissociated for approximately 1 minute. The solution can then be incubated for 30 minutes at 37° C. in 5% CO₂ and it then mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue were present, 1 or 2 additional mechanical dissociations were applied to the sample, with or without 30 additional minutes of incubation at 37° C. in 5% CO₂. In some embodiments, at the end of the final incubation if the cell suspension contained a large number of red blood cells or dead cells, a density gradient separation using Ficoll can be performed to remove these cells.

In some embodiments, the harvested cell suspension prior to the first expansion step is called a “primary cell population” or a “freshly harvested” cell population.

In some embodiments, cells can be optionally frozen after sample harvest and stored frozen prior to entry into the expansion described in Step B, which is described in further detail below, as well as exemplified in FIG. 7.

### B. STEP B1: First Expansion

In some embodiments, the present methods provide for obtaining young TILs, which are capable of increased replication cycles upon administration to a subject/patient and as such may provide additional therapeutic benefits over older TILs (i.e., TILs which have further undergone more rounds of replication prior to administration to a subject/patient).

Features of young TILs have been described in the literature, for example Donia, at al., Scandinavian Journal of Immunology, 75:157-167 (2012); Dudley et al., Clin Cancer Res, 16:6122-6131 (2010); Huang et al., J Immunother, 28(3):258-267 (2005); Besser et al., Clin Cancer Res, 19(17):OF1-OF9 (2013); Besser et al., J Immunother 32:415-423 (2009); Robbins, et al., J Immunol 2004; 173:7125-7130; Shen et al., J Immunother, 30:123-129 (2007); Zhou, et al., J Immunother, 28:53-62 (2005); and Tran, et al., J Immunother, 31:742-751 (2008), all of which are incorporated herein by reference in their entireties.

The diverse antigen receptors of T and B lymphocytes are produced by somatic recombination of a limited, but large number of gene segments. These gene segments: V (variable), D (diversity), J (joining), and C (constant), determine the binding specificity and downstream applications of immunoglobulins and T-cell receptors (TCRs). The present invention provides a method for generating expanded TILs which exhibit and increase the T-cell repertoire diversity, wherein the expanded TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the expanded TILs obtained by the present method exhibit an increase in the T-cell repertoire diversity, wherein the expanded TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the expanded TILs (wherein the expanded TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1) obtained by the present method exhibit an increase in the T-cell repertoire diversity as compared to freshly harvested TILs and/or TILs prepared using other methods than those provide herein including for example, methods other than those embodied in FIG. 7. In some embodiments, the TILs obtained in the first expansion exhibit an increase in the T-cell repertoire diversity, wherein the expanded TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the increase in diversity is an increase in the immunoglobulin diversity and/or the T-cell receptor diversity. In some embodiments, the diversity is in the immunoglobulin is in the immunoglobulin heavy chain. In some embodiments, the diversity is in the immunoglobulin is in the immunoglobulin light chain. In some embodiments, the diversity is in the T-cell receptor. In some embodiments, the diversity is in one of the T-cell receptors selected from the group consisting of alpha, beta, gamma, and delta receptors. In some embodiments, there is an increase in the expression of T-cell receptor (TCR) alpha and/or beta. In some embodiments, there is an increase in the expression of T-cell receptor (TCR) alpha. In some embodiments, there is an increase in the expression of T-cell receptor (TCR) beta. In some embodiments, there is an increase in the expression of TCRab (i.e., TCRα/β).

After dissection or digestion of tumor fragments, for example such as described in Step A of FIG. 7, the resulting cells are cultured in serum containing IL-2 under conditions that favor the growth of TILs over tumor and other cells. In some embodiments, the tumor digests are incubated in 2 mL wells in media comprising inactivated human AB serum with 6000 IU/mL of IL-2. This primary cell population is cultured for a period of days, generally from 3 to 14 days, resulting in a bulk TIL population, generally about 1×10⁸ bulk TIL cells, wherein the expanded TILs have been or will be genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, this primary cell population is cultured for a period of 7 to 14 days, resulting in a bulk TIL population, generally about 1×10⁸ bulk TIL cells, wherein the expanded TILs have been or will be genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, this primary cell population is cultured for a period of 10 to 14 days, resulting in a bulk TIL population, generally about 1×10⁸ bulk TIL cells. In some embodiments, this primary cell population is cultured for a period of about 11 days, resulting in a bulk TIL population, generally about 1×10⁸ bulk TIL cells, wherein the expanded TILs have been or will be genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1.

In some embodiments, expansion of TILs may be performed using an initial bulk TIL expansion step (for example such as those described in Step B1 of FIG. 7, which can include processes referred to as pre-REP) as described below and herein, wherein the expanded TILs have been or will be genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. The TILs obtained from this process may be optionally characterized for phenotypic characteristics and metabolic parameters as described herein.

In embodiments where TIL cultures are initiated in 24-well plates, for example, using Costar 24-well cell culture cluster, flat bottom (Corning Incorporated, Corning, NY, each well can be seeded with 1×10⁶ tumor digest cells or one tumor fragment in 2 mL of complete medium (CM) with IL-2 (6000 IU/mL; Chiron Corp., Emeryville, CA), wherein the expanded TILs have been or will be genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the tumor fragment is between about 1 mm³ and 10 mm³.

In some embodiments, the first expansion culture medium is referred to as “CM”, an abbreviation for culture media. In some embodiments, CM for Step B consists of RPMI 1640 with GlutaMAX, supplemented with 10% human AB serum, 25 mM Hepes, and 10 mg/mL gentamicin. In embodiments where cultures are initiated in gas-permeable flasks with a 40 mL capacity and a 10 cm² gas-permeable silicon bottom (for example, G-Rex10; Wilson Wolf Manufacturing, New Brighton, MN, each flask may be loaded with 10-40×10⁶ viable tumor digest cells or 5-30 tumor fragments in 10-40 mL of CM with IL-2. Both the G-Rex10 and 24-well plates may be incubated in a humidified incubator at 37° C. in 5% CO₂ and 5 days after culture initiation, half the media may be removed and replaced with fresh CM and IL-2 and after day 5, half the media may be changed every 2-3 days.

After preparation of the tumor fragments, the resulting cells (i.e., fragments) are cultured in serum containing IL-2 under conditions that favor the growth of TILs over tumor and other cells, wherein the TILs whose growth is favored have been or will be genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the tumor digests are incubated in 2 mL wells in media comprising inactivated human AB serum (or, in some cases, as outlined herein, in the presence of aAPC cell population) with 6000 IU/mL of IL-2. This primary cell population is cultured for a period of days, generally from 10 to 14 days, resulting in a bulk TIL population, generally about 1×10⁸ bulk TIL cells. In some embodiments, the growth media during the first expansion comprises IL-2 or a variant thereof. In some embodiments, the IL is recombinant human IL-2 (rhIL-2). In some embodiments the IL-2 stock solution has a specific activity of 20-30×10⁶ IU/mg for a 1 mg vial. In some embodiments the IL-2 stock solution has a specific activity of 20×10⁶ IU/mg for a 1 mg vial. In some embodiments the IL-2 stock solution has a specific activity of 25×10⁶ IU/mg for a 1 mg vial. In some embodiments the IL-2 stock solution has a specific activity of 30×10⁶ IU/mg for a 1 mg vial. In some embodiments, the IL-2 stock solution has a final concentration of 4-8×10⁶ IU/mg of IL-2. In some embodiments, the IL-2 stock solution has a final concentration of 5-7×10⁶ IU/mg of IL-2. In some embodiments, the IL-2 stock solution has a final concentration of 6×10⁶ IU/mg of IL-2. In some embodiments, the IL-2 stock solution is prepare as described in Example 5. In some embodiments, the first expansion culture media comprises about 10,000 IU/mL of IL-2, about 9,000 IU/mL of IL-2, about 8,000 IU/mL of IL-2, about 7,000 IU/mL of IL-2, about 6000 IU/mL of IL-2 or about 5,000 IU/mL of IL-2. In some embodiments, the first expansion culture media comprises about 9,000 IU/mL of IL-2 to about 5,000 IU/mL of IL-2. In some embodiments, the first expansion culture media comprises about 8,000 IU/mL of IL-2 to about 6,000 IU/mL of IL-2. In some embodiments, the first expansion culture media comprises about 7,000 IU/mL of IL-2 to about 6,000 IU/mL of IL-2. In some embodiments, the first expansion culture media comprises about 6,000 IU/mL of IL-2. In some embodiments, the cell culture medium further comprises IL-2. In some embodiments, the cell culture medium comprises about 3000 IU/mL of IL-2. In some embodiments, the cell culture medium further comprises IL-2. In some embodiments, the cell culture medium comprises about 3000 IU/mL of IL-2. In some embodiments, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In some embodiments, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or about 8000 IU/mL of IL-2.

In some embodiments, the cell culture medium comprises OKT-3 antibody. In some embodiments, the cell culture medium comprises about 30 ng/mL of OKT-3 antibody. In some embodiments, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, or about 1 μg/mL of OKT-3 antibody. In some embodiments, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody. In some embodiments, the cell culture medium does not comprise OKT-3 antibody. In some embodiments, the OKT-3 antibody is muromonab (see Table 1).

In some embodiments, the cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 μg/mL and 100 μg/mL. In some embodiments, the TNFRSF agonist is added at a concentration sufficient to achieve a concentration in the cell culture medium of between 20 μg/mL and 40 μg/mL.

In some embodiments, in addition to one or more TNFRSF agonists, the cell culture medium further comprises IL-2 at an initial concentration of about 3000 IU/mL and OKT-3 antibody at an initial concentration of about 30 ng/mL, and wherein the one or more TNFRSF agonists comprises a 4-1BB agonist.

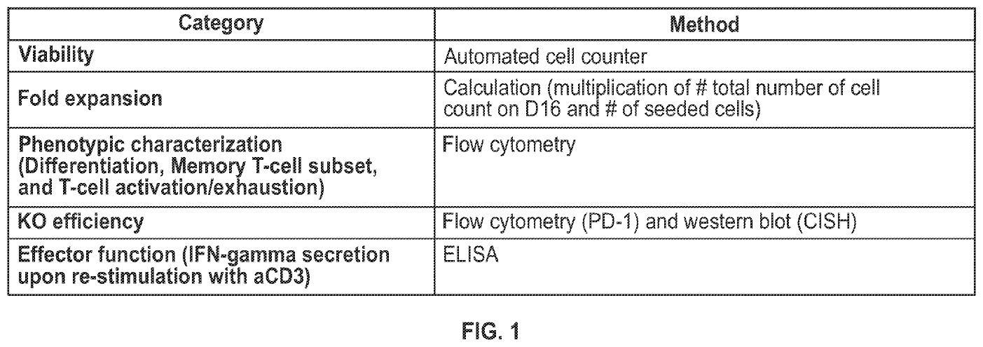

In some embodiments, the first expansion culture medium is referred to as “CM”, an abbreviation for culture media. In some embodiments, it is referred to as CM1 (culture medium 1). In some embodiments, CM consists of RPMI 1640 with GlutaMAX, supplemented with 10% human AB serum, 25 mM Hepes, and 10 mg/mL gentamicin. In embodiments where cultures are initiated in gas-permeable flasks with a 40 mL capacity and a 10 cm² gas-permeable silicon bottom (for example, G-Rex10; Wilson Wolf Manufacturing, New Brighton, MN) (FIG. 1), each flask may be loaded with 10-40×10⁶ viable tumor digest cells or 5-30 tumor fragments in 10-40 mL of CM with IL-2. Both the G-Rex10 and 24-well plates may be incubated in a humidified incubator at 37° C. in 5% CO₂ and 5 days after culture initiation, half the media may be removed and replaced with fresh CM and IL-2 and after day 5, half the media may be changed every 2-3 days. In some embodiments, the CM is the CM1 described in the Examples, see, Example 1. In some embodiments, the first expansion occurs in an initial cell culture medium or a first cell culture medium. In some embodiments, the initial cell culture medium or the first cell culture medium comprises IL-2.

In some embodiments, the first expansion (including processes such as for example those described in Step B1 of FIG. 7, which can include those sometimes referred to as the pre-REP) process is shortened to 3-14 days, as discussed in the examples and figures. In some embodiments, the first expansion (including processes such as for example those described in Step B1 of FIG. 7, which can include those sometimes referred to as the pre-REP) is shortened to 7 to 14 days, as discussed in the Examples and shown in the expansion described in Step B1 of FIG. 7. In some embodiments, the first expansion of Step B1 is shortened to 10-14 days. In some embodiments, the first expansion is shortened to 11 days, as discussed in, for example, an expansion as described in Step B1 of FIG. 7.

In some embodiments, the first TIL expansion can proceed for 1 day, 2 days, 3 days, 4 days, 5 days, 6 days, 7 days, 8 days, 9 days, 10 days, 11 days, 12 days, 13 days, or 14 days, wherein the expanded TILs have been or will be genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the first TIL expansion can proceed for 1 day to 14 days. In some embodiments, the first TIL expansion can proceed for 2 days to 14 days. In some embodiments, the first TIL expansion can proceed for 3 days to 14 days. In some embodiments, the first TIL expansion can proceed for 4 days to 14 days. In some embodiments, the first TIL expansion can proceed for 5 days to 14 days. In some embodiments, the first TIL expansion can proceed for 6 days to 14 days. In some embodiments, the first TIL expansion can proceed for 7 days to 14 days. In some embodiments, the first TIL expansion can proceed for 8 days to 14 days. In some embodiments, the first TIL expansion can proceed for 9 days to 14 days. In some embodiments, the first TIL expansion can proceed for 10 days to 14 days. In some embodiments, the first TIL expansion can proceed for 11 days to 14 days. In some embodiments, the first TIL expansion can proceed for 12 days to 14 days. In some embodiments, the first TIL expansion can proceed for 13 days to 14 days. In some embodiments, the first TIL expansion can proceed for 14 days. In some embodiments, the first TIL expansion can proceed for 1 day to 11 days. In some embodiments, the first TIL expansion can proceed for 2 days to 11 days. In some embodiments, the first TIL expansion can proceed for 3 days to 11 days. In some embodiments, the first TIL expansion can proceed for 4 days to 11 days. In some embodiments, the first TIL expansion can proceed for 5 days to 11 days. In some embodiments, the first TIL expansion can proceed for 6 days to 11 days. In some embodiments, the first TIL expansion can proceed for 7 days to 11 days. In some embodiments, the first TIL expansion can proceed for 8 days to 11 days. In some embodiments, the first TIL expansion can proceed for 9 days to 11 days. In some embodiments, the first TIL expansion can proceed for 10 days to 11 days. In some embodiments, the first TIL expansion can proceed for 11 days.

In some embodiments, the first expansion, for example, Step B1 according to FIG. 7, is performed in a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100. In some embodiments, the closed system bioreactor is a single bioreactor.

### C. STEP B2: Activation

In some embodiments, after the pre-REP step (Step B2 in FIG. 7) the TILs are activated by adding OKT-3 to the culture medium and culturing for about 1 to 3 days, wherein the TILs have been or will be genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the activation step (for example, Step B2 in FIG. 7) is performed for about 2 days.

In some embodiments, the activation step (for example, Step B2 in FIG. 7) is performed by culturing the TILs in the presence of 300 ng/ml OKT-3 for about 1 to 3 days.

In some embodiments, the cell culture medium in the activation step (for example, Step B2 in FIG. 7) comprises about 300 ng/mL of OKT-3 antibody. In some embodiments, the cell culture medium in the activation step (for example, Step B2 in FIG. 7) comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 300 ng/ml, about 400 ng/ml, about 500 ng/mL, about 600 ng/ml, about 700 ng/ml, about 800 ng/ml, about 900 ng/ml, or about 1 μg/mL of OKT-3 antibody. In some embodiments, the cell culture medium in the activation step (for example, Step B2 in FIG. 7) comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, between 50 ng/mL and 100 ng/mL, between 100 ng/ml and 500 ng/ml, between 200 ng/ml and 400 ng/ml, between 250 ng/ml and 350 ng/ml, or between 275 ng/ml and 325 ng/ml of OKT-3 antibody. In some embodiments, the OKT-3 antibody is muromonab (see Table 1).

In some embodiments, the activation step (for example, Step B2 in FIG. 7) is performed by adding OKT-3 to the TILs in culture without opening the system.

### D. STEP B3: TALEN Gene Modification Step

In some embodiments, the activation step (for example, Step B3 in FIG. 7) is followed by a step of genetically modifying TILs by introducing into the TILs nucleic acids, such as mRNAs, encoding one or more TALE-nucleases to selectively inactivate by DNA cleavage a gene encoding CISH, wherein the one or more TALE-nucleases comprise the TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1 (for example, Step B3 in FIG. 8).

In some embodiments, the TALEN gene modification step (for example, Step B3 in FIG. 7) is performed by genetically modifying the TILs obtained from the activation step (for example, Step B2 in FIG. 7) by electroporation of TILs with nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by electroporation of TILs with nucleic acids, such as mRNAs, encoding one or more TALE-nucleases to selectively inactivate by DNA cleavage a gene encoding PD-1.

In some embodiments, the TALEN gene modification step (for example, Step B3 in FIG. 7) is performed by genetically modifying the TILs obtained from the activation step (for example, Step B2 in FIG. 7) by electroporation of TILs with nucleic acids, such as mRNAs, encoding one or more TALE-nucleases to selectively inactivate by DNA cleavage a gene encoding CISH, wherein the one or more TALE-nucleases comprise a TALE-nuclease having an amino acid sequence comprising SEQ ID NO: 164 and a TALE-nuclease having an amino acid sequence comprising SEQ ID NO: 166, and optionally by electroporation of TILs with nucleic acids, such as mRNAs, encoding one or more TALE-nucleases to selectively inactivate by DNA cleavage a gene encoding PD-1.

In some embodiments, the TALEN gene modification step (for example, Step B3 in FIG. 7) is performed by genetically modifying the TILs obtained from the activation step (for example, Step B2 in FIG. 7) by electroporation of TILs with nucleic acids, such as mRNAs, encoding one or more TALE-nucleases to selectively inactivate by DNA cleavage a gene encoding CISH, wherein the one or more TALE-nucleases comprise a TALE-nuclease having an amino acid sequence comprising SEQ ID NO: 165 and a TALE-nuclease having an amino acid sequence comprising SEQ ID NO: 167, and optionally by electroporation of TILs with nucleic acids, such as mRNAs, encoding one or more TALE-nucleases to selectively inactivate by DNA cleavage a gene encoding PD-1.

In some embodiments, the TALEN gene modification step (for example, Step B3 in FIG. 7) is performed by genetically modifying the TILs obtained from the activation step (for example, Step B2 in FIG. 7) by electroporation of TILs with nucleic acids, such as mRNAs, encoding one or more TALE-nucleases to selectively inactivate by DNA cleavage a gene encoding CISH, wherein the one or more TALE-nucleases comprise a TALE-nuclease having an amino acid sequence comprising SEQ ID NO: 164 and a TALE-nuclease having an amino acid sequence comprising SEQ ID NO: 167, and optionally by electroporation of TILs with nucleic acids, such as mRNAs, encoding one or more TALE-nucleases to selectively inactivate by DNA cleavage a gene encoding PD-1.

In some embodiments, the TALEN gene modification step (for example, Step B3 in FIG. 7) is performed by genetically modifying the TILs obtained from the activation step (for example, Step B2 in FIG. 7) by electroporation of TILs with nucleic acids, such as mRNAs, encoding one or more TALE-nucleases to selectively inactivate by DNA cleavage a gene encoding CISH, wherein the desired TALE-nucleases comprise a TALE-nuclease having an amino acid sequence comprising SEQ ID NO: 165 and a TALE-nuclease having an amino acid sequence comprising SEQ ID NO: 166, and optionally by electroporation of TILs with nucleic acids, such as mRNAs, encoding one or more TALE-nucleases to selectively inactivate by DNA cleavage a gene encoding PD-1.

In some embodiments, the TALEN gene modification step described in any of the preceding paragraphs as applicable above is modified such that the nucleic acids, such as mRNAs, used for electroporation of TILs include nucleic acids, such as mRNAs, encoding TALE-nucleases to selectively inactivate by DNA cleavage a gene encoding PD-1.

In some embodiments, the TALEN gene modification step described in any of the preceding paragraphs as applicable above is modified such that the nucleic acids, such as mRNAs, used for electroporation of TILs include nucleic acids, such as mRNAs, encoding a TALE-nuclease having an amino acid sequence comprising SEQ ID NO: 170, and further include nucleic acids, such as mRNAs, encoding a TALE-nuclease having an amino acid sequence comprising SEQ ID NO: 172.

In some embodiments, the TALEN gene modification step described in any of the preceding paragraphs as applicable above is modified such that the nucleic acids, such as mRNAs, that encode the one or more TALE-nucleases to selectively inactivate the CISH gene, the nucleic acids, such as mRNAs, that encode the one or more TALE-nucleases to selectively inactivate the PD-1 gene, and an electroporation buffer are admixed together, and the TILs are subjected to a single electroporation step in the presence of the admixture.

Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. Other electroporation methods known in the art, such as those described in U.S. Pat. Nos. 5,019,034; 5,128,257; 5,137,817; 5,173,158; 5,232,856; 5,273,525; 5,304,120; 5,318,514; 6,010,613 and 6,078,490, the disclosures of which are incorporated by reference herein, may be used. In some embodiments, the electroporation method is a sterile electroporation method. In some embodiments, the electroporation method is a pulsed electroporation method. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse amplitude. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse width. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein a first pulse interval for a first set of two of the at least three pulses is different from a second pulse interval for a second set of two of the at least three pulses. In some embodiments, the electroporation method is a pulsed electroporation method comprising the steps of treating TILs with pulsed electrical fields to induce pore formation in the TILs, comprising the step of applying a sequence of at least three DC electrical pulses, having field strengths equal to or greater than 100 V/cm, to TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses, such that induced pores are sustained for a relatively long period of time, and such that viability of the TILs is maintained. In some embodiments, a method of genetically modifying a population of TILs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In some embodiments, a method of genetically modifying a population of TILs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In some embodiments, a method of genetically modifying a population of TILs includes the step of transfection using methods described in U.S. Pat. Nos. 5,766,902; 6,025,337; 6,410,517; 6,475,994; and 7,189,705; the disclosures of each of which are incorporated by reference herein.

In some embodiments of the present invention, electroporation is used for delivery of the desired TALEN-encoding nucleic acid, including TALEN-encoding RNAs and/or DNAs. In some embodiments of the present invention, the electroporation system is a flow electroporation system. An example of a suitable flow electroporation system suitable for use with some embodiments of the present invention is the commercially-available MaxCyte STX system. There are several alternative commercially-available electroporation instruments which may be suitable for use with the present invention, such as the AgilePulse system or ECM 830 available from BTX-Harvard Apparatus, Cellaxess Elektra (Cellectricon), Nucleofector (Lonza/Amaxa), GenePulser MXcell (BIORAD), iPorator-96 (Primax) or siPORTer96 (Ambion). In some embodiments of the present invention, the electroporation system forms a closed, sterile system with the remainder of the TIL expansion method. In some embodiments of the present invention, the electroporation system is a pulsed electroporation system as described herein, and forms a closed, sterile system with the remainder of the TIL expansion method.

### E. STEP B4: Rest Step

In some embodiments, the gene modification step (for example, Step B3 in FIG. 7) is followed by a step of resting the TILs (for example, Step B4 in FIG. 8), wherein the resting TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the TILs are rested for about 1 day. In some embodiments, immediately after the electroporation in the gene modification step (for example, Step B3 in FIG. 7), the TILs are rested for about 16 hours. In some embodiments, immediately after the electroporation in the gene modification step (for example, Step B3 in FIG. 7), the TILs are resuspended in CM1 media and incubated at 37° C. for an hour, followed by 30° C. for 15 hours.

### F. STEP C: First Expansion to Second Expansion Transition

In some cases, the genetically modified TIL population obtained from the first expansion, including for example the TIL population obtained from, for example, Step B1 as indicated in FIG. 7, can be cryopreserved immediately, using the protocols discussed herein below, wherein the genetic modification comprises TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. Alternatively, the TIL population obtained from the first expansion, referred to as the second TIL population, can be subjected to genetic modification without an interim cryopreservation, as described above, followed by a second expansion (which can include expansions sometimes referred to as REP) and then cryopreserved as discussed below, wherein the genetic modification comprises TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1.

### G. STEP D: Second Expansion

In some embodiments, the TIL cell population is expanded in number after initial bulk processing, pre-REP expansion, and genetic modification, for example, after Step A and Step B, and the transition referred to as Step C, as indicated in FIG. 7, wherein the expanded TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. This further expansion is referred to herein as the second expansion, which can include expansion processes generally referred to in the art as a rapid expansion process (REP; as well as processes as indicated in Step D of FIG. 7). The second expansion is generally accomplished using a culture media comprising a number of components, including feeder cells, a cytokine source, and an anti-CD3 antibody, in a gas-permeable container.

In some embodiments, the second expansion or second TIL expansion (which can include expansions sometimes referred to as REP; as well as processes as indicated in Step D of FIG. 7) of TIL can be performed using any TIL flasks or containers known by those of skill in the art, wherein the expanded TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the second TIL expansion can proceed for 7 days, 8 days, 9 days, 10 days, 11 days, 12 days, 13 days, or 14 days. In some embodiments, the second TIL expansion can proceed for about 7 days to about 14 days. In some embodiments, the second TIL expansion can proceed for about 8 days to about 14 days. In some embodiments, the second TIL expansion can proceed for about 9 days to about 14 days. In some embodiments, the second TIL expansion can proceed for about 10 days to about 14 days. In some embodiments, the second TIL expansion can proceed for about 11 days to about 14 days. In some embodiments, the second TIL expansion can proceed for about 12 days to about 14 days. In some embodiments, the second TIL expansion can proceed for about 13 days to about 14 days. In some embodiments, the second TIL expansion can proceed for about 14 days.

In some embodiments, the second expansion can be performed in a gas permeable container using the methods of the present disclosure (including for example, expansions referred to as REP; as well as processes as indicated in Step D of FIG. 7). For example, TILs can be rapidly expanded using non-specific T-cell receptor stimulation in the presence of interleukin-2 (IL-2) or interleukin-15 (IL-15). The non-specific T-cell receptor stimulus can include, for example, an anti-CD3 antibody, such as about 30 ng/ml of OKT3, a mouse monoclonal anti-CD3 antibody (commercially available from Ortho-McNeil, Raritan, NJ or Miltenyi Biotech, Auburn, CA) or UHCT-1 (commercially available from BioLegend, San Diego, CA, USA). TILs can be expanded to induce further stimulation of the TILs in vitro by including one or more antigens during the second expansion, including antigenic portions thereof, such as epitope(s), of the cancer, which can be optionally expressed from a vector, such as a human leukocyte antigen A2 (HLA-A2) binding peptide, e.g., 0.3 μM MART-1:26-35 (27 L) or gpl 00:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. TIL may also be rapidly expanded by re-stimulation with the same antigen(s) of the cancer pulsed onto HLA-A2-expressing antigen-presenting cells. Alternatively, the TILs can be further re-stimulated with, e.g., example, irradiated, autologous lymphocytes or with irradiated HLA-A2+ allogeneic lymphocytes and IL-2. In some embodiments, the re-stimulation occurs as part of the second expansion. In some embodiments, the second expansion occurs in the presence of irradiated, autologous lymphocytes or with irradiated HLA-A2+ allogeneic lymphocytes and IL-2.

In some embodiments, the cell culture medium further comprises IL-2. In some embodiments, the cell culture medium comprises about 3000 IU/mL of IL-2. In some embodiments, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In some embodiments, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In some embodiments, the cell culture medium comprises OKT-3 antibody. In some embodiments, the cell culture medium comprises about 30 ng/mL of OKT-3 antibody. In some embodiments, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, or about 1 μg/mL of OKT-3 antibody. In some embodiments, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody. In some embodiments, the cell culture medium does not comprise OKT-3 antibody. In some embodiments, the OKT-3 antibody is muromonab.

In some embodiments, the cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 μg/mL and 100 μg/mL. In some embodiments, the TNFRSF agonist is added at a concentration sufficient to achieve a concentration in the cell culture medium of between 20 μg/mL and 40 μg/mL.

In some embodiments, in addition to one or more TNFRSF agonists, the cell culture medium further comprises IL-2 at an initial concentration of about 3000 IU/mL and OKT-3 antibody at an initial concentration of about 30 ng/mL, and wherein the one or more TNFRSF agonists comprises a 4-1BB agonist.

In some embodiments the antigen-presenting feeder cells (APCs) are PBMCs. In some embodiments, the ratio of TILs to PBMCs and/or antigen-presenting cells in the rapid expansion and/or the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In some embodiments, the ratio of TILs to PBMCs in the rapid expansion and/or the second expansion is between 1 to 50 and 1 to 300. In some embodiments, the ratio of TILs to PBMCs in the rapid expansion and/or the second expansion is between 1 to 100 and 1 to 200.

In some embodiments, REP and/or the second expansion is performed in flasks with the bulk TILs being mixed with a 100- or 200-fold excess of inactivated feeder cells, 30 mg/mL OKT3 anti-CD3 antibody and 3000 IU/mL IL-2 in 150 ml media. Media replacement is done (generally ⅔ media replacement via respiration with fresh media) until the cells are transferred to an alternative growth chamber. Alternative growth chambers include G-REX flasks and gas permeable containers as more fully discussed below.

In some embodiments, the second expansion (which can include processes referred to as the REP process) is shortened to 7-14 days, as discussed in the examples and figures, wherein the TILs expanded by such a second expansion have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the second expansion is shortened to 11 days.

In some embodiments, REP and/or the second expansion may be performed using T-175 flasks and gas permeable bags as previously described (Tran, et al., J Immunother. 2008, 31, 742-51; Dudley, et al., J Immunother. 2003, 26, 332-42) or gas permeable cultureware (G-Rex flasks), wherein the TILs expanded by such a second expansion have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the second expansion (including expansions referred to as rapid expansions) is performed in T-175 flasks, and about 1×10⁶ TILs suspended in 150 mL of media may be added to each T-175 flask. The TILs may be cultured in a 1 to 1 mixture of CM and AIM-V medium, supplemented with 3000 IU per mL of IL-2 and 30 ng per ml of anti-CD3. The T-175 flasks may be incubated at 37° C. in 5% CO₂, wherein the TILs expanded by such a second expansion have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. Half the media may be exchanged on day 5 using 50/50 medium with 3000 IU per mL of IL-2. In some embodiments, on day 7 cells from two T-175 flasks may be combined in a 3 L bag and 300 mL of AIM V with 5% human AB serum and 3000 IU per mL of IL-2 was added to the 300 ml of TIL suspension. The number of cells in each bag was counted every day or two and fresh media was added to keep the cell count between 0.5 and 2.0×10⁶ cells/mL.

In some embodiments, the second expansion (which can include expansions referred to as REP, as well as those referred to in Step D of FIG. 7) may be performed in 500 mL capacity gas permeable flasks with 100 cm gas-permeable silicon bottoms (G-Rex 100, commercially available from Wilson Wolf Manufacturing Corporation, New Brighton, MN, USA), 5×10⁶ or 10×10⁶ TIL may be cultured with PBMCs in 400 mL of 50/50 medium, supplemented with 5% human AB serum, 3000 IU per mL of IL-2 and 30 ng per ml of anti-CD3 (OKT3), wherein the TILs expanded by such a second expansion have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. The G-Rex 100 flasks may be incubated at 37° C. in 5% CO₂. On day 5, 250 mL of supernatant may be removed and placed into centrifuge bottles and centrifuged at 1500 rpm (491×g) for 10 minutes. The TIL pellets may be re-suspended with 150 mL of fresh medium with 5% human AB serum, 3000 IU per mL of IL-2, and added back to the original G-Rex 100 flasks. When TIL are expanded serially in G-Rex 100 flasks, on day 7 the TIL in each G-Rex 100 may be suspended in the 300 mL of media present in each flask and the cell suspension may be divided into 3 100 mL aliquots that may be used to seed 3 G-Rex 100 flasks. Then 150 mL of AIM-V with 5% human AB serum and 3000 IU per mL of IL-2 may be added to each flask. The G-Rex 100 flasks may be incubated at 37° C. in 5% CO₂ and after 4 days 150 mL of AIM-V with 3000 IU per mL of IL-2 may be added to each G-REX 100 flask. The cells may be harvested on day 14 of culture.

In some embodiments, the second expansion (including expansions referred to as REP) is performed in flasks with the bulk TILs being mixed with a 100- or 200-fold excess of inactivated feeder cells, 30 mg/mL OKT3 anti-CD3 antibody and 3000 IU/mL IL-2 in 150 ml media. In some embodiments, media replacement is done until the cells are transferred to an alternative growth chamber, wherein the TILs expanded by such a second expansion have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, ⅔ of the media is replaced by aspiration of spent media followed by infusion with fresh media. In some embodiments, alternative growth chambers include G-REX flasks and gas permeable containers as more fully discussed below.

In some embodiments, the second expansion culture medium (e.g., sometimes referred to as CM2 or the second cell culture medium), comprises IL-2, OKT-3, as well as the antigen-presenting feeder cells (APCs), as discussed in more detail below.

In some embodiments, the second expansion, for example, Step D according to FIG. 7, is performed in a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100. In some embodiments, the closed system bioreactor is a single bioreactor.

### 1. Feeder Cells and Antigen Presenting Cells

In some embodiments, the second expansion procedures described herein (for example including expansion such as those described in Step D from FIG. 7, as well as those referred to as REP) require an excess of feeder cells during REP TIL expansion and/or during the second expansion, wherein the TILs expanded by such a second expansion procedure have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In many embodiments, the feeder cells are peripheral blood mononuclear cells (PBMCs) obtained from standard whole blood units from healthy blood donors. The PBMCs are obtained using standard methods such as Ficoll-Paque gradient separation.

In general, the allogenic PBMCs are inactivated, either via irradiation or heat treatment, and used in the REP procedures, as described in the examples, which provides an exemplary protocol for evaluating the replication incompetence of irradiate allogeneic PBMCs.

In some embodiments, PBMCs are considered replication incompetent and accepted for use in the TIL expansion procedures described herein if the total number of viable cells on day 14 is less than the initial viable cell number put into culture on day 0 of the REP and/or day 0 of the second expansion (i.e., the start day of the second expansion).

In some embodiments, PBMCs are considered replication incompetent and accepted for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 30 ng/ml OKT3 antibody and 3000 IU/ml IL-2.

In some embodiments, PBMCs are considered replication incompetent and accepted for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 5-60 ng/ml OKT3 antibody and 1000-6000 IU/ml IL-2. In some embodiments, the PBMCs are cultured in the presence of 10-50 ng/ml OKT3 antibody and 2000-5000 IU/ml IL-2. In some embodiments, the PBMCs are cultured in the presence of 20-40 ng/ml OKT3 antibody and 2000-4000 IU/ml IL-2. In some embodiments, the PBMCs are cultured in the presence of 25-35 ng/ml OKT3 antibody and 2500-3500 IU/ml IL-2.

In some embodiments, the antigen-presenting feeder cells are PBMCs. In some embodiments, the antigen-presenting feeder cells are artificial antigen-presenting feeder cells. In some embodiments, the ratio of TILs to antigen-presenting feeder cells in the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In some embodiments, the ratio of TILs to antigen-presenting feeder cells in the second expansion is between 1 to 50 and 1 to 300. In some embodiments, the ratio of TILs to antigen-presenting feeder cells in the second expansion is between 1 to 100 and 1 to 200.

In some embodiments, the second expansion procedures described herein require a ratio of about 2.5×10⁹ feeder cells to about 100×10⁶ TILs. In other embodiments, the second expansion procedures described herein require a ratio of about 2.5×10⁹ feeder cells to about 50×10⁶ TILs. In yet other embodiments, the second expansion procedures described herein require about 2.5×10⁹ feeder cells to about 25×10⁶ TILs.

In some embodiments, the second expansion procedures described herein require an excess of feeder cells during the second expansion. In many embodiments, the feeder cells are peripheral blood mononuclear cells (PBMCs) obtained from standard whole blood units from healthy blood donors. The PBMCs are obtained using standard methods such as Ficoll-Paque gradient separation. In some embodiments, artificial antigen-presenting (aAPC) cells are used in place of PBMCs.

In general, the allogenic PBMCs are inactivated, either via irradiation or heat treatment, and used in the TIL expansion procedures described herein, including the exemplary procedures described in the figures and examples.

In some embodiments, artificial antigen presenting cells are used in the second expansion as a replacement for, or in combination with, PBMCs.

### H. STEP E: Harvest TILS

After the second expansion step, cells can be harvested. In some embodiments the TILs are harvested after one, two, three, four or more expansion steps, for example as provided in FIG. 7. In some embodiments the TILs are harvested after two expansion steps, for example as provided in FIG. 7, wherein the TILs expanded by such expansion steps have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1.

TILs can be harvested in any appropriate and sterile manner, including for example by centrifugation. Methods for TIL harvesting are well known in the art and any such know methods can be employed with the present process. In some embodiments, TILs are harvest using an automated system.

Cell harvesters and/or cell processing systems are commercially available from a variety of sources, including, for example, Fresenius Kabi, Tomtec Life Science, Perkin Elmer, and Inotech Biosystems International, Inc. Any cell based harvester can be employed with the present methods. In some embodiments, the cell harvester and/or cell processing systems is a membrane-based cell harvester. In some embodiments, cell harvesting is via a cell processing system, such as the LOVO system (manufactured by Fresenius Kabi). The term “LOVO cell processing system” also refers to any instrument or device manufactured by any vendor that can pump a solution comprising cells through a membrane or filter such as a spinning membrane or spinning filter in a sterile and/or closed system environment, allowing for continuous flow and cell processing to remove supernatant or cell culture media without pelletization. In some embodiments, the cell harvester and/or cell processing system can perform cell separation, washing, fluid-exchange, concentration, and/or other cell processing steps in a closed, sterile system.

In some embodiments, the harvest, for example, Step E according to FIG. 7, is performed from a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100. In some embodiments, the closed system bioreactor is a single bioreactor. In some embodiments, the closed system is accessed via syringes under sterile conditions in order to maintain the sterility and closed nature of the system.

### L. STEP F: Final Formulation/Transfer to Infusion Bag

After Steps A through E as provided in an exemplary order in FIG. 7 and as outlined in detailed above and herein are complete, genetically modified TILs are transferred to a container for use in administration to a patient, wherein the genetically modified TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, once a therapeutically sufficient number of genetically modified TILs are obtained using the expansion methods described above, they are transferred to a container for use in administration to a patient, wherein the genetically modified TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1.

In some embodiments, TILs expanded using APCs of the present disclosure are administered to a patient as a pharmaceutical composition, wherein the expanded TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the pharmaceutical composition is a suspension of genetically modified TILs in a sterile buffer. TILs expanded using the methods of the present disclosure may be administered by any suitable route as known in the art, wherein the such TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. In some embodiments, the TILs are administered as a single intra-arterial or intravenous infusion, which preferably lasts approximately 30 to 60 minutes, wherein such TILs have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1. Other suitable routes of administration include intraperitoneal, intrathecal, and intralymphatic.

### IV. Pharmaceutical Compositions, Dosages, and Dosing Regimens

In some embodiments, TILs that have been genetically modified via TALEN gene-editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1, and expanded using the methods of the present disclosure (referred to herein as “CISH^{lo} or CISH^{lo}/PD-1l° TILs”), are administered to a patient as a pharmaceutical composition. In some embodiments, the pharmaceutical composition is a suspension of CISH^{lo} or CISH^{lo}/PD-1^{lo} TILs in a sterile buffer. In some embodiments, CISH^{lo} or CISH^{lo}/PD-1^{lo} TILs expanded using PBMCs of the present disclosure may be administered by any suitable route as known in the art. In some embodiments, CISH^{lo} or CISH^{lo}/PD-1^{lo} TILs are administered as a single intra-arterial or intravenous infusion, which preferably lasts approximately 30 to 60 minutes. Other suitable routes of administration include intraperitoneal, intrathecal, and intralymphatic administration.

Any suitable dose of CISH^{lo} or CISH^{lo}/PD-1^{lo} TILs can be administered. In some embodiments, from about 2.3×10¹⁰ to about 13.7×10¹⁰ CISH^{lo} or CISH^{lo}/PD-1^{lo} TILs are administered, with an average of around 7.8×10¹⁰ CISH^{lo}/PD-1^{lo} TILs, particularly if the cancer is melanoma. In some embodiments, about 1.2×10¹⁰ to about 4.3×10¹⁰ of CISH^{lo} or CISH^{lo}/PD-1^{lo} TILs are administered. In some embodiments, about 3×10¹⁰ to about 12×10¹⁰ CISH^{lo} or CISH^{lo}/PD-1^{lo} TILs are administered. In some embodiments, about 4×10¹⁰ to about 10×10¹⁰ CISH^{lo} or CISH^{lo}/PD-1^{lo} TILs are administered. In some embodiments, about 5×10¹⁰ to about 8×10¹⁰ CISH^{lo} or CISH^{lo}/PD-1^{lo} TILs are administered. In some embodiments, about 6×10¹⁰ to about 8×10¹⁰ CISH^{lo} or CISH^{lo}/PD-1^{lo} TILs are administered. In some embodiments, about 7×10¹⁰ to about 8×10¹⁰ CISH^{lo} or CISH^{lo}/PD-1^{lo} TILs are administered.

In some embodiments, the therapeutically effective dosage is about 2.3×10¹⁰ to about 13.7×10¹⁰. In some embodiments, the therapeutically effective dosage is about 7.8×10¹⁰ CISH^{lo} or CISH^{lo}/PD-1^{lo} TILs, particularly if the cancer is melanoma. In some embodiments, the therapeutically effective dosage is about 1.2×10¹⁰ to about 4.3×10¹⁰ of CISH^{lo} or CISH^{lo}/PD-1^{lo} TILs. In some embodiments, the therapeutically effective dosage is about 3×10¹⁰ to about 12×10¹⁰ CISH^{lo}/PD-1^{lo} TILs. In some embodiments, the therapeutically effective dosage is about 4×10¹⁰ to about 10×10¹⁰ CISH^{lo} or CISH^{lo}/PD-1^{lo} TILs. In some embodiments, the therapeutically effective dosage is about 5×10¹⁰ to about 8×10¹⁰ CISH^{lo} or CISH^{lo}/PD-1^{lo} TILs. In some embodiments, the therapeutically effective dosage is about 6×10¹⁰ to about 8×10¹⁰ CISH^{lo} or CISH^{lo}/PD-1^{lo} TILs. In some embodiments, the therapeutically effective dosage is about 7×10¹⁰ to about 8×10¹⁰ CISH^{lo} or CISH^{lo}/PD-1l° TILs.

In some embodiments, the number of the CISH^{lo} or CISH^{lo}/PD-1^{lo} TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, the number of the CISH^{lo} or CISH^{lo}/PD-1^{lo} TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, the concentration of the CISH^{lo} or CISH^{lo}/PD-1^{lo} TILs provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of the CISH^{lo} or CISH^{lo}/PD-1^{lo} TILs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of the CISH^{lo} or CISH^{lo}/PD-1^{lo} TILs provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the CISH^{lo} or CISH^{lo}/PD-1^{lo} TILs provided in the pharmaceutical compositions of the invention is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of the CISH^{lo} or CISH^{lo}/PD-1^{lo} TILs provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the CISH^{lo} or CISH^{lo}/PD-1^{lo} TILs provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

The CISH^{lo} or CISH^{lo}/PD-1^{lo} TILs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the CISH^{lo} or CISH^{lo}/PD-1^{lo} TILs may also be used if appropriate. The amounts of the pharmaceutical compositions administered using the methods herein, such as the dosages of CISH^{lo} or CISH^{lo}/PD-1^{lo} TILs, will be dependent on the human or mammal being treated, the severity of the disorder or condition, the rate of administration, the disposition of the active pharmaceutical ingredients and the discretion of the prescribing physician.

In some embodiments, CISH^{lo} or CISH^{lo}/PD-1^{lo} TILs may be administered in a single dose. Such administration may be by injection, e.g., intravenous injection. In some embodiments, CISH^{lo} or CISH^{lo}/PD-1^{lo} TILs may be administered in multiple doses. Dosing may be once, twice, three times, four times, five times, six times, or more than six times per year. Dosing may be once a month, once every two weeks, once a week, or once every other day. Administration of TILs may continue as long as necessary.

In some embodiments, an effective dosage of CISH^{lo} or CISH^{lo}/PD-1^{lo} TILs is about 1×10⁶, 2×10⁶, 3×10⁶, 4×10⁶ 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, an effective dosage of CISH^{lo} or CISH^{lo}/PD-1^{lo} TILs is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, an effective dosage of CISH^{lo} or CISH^{lo}/PD-1^{lo} TILs is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of CISH^{lo} or CISH^{lo}/PD-1^{lo} TILs is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 10⁵ mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg.

An effective amount of the CISH^{lo} or CISH^{lo}/PD-1^{lo} TILs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation, or by inhalation.

In other embodiments, the invention provides an infusion bag comprising the therapeutic population of CISH^{lo} or CISH^{lo}/PD-1^{lo} TILs described in any of the preceding paragraphs above.

In other embodiments, the invention provides a tumor infiltrating lymphocyte (TIL) composition comprising the therapeutic population of CISH^{lo} or CISH^{lo}/PD-1^{lo} TILs described in any of the preceding paragraphs above and a pharmaceutically acceptable carrier.

In other embodiments, the invention provides an infusion bag comprising the CISH® or CISH^{lo}/PD-1^{lo} TIL composition described in any of the preceding paragraphs above.

In other embodiments, the invention provides a cryopreserved preparation of the therapeutic population of CISH^{lo} or CISH^{lo}/PD-1^{lo} TILs described in any of the preceding paragraphs above.

In other embodiments, the invention provides a tumor infiltrating lymphocyte (TIL) composition comprising the therapeutic population of CISH^{lo} or CISH^{lo}/PD-1^{lo} TILs described in any of the preceding paragraphs above and a cryopreservation media.

In other embodiments, the invention provides the CISH^{lo} or CISH^{lo}/PD-1^{lo} TIL composition described in any of the preceding paragraphs above modified such that the cryopreservation media contains DMSO.

In other embodiments, the invention provides the CISH^{lo} or CISH^{lo}/PD-1^{lo} TIL composition described in any of the preceding paragraphs above modified such that the cryopreservation media contains 7-10% DMSO.

In other embodiments, the invention provides a cryopreserved preparation of the CISH^{lo} or CISH^{lo}/PD-1^{lo} TIL composition described in any of the preceding paragraphs above.

In some embodiments, CISH^{lo} or CISH^{lo}/PD-1^{lo} TILs expanded using the methods of the present disclosure are administered to a patient as a pharmaceutical composition. In some embodiments, the pharmaceutical composition is a suspension of CISH^{lo} or CISH^{lo}/PD-1^{lo} TILs in a sterile buffer. CISH^{lo} or CISH^{lo}/PD-1^{lo} TILs expanded using PBMCs of the present disclosure may be administered by any suitable route as known in the art. In some embodiments, the CISH^{lo} or CISH^{lo}/PD-1^{lo} TILs are administered as a single intra-arterial or intravenous infusion, which preferably lasts approximately 30 to 60 minutes. Other suitable routes of administration include intraperitoneal, intrathecal, and intralymphatic administration.

Any suitable dose of CISH^{lo} or CISH^{lo}/PD-1^{lo} TILs can be administered. In some embodiments, from about 2.3×10¹⁰ to about 13.7×10¹⁰ CISH^{lo} or CISH^{lo}/PD-1^{lo} TILs are administered, with an average of around 7.8×10¹⁰ CISH^{lo} or CISH^{lo}/PD-1^{lo} TILs, particularly if the cancer is melanoma. In some embodiments, about 1.2×10¹⁰ to about 4.3×10¹⁰ of CISH^{lo} or CISH^{lo}/PD-1^{lo} TILs are administered. In some embodiments, about 3×10¹⁰ to about 12×10¹⁰ CISH^{lo} or CISH^{lo}/PD-1^{lo} TILs are administered. In some embodiments, about 4×10¹⁰ to about 10×10¹⁰ CISH^{lo} or CISH^{lo}/PD-1^{lo} TILs are administered. In some embodiments, about 5×10¹⁰ to about 8×10¹⁰ CISH^{lo} or CISH^{lo}/PD-1^{lo} TILs are administered. In some embodiments, about 6×10¹⁰ to about 8×10¹⁰ CISH^{lo} or CISH^{lo}/PD-1^{lo} TILs are administered.

In some embodiments, about 7×10¹⁰ to about 8×10¹⁰ CISH^{lo} or CISH^{lo}/PD-1^{lo} TILs are administered. In some embodiments, therapeutically effective dosage is about 2.3×10¹⁰ to about 13.7×10¹⁰. In some embodiments, therapeutically effective dosage is about 7.8×10¹⁰ CISH^{lo} or CISH^{lo}/PD-1^{lo} TILs, particularly of the cancer is melanoma. In some embodiments, therapeutically effective dosage is about 1.2×10¹⁰ to about 4.3×10¹⁰ of CISH^{lo} or CISH^{lo}/PD-1^{lo} TILs. In some embodiments, therapeutically effective dosage is about 3×10¹⁰ to about 12×10¹⁰ CISH^{lo} or CISH^{lo}/PD-1^{lo} TILs. In some embodiments, therapeutically effective dosage is about 4×10¹⁰ to about 10×10¹⁰ CISH^{lo} or CISH^{lo}/PD-1^{lo} TILs. In some embodiments, therapeutically effective dosage is about 5×10¹⁰ to about 8×10¹⁰ CISH^{lo} or CISH^{lo}/PD-1^{lo} TILs. In some embodiments, therapeutically effective dosage is about 6×10¹⁰ to about 8×10¹⁰ CISH^{lo} or CISH^{lo}/PD-1^{lo} TILs. In some embodiments, therapeutically effective dosage is about 7×10¹⁰ to about 8×10¹⁰ CISH^{lo} or CISH^{lo}/PD-1^{lo} TILs.

In some embodiments, the number of the CISH^{lo} or CISH^{lo}/PD-1^{lo} TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹, 2×10¹, 3×10¹, 4×10¹, 5×10¹, 6×10¹, 7×10¹, 8×10¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, the number of the CISH^{lo} or CISH^{lo}/PD-1^{lo} TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, the concentration of the CISH^{lo} or CISH^{lo}/PD-1^{lo} TILs provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of the CISH^{lo} or CISH^{lo}/PD-1^{lo} TILs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of the CISH^{lo} or CISH^{lo}/PD-1^{lo} TILs provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the CISH^{lo} or CISH^{lo}/PD-1^{lo} TILs provided in the pharmaceutical compositions of the invention is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of the CISH^{lo} or CISH^{lo}/PD-1^{lo} TILs provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the CISH^{lo} or CISH^{lo}/PD-1^{lo} TILs provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

The CISH^{lo} or CISH^{lo}/PD-1^{lo} TILs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the CISH^{lo} or CISH^{lo}/PD-1^{lo} TILs may also be used if appropriate. The amounts of the pharmaceutical compositions administered using the methods herein, such as the dosages of CISH^{lo} or CISH^{lo}/PD-1^{lo} TILs, will be dependent on the human or mammal being treated, the severity of the disorder or condition, the rate of administration, the disposition of the active pharmaceutical ingredients and the discretion of the prescribing physician.

In some embodiments, CISH^{lo} or CISH^{lo}/PD-1^{lo} TILs may be administered in a single dose. Such administration may be by injection, e.g., intravenous injection. In some embodiments, CISH^{lo} or CISH^{lo}/PD-1^{lo} TILs may be administered in multiple doses. Dosing may be once, twice, three times, four times, five times, six times, or more than six times per year. Dosing may be once a month, once every two weeks, once a week, or once every other day. Administration of CISH^{lo} or CISH^{lo}/PD-1^{lo} TILs may continue as long as necessary.

In some embodiments, an effective dosage of CISH^{lo} or CISH^{lo}/PD-1^{lo} TILs is about 1×10⁶, 2×10⁶, 3×10⁶, 4×10⁶ 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, an effective dosage of TILs is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10² to 1×10¹³.

In some embodiments, an effective dosage of CISH^{lo} or CISH^{lo}/PD-1^{lo} TILs is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of CISH^{lo} or CISH^{lo}/PD-1^{lo} TILs is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 10⁵ mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg.

An effective amount of the CISH^{lo} or CISH^{lo}/PD-1^{lo} TILs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation, or by inhalation.

### V. Methods of Treating Patients

Methods of treatment begin with the initial TIL collection and culture of TILs. Such methods have been both described in the art by, for example, Jin et al., J. Immunotherapy, 2012, 35(3):283-292, incorporated by reference herein in its entirety. Embodiments of methods of treatment are described throughout the sections below, including the Examples.

The expanded CISH^{lo} or CISH^{lo}/PD-1^{lo} TILs of the invention can be expanded in accordance with any embodiment of the methods as described in FIG. 7 herein or as described in PCT/US2017/058610, PCT/US2018/012605, or PCT/US2018/012633, find use in the treatment of patients with cancer (for example, as described in Goff, et al., J. Clinical Oncology, 2016, 34(20):2389-239, as well as the supplemental content; incorporated by reference herein in its entirety. In some embodiments, TIL are grown from resected deposits of metastatic melanoma as previously described (see, Dudley, et al., J Immunother., 2003, 26:332-342; incorporated by reference herein in its entirety).

Cell phenotypes of cryopreserved samples of infusion bag CISH^{lo} or CISH^{lo}/PD-1l° TIL can be analyzed by flow cytometry (e.g., FlowJo) for surface markers CD3, CD4, CD8, CCR7, and CD45RA (BD BioSciences), as well as by any of the methods described herein. Serum cytokines were measured by using standard enzyme-linked immunosorbent assay techniques. A rise in serum IFN-γ can be defined as >100 μg/mL.

Measures of efficacy can include the disease control rate (DCR) as well as overall response rate (ORR), as known in the art as well as described herein.

### A. Methods of Treating Cancers and Other Diseases

The compositions and methods described herein can be used in a method for treating diseases. In some embodiments, they are for use in treating hyperproliferative disorders. They may also be used in treating other disorders as described herein and in the following paragraphs.

In some embodiments, the hyperproliferative disorder is cancer. In some embodiments, the hyperproliferative disorder is a solid tumor cancer. In some embodiments, the solid tumor cancer is selected from the group consisting of glioblastoma (GBM), gastrointestinal cancer, melanoma, ovarian cancer, endometrial cancer, thyroid cancer, colorectal cancer, cervical cancer, non-small-cell lung cancer (NSCLC), lung cancer, bladder cancer, breast cancer, triple negative breast cancer, cancer caused by human papilloma virus, head and neck cancer (including head and neck squamous cell carcinoma (HNSCC)), renal cancer, and renal cell carcinoma. In some embodiments, the hyperproliferative disorder is a hematological malignancy. In some embodiments, the solid tumor cancer is selected from the group consisting of chronic lymphocytic leukemia, acute lymphoblastic leukemia, diffuse large B cell lymphoma, non-Hodgkin's lymphoma, Hodgkin's lymphoma, follicular lymphoma, and mantle cell lymphoma.

In some embodiments, the cancer is a hypermutated cancer phenotype. Hypermutated cancers are extensively described in Campbell, et al. (Cell, 171:1042-1056 (2017); incorporated by reference herein in its entirety for all purposes). In some embodiments, a hypermutated tumors comprise between 9 and 10 mutations per megabase (Mb). In some embodiments, pediatric hypermutated tumors comprise 9.91 mutations per megabase (Mb). In some embodiments, adult hypermutated tumors comprise 9 mutations per megabase (Mb). In some embodiments, enhanced hypermutated tumors comprise between 10 and 100 mutations per megabase (Mb). In some embodiments, enhanced pediatric hypermutated tumors comprise between 10 and 100 mutations per megabase (Mb). In some embodiments, enhanced adult hypermutated tumors comprise between 10 and 100 mutations per megabase (Mb). In some embodiments, an ultra-hypermutated tumors comprise greater than 100 mutations per megabase (Mb). In some embodiments, pediatric ultra-hypermutated tumors comprise greater than 100 mutations per megabase (Mb). In some embodiments, adult ultra-hypermutated tumors comprise greater than 100 mutations per megabase (Mb).

In some embodiments, the hypermutated tumors have mutations in replication repair pathways. In some embodiments, the hypermutated tumors have mutations in replication repair associated DNA polymerases. In some embodiments, the hypermutated tumors have microsatellite instability. In some embodiments, the ultra-hypermutated tumors have mutations in replication repair associated DNA polymerases and have microsatellite instability. In some embodiments, hypermutation in the tumor is correlated with response to immune checkpoint inhibitors. In some embodiments, hypermutated tumors are resistant to treatment with immune checkpoint inhibitors. In some embodiments, hypermutated tumors can be treated using the TILs of the present invention. In some embodiments, hypermutation in the tumor is caused by environmental factors (extrinsic exposures). For example, UV light can be the primary cause of the high numbers of mutations in malignant melanoma (see, for example, Pfeifer, G. P., You, Y. H., and Besaratinia, A. (2005). Mutat. Res. 571, 19-31.; Sage, E. (1993). Photochem. Photobiol. 57, 163-174.). In some embodiments, hypermutation in the tumor can be caused by the greater than 60 carcinogens in tobacco smoke for tumors of the lung and larynx, as well as other tumors, due to direct mutagen exposure (see, for example, Pleasance, E. D., Stephens, P. J., O'Meara, S., McBride, D. J., Meynert, A., Jones, D., Lin, M. L., Beare, D., Lau, K. W., Greenman, C., et al. (2010). Nature 463, 184-190). In some embodiments, hypermutation in the tumor is caused by dysregulation of apolipoprotein B mRNA editing enzyme, catalytic polypeptide-like (APOBEC) family members, which has been shown to result in increased levels of C to T transitions in a wide range of cancers (see, for example, Roberts, S. A., Lawrence, M. S., Klimczak, L. J., Grimm, S. A., Fargo, D., Stojanov, P., Kiezun, A., Kryukov, G. V., Carter, S. L., Saksena, G., et al. (2013). Nat. Genet. 45, 970-976). In some embodiments, hypermutation in the tumor is caused by defective DNA replication repair by mutations that compromise proofreading, which is performed by the major replicative enzymes Pol3 and Pold1. In some embodiments, hypermutation in the tumor is caused by defects in DNA mismatch repair, which are associated with hypermutation in colorectal, endometrial, and other cancers (see, for example, Kandoth, C., Schultz, N., Chemiack, A. D., Akbani, R., Liu, Y., Shen, H., Robertson, A. G., Pashtan, I., Shen, R., Benz, C. C., et al.; (2013). Nature 497, 67-73.; Muzny, D. M., Bainbridge, M. N., Chang, K., Dinh, H.H., Drummond, J. A., Fowler, G., Kovar, C. L., Lewis, L. R., Morgan, M. B., Newsham, I. F., et al.; (2012). Nature 487, 330-337). In some embodiments, DNA replication repair mutations are also found in cancer predisposition syndromes, such as constitutional or biallelic mismatch repair deficiency (CMMRD), Lynch syndrome, and polymerase proofreading-associated polyposis (PPAP).

In some embodiments, the invention includes a method of treating a cancer with a population of CISH^{lo} or CISH^{lo}/PD-1^{lo} TILs, wherein the cancer is a hypermutated cancer. In some embodiments, the invention includes a method of treating a cancer with a population of CISH^{lo} or CISH^{lo}/PD-1^{lo} TILs, wherein the cancer is an enhanced hypermutated cancer. In some embodiments, the invention includes a method of treating a cancer with a population of CISH^{lo} or CISH^{lo}/PD-1^{lo} TILs, wherein the cancer is an ultra-hypermutated cancer.

In some embodiments, the invention includes a method of treating a cancer with a population of CISH^{lo} or CISH^{lo}/PD-1^{lo} TILs, wherein a patient is pre-treated with non-myeloablative chemotherapy prior to an infusion of CISH^{lo} or CISH^{lo}/PD-1^{lo} TILs according to the present disclosure. In some embodiments, the non-myeloablative chemotherapy is cyclophosphamide 60 mg/kg/d for 2 days (days 27 and 26 prior to CISH^{lo} or CISH^{lo}/PD-1l° TIL infusion) and fludarabine 25 mg/m²/d for 5 days (days 27 to 23 prior to CISH^{lo}/PD-1^{lo} TIL infusion). In some embodiments, the non-myeloablative chemotherapy is cyclophosphamide 60 mg/kg/d for 2 days (days 27 and 26 prior to CISH^{lo} or CISH^{lo}/PD-1l° TIL infusion) and fludarabine 25 mg/m2/d for 3 days (days 27 to 25 prior to CISH^{lo} or CISH^{lo}/PD-1^{lo} TIL infusion). In some embodiments, the non-myeloablative chemotherapy is cyclophosphamide 60 mg/kg/d for 2 days (days 27 and 26 prior to CISH^{lo} or CISH^{lo}/PD-1l° TIL infusion) followed by fludarabine 25 mg/m2/d for 3 days (days 25 to 23 prior to CISH^{lo} or CISH^{lo}/PD-1^{lo} TIL infusion). In some embodiments, after non-myeloablative chemotherapy and CISH^{lo} or CISH^{lo}/PD-1^{lo} TIL infusion (at day 0) according to the present disclosure, the patient receives an intravenous infusion of IL-2 intravenously at 720,000 IU/kg every 8 hours to physiologic tolerance.

### 1. Optional Lymphodepletion Preconditioning of Patients

In some embodiments, the invention includes a method of treating a cancer with a population of genetically modified TILs that have been genetically modified via TALEN gene editing by introducing into the TILs nucleic acids, such as mRNAs, encoding one or more TALE-nucleases to selectively inactivate by DNA cleavage a gene encoding CISH, wherein the one or more TALE-nucleases comprise a TALE-nuclease that is directed against the nucleic acid sequence of SEQ ID NO: 175 as a CISH gene target sequence, and optionally by introducing into the TILs nucleic acids, such as mRNAs, encoding one or more TALE-nucleases to selectively inactivate by DNA cleavage a gene encoding PD-1, wherein a patient is pre-treated with non-myeloablative chemotherapy prior to an infusion of such TILs according to the present disclosure. In some embodiments, the invention includes a population of CISH^{lo} or CISH^{lo}/PD-1^{lo} TILs for use in the treatment of cancer in a patient which has been pre-treated with non-myeloablative chemotherapy. In some embodiments, the population of CISH^{lo} or CISH^{lo}/PD-1^{lo} TILs is for administration by infusion. In some embodiments, the non-myeloablative chemotherapy is cyclophosphamide 60 mg/kg/d for 2 days (days 27 and 26 prior to TIL infusion) and fludarabine 25 mg/m²/d for 5 days (days 27 to 23 prior to TIL infusion). In some embodiments, the non-myeloablative chemotherapy is cyclophosphamide 60 mg/kg/d for 2 days (days 27 and 26 prior to CISH^{lo} or CISH^{lo}/PD-1^{lo} TIL infusion) and fludarabine 25 mg/m2/d for 3 days (days 27 to 25 prior to CISH^{lo} or CISH^{lo}/PD-1^{lo} TIL infusion). In some embodiments, the non-myeloablative chemotherapy is cyclophosphamide 60 mg/kg/d for 2 days (days 27 and 26 prior to CISH^{lo} or CISH^{lo}/PD-1^{lo} TIL infusion) followed by fludarabine 25 mg/m2/d for 3 days (days 25 to 23 prior to CISH^{lo} or CISH^{lo}/PD-1^{lo} TIL infusion). In some embodiments, after non-myeloablative chemotherapy and CISH^{lo} or CISH^{lo}/PD-1^{lo} TIL infusion (at day 0) according to the present disclosure, the patient receives an intravenous infusion of IL-2 (aldesleukin, commercially available as PROLEUKIN) intravenously at 720,000 IU/kg every 8 hours to physiologic tolerance. In certain embodiments, the population of CISH^{lo} or CISH^{lo}/PD-1^{lo} TILs is for use in treating cancer in combination with IL-2, wherein the IL-2 is administered after the population of such TILs.

Experimental findings indicate that lymphodepletion prior to adoptive transfer of tumor-specific T lymphocytes plays a key role in enhancing treatment efficacy by eliminating regulatory T cells and competing elements of the immune system (‘cytokine sinks’). Accordingly, some embodiments of the invention utilize a lymphodepletion step (sometimes also referred to as “immunosuppressive conditioning”) on the patient prior to the introduction of the TILs of the invention.

In general, lymphodepletion is achieved using administration of fludarabine or cyclophosphamide (the active form being referred to as mafosfamide) and combinations thereof. Such methods are described in Gassner, et al., Cancer Immunol. Immunother. 2011, 60, 75-85, Muranski, et al., Nat. Clin. Pract. Oncol., 2006, 3, 668-681, Dudley, et al., J. Clin. Oncol., 2008, 26, 5233-5239, and Dudley, et al., J. Clin. Oncol., 2005, 23, 2346-2357, all of which are incorporated by reference herein in their entireties.

In some embodiments, the fludarabine is administered at a concentration of 0.5 μg/mL-10 μg/mL fludarabine. In some embodiments, the fludarabine is administered at a concentration of 1 μg/mL fludarabine. In some embodiments, the fludarabine treatment is administered for 1 day, 2 days, 3 days, 4 days, 5 days, 6 days, or 7 days or more. In some embodiments, the fludarabine is administered at a dosage of 10 mg/kg/day, 15 mg/kg/day, 20 mg/kg/day, 25 mg/kg/day, 30 mg/kg/day, 35 mg/kg/day, 40 mg/kg/day, or 45 mg/kg/day. In some embodiments, the fludarabine treatment is administered for 2-7 days at 35 mg/kg/day. In some embodiments, the fludarabine treatment is administered for 4-5 days at 35 mg/kg/day. In some embodiments, the fludarabine treatment is administered for 4-5 days at 25 mg/kg/day.

In some embodiments, the mafosfamide, the active form of cyclophosphamide, is obtained at a concentration of 0.5 g/mL-10 μg/mL by administration of cyclophosphamide. In some embodiments, mafosfamide, the active form of cyclophosphamide, is obtained at a concentration of 1 μg/mL by administration of cyclophosphamide. In some embodiments, the cyclophosphamide treatment is administered for 1 day, 2 days, 3 days, 4 days, 5 days, 6 days, or 7 days or more. In some embodiments, the cyclophosphamide is administered at a dosage of 100 mg/m²/day, 150 mg/m²/day, 175 mg/m²/day, 200 mg/m²/day, 225 mg/m²/day, 250 mg/m²/day, 275 mg/m²/day, or 300 mg/m²/day. In some embodiments, the cyclophosphamide is administered intravenously (i.e., i.v.) In some embodiments, the cyclophosphamide treatment is administered for 2-7 days at 35 mg/kg/day. In some embodiments, the cyclophosphamide treatment is administered for 4-5 days at 250 mg/m²/day i.v. In some embodiments, the cyclophosphamide treatment is administered for 4 days at 250 mg/m²/day i.v.

In some embodiments, lymphodepletion is performed by administering the fludarabine and the cyclophosphamide together to a patient. In some embodiments, fludarabine is administered at 25 mg/m²/day i.v. and cyclophosphamide is administered at 250 mg/m²/day i.v. over 4 days.

In some embodiments, the lymphodepletion is performed by administration of cyclophosphamide at a dose of 60 mg/m²/day for two days followed by administration of fludarabine at a dose of 25 mg/m²/day for five days.

In some embodiments, the lymphodepletion is performed by administration of cyclophosphamide at a dose of 60 mg/m²/day for two days followed by administration of fludarabine at a dose of 25 mg/m²/day for three days.

In some embodiments, the lymphodepletion is performed by administration of cyclophosphamide at a dose of 60 mg/m²/day for two days and administration of fludarabine at a dose of 25 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of 60 mg/m²/day for two days and administration of fludarabine at a dose of 25 mg/m²/day for three days, wherein cyclophosphamide and fludarabine are both administered on the first two days, and wherein the lymphodepletion is performed in three days in total.

In some embodiments, the lymphodepletion is performed by administration of cyclophosphamide at a dose of about 50 mg/m²/day for two days and administration of fludarabine at a dose of about 25 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 50 mg/m²/day for two days followed by administration of fludarabine at a dose of about 25 mg/m²/day for three days, wherein the lymphodepletion is performed in five days in total.

In some embodiments, the lymphodepletion is performed by administration of cyclophosphamide at a dose of about 50 mg/m²/day for two days and administration of fludarabine at a dose of about 25 mg/m²/day for three days, wherein cyclophosphamide and fludarabine are both administered on the first two days, and wherein the lymphodepletion is performed in three days in total.

In some embodiments, the lymphodepletion is performed by administration of cyclophosphamide at a dose of about 50 mg/m²/day for two days and administration of fludarabine at a dose of about 20 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 50 mg/m²/day for two days followed by administration of fludarabine at a dose of about 20 mg/m²/day for three days, wherein the lymphodepletion is performed in five days in total.

In some embodiments, the lymphodepletion is performed by administration of cyclophosphamide at a dose of about 50 mg/m²/day for two days and administration of fludarabine at a dose of about 20 mg/m²/day for three days, wherein cyclophosphamide and fludarabine are both administered on the first two days, and wherein the lymphodepletion is performed in three days in total.

In some embodiments, the lymphodepletion is performed by administration of cyclophosphamide at a dose of about 40 mg/m²/day for two days and administration of fludarabine at a dose of about 20 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 40 mg/m²/day for two days followed by administration of fludarabine at a dose of about 20 mg/m²/day for three days, wherein the lymphodepletion is performed in five days in total.

In some embodiments, the lymphodepletion is performed by administration of cyclophosphamide at a dose of about 40 mg/m²/day for two days and administration of fludarabine at a dose of about 20 mg/m²/day for three days, wherein cyclophosphamide and fludarabine are both administered on the first two days, and wherein the lymphodepletion is performed in three days in total.

In some embodiments, the lymphodepletion is performed by administration of cyclophosphamide at a dose of about 40 mg/m²/day for two days and administration of fludarabine at a dose of about 15 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 40 mg/m²/day for two days followed by administration of fludarabine at a dose of about 15 mg/m²/day for three days, wherein the lymphodepletion is performed in five days in total.

In some embodiments, the lymphodepletion is performed by administration of cyclophosphamide at a dose of about 40 mg/m²/day for two days and administration of fludarabine at a dose of about 15 mg/m²/day for three days, wherein cyclophosphamide and fludarabine are both administered on the first two days, and wherein the lymphodepletion is performed in three days in total.

In some embodiments, the lymphodepletion is performed by administration of cyclophosphamide at a dose of 60 mg/m²/day and fludarabine at a dose of 25 mg/m²/day for two days followed by administration of fludarabine at a dose of 25 mg/m²/day for three days.

In some embodiments, the lymphodepletion is performed by administration of cyclophosphamide at a dose of 60 mg/m²/day and fludarabine at a dose of 25 mg/m²/day for two days followed by administration of fludarabine at a dose of 25 mg/m²/day for one day.

In some embodiments, the cyclophosphamide is administered with mesna. In some embodiments, mesna is administered at 15 mg/kg. In some embodiments where mesna is infused, and if infused continuously, mesna can be infused over approximately 2 hours with cyclophosphamide (on Days −5 and/or −4), then at a rate of 3 mg/kg/hour for the remaining 22 hours over the 24 hours starting concomitantly with each cyclophosphamide dose.

In some embodiments, the method of the invention further comprises the step of treating the patient with an IL-2 regimen starting on the day after administration of the CISH^{lo}/PD-1^{lo} TILs to the patient.

In some embodiments, the method of the invention further comprises the step of treating the patient with an IL-2 regimen starting on the same day as administration of the CISH^{lo}/PD-1^{lo} TILs to the patient.

In some embodiments, the lymphodepletion comprises 5 days of preconditioning treatment. In some embodiments, the days are indicated as days −5 through −1, or Day 0 through Day 4. In some embodiments, the regimen comprises cyclophosphamide on days −5 and −4 (i.e., days 0 and 1). In some embodiments, the regimen comprises intravenous cyclophosphamide on days −5 and −4 (i.e., days 0 and 1). In some embodiments, the regimen comprises 60 mg/kg intravenous cyclophosphamide on days −5 and −4 (i.e., days 0 and 1). In some embodiments, the cyclophosphamide is administered with mesna. In some embodiments, the regimen further comprises fludarabine. In some embodiments, the regimen further comprises intravenous fludarabine. In some embodiments, the regimen further comprises 25 mg/m² intravenous fludarabine. In some embodiments, the regimen further comprises 25 mg/m² intravenous fludarabine on days −5 through −1 (i.e., days 0 through 4). In some embodiments, the regimen further comprises 25 mg/m² intravenous fludarabine on days −5 through −1 (i.e., days 0 through 4).

In some embodiments, the non-myeloablative lymphodepletion regimen comprises the steps of administration of cyclophosphamide at a dose of 60 mg/m2/day and fludarabine at a dose of 25 mg/m²/day for two days followed by administration of fludarabine at a dose of 25 mg/m²/day for five days.

In some embodiments, the non-myeloablative lymphodepletion regimen comprises the steps of administration of cyclophosphamide at a dose of 60 mg/m2/day and fludarabine at a dose of 25 mg/m²/day for two days followed by administration of fludarabine at a dose of 25 mg/m²/day for three days.

In some embodiments, the non-myeloablative lymphodepletion regimen comprises the steps of administration of cyclophosphamide at a dose of 60 mg/m2/day for two days followed by administration of fludarabine at a dose of 25 mg/m²/day for five days.

In some embodiments, the non-myeloablative lymphodepletion regimen comprises the steps of administration of cyclophosphamide at a dose of 60 mg/m2/day for two days followed by administration of fludarabine at a dose of 25 mg/m²/day for three days.

In some embodiments, the non-myeloablative lymphodepletion regimen comprises the steps of administration of cyclophosphamide at a dose of 60 mg/m2/day and fludarabine at a dose of 25 mg/m²/day for two days followed by administration of fludarabine at a dose of 25 mg/m²/day for one day.

In some embodiments, the non-myeloablative lymphodepletion regimen is administered per the table below:

TABLE 4
Depletion protocol.

| | | | | | | | | | | |
|---|---|---|---|---|---|---|---|---|---|---|
| Treatment Administration | −5 | −4 | −3 | −2 | −1 | 0 | 1 | 2 | 3 | 4 |

| | | | | | | |
|---|---|---|---|---|---|---|
| Cyclophosphamide 60 mg/kg | X | X | | | | |
| Mesna | X | X | | | | |
| Fludarabine 25 mg/m²/day | X | X | X | X | X | |
| TIL-based immunotherapy | | | | | | X |
| infusion | | | | | | |

### 2. IL-2 Regimens

In some embodiments, the IL-2 regimen comprises a high-dose IL-2 regimen, wherein the high-dose IL-2 regimen comprises aldesleukin, or a biosimilar or variant thereof, administered intravenously starting on the day after administering a therapeutically effective portion of therapeutic population of CISH^{lo}/PD-1^{lo} TILs, wherein the aldesleukin or a biosimilar or variant thereof is administered at a dose of 0.037 mg/kg or 0.044 mg/kg IU/kg (patient body mass) using 15-minute bolus intravenous infusions every eight hours until tolerance, for a maximum of 14 doses. Following 9 days of rest, this schedule may be repeated for another 14 doses, for a maximum of 28 doses in total. In some embodiments, IL-2 is administered in 1, 2, 3, 4, 5, or 6 doses. In some embodiments, IL-2 is administered at a maximum dosage of up to 6 doses.

In some embodiments, the IL-2 regimen comprises a decrescendo IL-2 regimen. Decrescendo IL-2 regimens have been described in O'Day, et al., J Clin. Oncol., 1999, 17, 2752-61 and Eton, et al., Cancer, 2000, 88, 1703-9, the disclosures of which are incorporated herein by reference. In some embodiments, a decrescendo IL-2 regimen comprises 18×10⁶ IU/m² administered intravenously over 6 hours, followed by 18×10⁶ IU/m² administered intravenously over 12 hours, followed by 18×10⁶ IU/m² administered intravenously over 24 hrs, followed by 4.5×10⁶ IU/m² administered intravenously over 72 hours. This treatment cycle may be repeated every 28 days for a maximum of four cycles. In some embodiments, a decrescendo IL-2 regimen comprises 18,000,000 IU/m² on day 1, 9,000,000 IU/m² on day 2, and 4,500,000 IU/m² on days 3 and 4.

In some embodiments, the IL-2 regimen comprises administration of pegylated IL-2 every 1, 2, 4, 6, 7, 14 or 21 days at a dose of 0.10 mg/day to 50 mg/day.

In some embodiments, the IL-2 regimen comprises administration of an IL-2 fragment engrafted onto an antibody backbone. In some embodiments, the IL-2 regimen comprises administration of an antibody-cytokine engrafted protein that binds the IL-2 low affinity receptor. In some embodiments, the antibody cytokine engrafted protein comprises a heavy chain variable region (VH), comprising complementarity determining regions HCDR1, HCDR2, HCDR3; a light chain variable region (VL), comprising LCDR1, LCDR2, LCDR3; and an IL-2 molecule or a fragment thereof engrafted into a CDR of the VH or the VL, wherein the antibody cytokine engrafted protein preferentially expands T effector cells over regulatory T cells. In some embodiments, the antibody cytokine engrafted protein comprises a heavy chain variable region (VH), comprising complementarity determining regions HCDR1, HCDR2, HCDR3; a light chain variable region (VL), comprising LCDR1, LCDR2, LCDR3; and an IL-2 molecule or a fragment thereof engrafted into a CDR of the VH or the VL, wherein the IL-2 molecule is a mutein, and wherein the antibody cytokine engrafted protein preferentially expands T effector cells over regulatory T cells. In some embodiments, the IL-2 regimen comprises administration of an antibody described in U.S. Patent Application Publication No. 2020/0270334 A1, the disclosures of which are incorporated by reference herein. In some embodiments, the antibody cytokine engrafted protein comprises a heavy chain variable region (VH), comprising complementarity determining regions HCDR1, HCDR2, HCDR3; a light chain variable region (VL), comprising LCDR1, LCDR2, LCDR3; and an IL-2 molecule or a fragment thereof engrafted into a CDR of the VH or the VL, wherein the IL-2 molecule is a mutein, wherein the antibody cytokine engrafted protein preferentially expands T effector cells over regulatory T cells, and wherein the antibody further comprises an IgG class heavy chain and an IgG class light chain selected from the group consisting of: a IgG class light chain comprising SEQ ID NO: 69 in U.S. Patent Application Publication No. 2020/0270334 A1 and a IgG class heavy chain comprising SEQ ID NO: 53 in U.S. Patent Application Publication No. 2020/0270334 A1; a IgG class light chain comprising SEQ ID NO: 37 in U.S. Patent Application Publication No. 2020/0270334 A1 and a IgG class heavy chain comprising SEQ ID NO: 21 in U.S. Patent Application Publication No. 2020/0270334 A1; a IgG class light chain comprising SEQ ID NO: 69 in U.S. Patent Application Publication No. 2020/0270334 A1 and a IgG class heavy chain comprising SEQ ID NO: 21 in U.S. Patent Application Publication No. 2020/0270334 A1; and a IgG class light chain comprising SEQ ID NO: 37 and a IgG class heavy chain comprising SEQ ID NO: 53 in U.S. Patent Application Publication No. 2020/0270334 A1.

In some embodiments, an IL-2 molecule or a fragment thereof is engrafted into HCDR1 of the VH, wherein the IL-2 molecule is a mutein. In some embodiments, an IL-2 molecule or a fragment thereof is engrafted into HCDR2 of the VH, wherein the IL-2 molecule is a mutein. In some embodiments, an IL-2 molecule or a fragment thereof is engrafted into HCDR3 of the VH, wherein the IL-2 molecule is a mutein. In some embodiments, an IL-2 molecule or a fragment thereof is engrafted into LCDR1 of the VL, wherein the IL-2 molecule is a mutein. In some embodiments, an IL-2 molecule or a fragment thereof is engrafted into LCDR2 of the VL, wherein the IL-2 molecule is a mutein. In some embodiments, an IL-2 molecule or a fragment thereof is engrafted into LCDR3 of the VL, wherein the IL-2 molecule is a mutein.

The insertion of the IL-2 molecule can be at or near the N-terminal region of the CDR, in the middle region of the CDR or at or near the C-terminal region of the CDR. In some embodiments, the antibody cytokine engrafted protein comprises an IL-2 molecule incorporated into a CDR, wherein the IL2 sequence does not frameshift the CDR sequence. In some embodiments, the antibody cytokine engrafted protein comprises an IL-2 molecule incorporated into a CDR, wherein the IL-2 sequence replaces all or part of a CDR sequence. The replacement by the IL-2 molecule can be the N-terminal region of the CDR, in the middle region of the CDR or at or near the C-terminal region the CDR. A replacement by the IL-2 molecule can be as few as one or two amino acids of a CDR sequence, or the entire CDR sequences

In some embodiments, an IL-2 molecule is engrafted directly into a CDR without a peptide linker, with no additional amino acids between the CDR sequence and the IL-2 sequence. In some embodiments, an IL-2 molecule is engrafted indirectly into a CDR with a peptide linker, with one or more additional amino acids between the CDR sequence and the IL-2 sequence.

In some embodiments, the IL-2 molecule described herein is an IL-2 mutein. In some instances, the IL-2 mutein comprising an R67A substitution. In some embodiments, the IL-2 mutein comprises the amino acid sequence SEQ ID NO: 4 or SEQ ID NO: 6 in U.S. Patent Application Publication No. 2020/0270334 A1. In some embodiments, the IL-2 mutein comprises an amino acid sequence in Table 1 in U.S. Patent Application Publication No. 2020/0270334 A1.

In some embodiments, the antibody cytokine engrafted protein comprises an HCDR1 selected from the group consisting of SEQ ID NO: 7, SEQ ID NO: 10, SEQ ID NO: 13 and SEQ ID NO: 16 of U.S. Patent Application Publication No. 2020/0270334 A1. In some embodiments, the antibody cytokine engrafted protein comprises an HCDR1 selected from the group consisting of SEQ ID NO: 7, SEQ ID NO: 10, SEQ ID NO: 13 and SEQ ID NO: 16, and an HCDR2 selected from the group consisting of SEQ ID NO: 8, SEQ ID NO: 11, SEQ ID NO: 14, and SEQ ID NO: 17 of U.S. Patent Application Publication No. 2020/0270334 A1. In some embodiments, the antibody cytokine engrafted protein comprises an HCDR1 selected from the group consisting of SEQ ID NO: 7, SEQ ID NO: 10, SEQ ID NO: 13 and SEQ ID NO: 16, an HCDR2 selected from the group consisting of SEQ ID NO: 8, SEQ ID NO: 11, SEQ ID NO: 14, and SEQ ID NO: 17, and an HCDR3 selected from the group consisting of SEQ ID NO: 9, SEQ ID NO: 12, SEQ ID NO: 15, and SEQ ID NO: 18 of U.S. Patent Application Publication No. 2020/0270334 A1. In some embodiments, the antibody cytokine engrafted protein comprises a VH region comprising the amino acid sequence of SEQ ID NO: 19 of U.S. Patent Application Publication No. 2020/0270334 A1. In some embodiments, the antibody cytokine engrafted protein comprises a heavy chain comprising the amino acid sequence of SEQ ID NO: 21 of U.S. Patent Application Publication No. 2020/0270334 A1. In some embodiments, the antibody cytokine engrafted protein comprises IgG.IL2R67A.H1 of U.S. Patent Application Publication No. 2020/0270334 A1. In some embodiments, the antibody components of the antibody cytokine engrafted protein described herein comprise immunoglobulin sequences, framework sequences, or CDR sequences of palivizumab.

In some embodiments, the antibody cytokine engrafted protein described herein has a longer serum half-life that a wild-type IL-2 molecule such as, but not limited to, aldeskeukin (Proleukin®) or a comparable molecule.

TABLE 5
Sequences of exemplary palivizumab antibody-IL-2 engrafted proteins.

| Identifier US | SEQ | |
|---|---|---|
| 2020/0270334 | ID NO: | Sequence |
| SEQ ID NO: 2 | SEQ ID | MYRMQLLSCI ALSLALVTNS APTSSSTKKT QLQLEHLLLD LQMILNGINN 50 |
| IL-2 | NO: 137 | YKNPKLTRML TFKFYMPKKA TELKHLQCLE EELKPLEEVL NLAQSKNFHL 100 |
| | IL-2 | RPRDLISNIN VIVLELKGSE TTFMCEYADE TATIVEFLNR WITFCQSIIS 150 |
| | | TLT 153 |
| SEQ ID NO: 4 | SEQ ID | APTSSSTKKT QLQLEHLLLD LQMILNGINN YKNPKLTAML TFKFYMPKKA 50 |
| IL-2 mutein | NO: 138 | TELKHLQCLE EELKPLEEVL NLAQSKNFHL RPRDLISNIN VIVLELKGSE 100 |
| | IL-2 | TTFMCEYADE TATIVEFLNR WITFCQSIIS TLT 133 |
| | mutein | |
| SEQ ID NO: 6 | SEQ ID | APTSSSTKKT QLQLEHLLLD LQMILNGINN YKNPKLTRML TAKFYMPKKA 50 |
| IL-2 mutein | NO: 139 | TELKHLQCLE EELKPLEEVL NLAQSKNFHL RPRDLISNIN VIVLELKGSE 100 |
| | IL-2 | TTFMCEYADE TATIVEFLNR WITFCQSIIS TLT 133 |
| | mutein | |
| IgG.IL2R67A.H1 | IgG.IL2R | |
| | 67A.H1 | |
| SEQ ID NO: 7 | SEQ ID | GFSLAPTSSS TKKTQLQLEH LLLDLQMILN GINNYKNPKL TAMLTFKFYM 50 |
| HCDR1_IL-2 | NO: 140 | PKKATELKHL QCLEEELKPL EEVLNLAQSK NFHLRPRDLI SNINVIVLEL 100 |
| | HCDR1_ | KGSETTFMCE YADETATIVE FLNRWITFCQ SIISTLTSTS GMSVG 145 |
| | IL-2 | |
| SEQ ID NO: 8 | SEQ ID | DIWWDDKKDY NPSLKS 16 |
| HCDR2 | NO: 141 | |
| | HCDR2 | |
| SEQ ID NO: 9 | SEQ ID | SMITNWYFDV 10 |
| HCDR3 | NO: 142 | |
| | HCDR3 | |
| SEQ ID NO: 10 | SEQ ID | TELKHLQCLE EELKPLEEVL NLAQSKNFHL RPRDLISNIN VIVLELKGSE 100 |
| HCDR1_IL-2 | NO: 143 | TTFMCEYADE TATIVEFLNR WITFCQSIIS TLTSTSGMSV G 141 |
| kabat | HCDR1_ | |
| | IL-2 kabat | |
| SEQ ID NO: 11 | SEQ ID | DIWWDDKKDY NPSLKS 16 |
| HCDR2 kabat | NO: 144 | |
| | HCDR2 | |
| | kabat | |
| SEQ ID NO: 12 | SEQ ID | SMITNWYFDV 10 |
| HCDR3 kabat | NO: 145 | |
| | HCDR3 | |
| | kabat | |
| SEQ ID NO: 13 | SEQ ID | GFSLAPTSSS TKKTQLQLEH LLLDLQMILN GINNYKNPKL TAMLTFKFYM 50 |
| HCDR1_IL-2 | NO: 146 | PKKATELKHL QCLEEELKPL EEVLNLAQSK NFHLRPRDLI SNINVIVLEL 100 |
| clothia | HCDR1_ | KGSETTFMCE YADETATIVE FLNRWITFCQ SIISTLTSTS GM 142 |
| | IL-2 | |
| | clothia | |
| SEQ ID NO: 14 | SEQ ID | WWDDK 5 |
| HCDR2 clothia | NO: 147 | |
| | HCDR2 | |
| | clothia | |
| SEQ ID NO: 15 | SEQ ID | SMITNWYFDV 10 |
| HCDR3 clothia | NO: 148 | |
| | HCDR3 | |
| | clothia | |

| | | |
|---|---|---|
| SEQ ID NO: 16 | SEQ ID | GFSLAPTSSS TKKTQLQLEH LLLDLQMILN GINNYKNPKL TAMLTFKFYM 50 |
| HCDR1_IL-2 | NO: 149 | PKKATELKHL QCLEEELKPL EEVLNLAQSK NFHLRPRDLI SNINVIVLEL 100 |
| IMGT | HCDR1_ | KGSETTFMCE YADETATIVE FLNRWITFCQ SIISTLTSTS GMS 143 |
| | IL-2 IMGT | |
| SEQ ID NO: 17 | SEQ ID | IWWDDKK 7 |
| HCDR2 IMGT | NO: 150 | |
| | HCDR2 | |
| | IMGT | |
| SEQ ID NO: 18 | SEQ ID | ARSMITNWYF DV 12 |
| HCDR3 IMGT | NO: 151 | |
| | HCDR3 | |
| | IMGT | |
| SEQ ID NO: 19 | SEQ ID | QVTLRESGPA LVKPTQTLTL TCTFSGFSLA PTSSSTKKTQ LQLEHLLLDL 50 |
| VH | NO: 152 | QMILNGINNY KNPKLTAMLT FKFYMPKKAT ELKHLQCLEE ELKPLEEVLN 100 |
| | VH | LAQSKNFHLR PRDLISNINV IVLELKGSET TFMCEYADET ATIVEFLNRW 150 |
| | | ITFCQSIIST LTSTSGMSVG WIRQPPGKAL EWLADIWWDD KKDYNPSLKS 200 |
| | | RLTISKDTSK NQVVLKVTNM DPADTATYYC ARSMITNWYF DVWGAGTTVT 250 |
| | | VSS 253 |
| SEQ ID NO: 21 | SEQ ID | QMILNGINNY KNPKLTAMLT FKFYMPKKAT ELKHLQCLEE ELKPLEEVLN 100 |
| Heavy chain | NO: 153 | LAQSKNFHLR PRDLISNINV IVLELKGSET TFMCEYADET ATIVEFLNRW 150 |
| | Heavy | ITFCQSIIST LTSTSGMSVG WIRQPPGKAL EWLADIWWDD KKDYNPSLKS 200 |
| | chain | RLTISKDTSK NQVVLKVTNM DPADTATYYC ARSMITNWYF DVWGAGTTVT 250 |
| | | VSSASTKGPS VFPLAPSSKS TSGGTAALGC LVKDYFPEPV TVSWNSGALT 300 |
| | | SGVHTFPAVL QSSGLYSLSS VVTVPSSSLG TQTYICNVNH KPSNTKVDKR 350 |
| | | VEPKSCDKTH TCPPCPAPEL LGGPSVFLFP PKPKDTLMIS RTPEVTCVVV 400 |
| | | AVSHEDPEVK FNWYVDGVEV HNAKTKPREE QYNSTYRVVS VLTVLHQDWL 450 |
| | | NGKEYKCKVS NKALAAPIEK TISKAKGQPR EPQVYTLPPS REEMTKNQVS 500 |
| | | LTCLVKGFYP SDIAVEWESN GQPENNYKTT PPVLDSDGSF FLYSKLTVDK 550 |
| | | SRWQQGNVFS CSVMHEALHN HYTQKSLSLS PGK 583 |
| SEQ ID NO: 26 | SEQ ID | KAQLSVGYMH 10 |
| LCDR1 kabat | NO: 154 | |
| | LCDR1 | |
| | kabat | |
| SEQ ID NO: 27 | SEQ ID | DTSKLAS 7 |
| LCDR2 kabat | NO: 155 | |
| | LCDR2 | |
| | kabat | |
| SEQ ID NO: 28 | SEQ ID | FQGSGYPFT 9 |
| LCDR3 kabat | NO: 156 | |
| | LCDR3 | |
| | kabat | |
| SEQ ID NO: 29 | SEQ ID | QLSVGY 6 |
| LCDR1 chothia | NO: 157 | |
| | LCDR1 | |
| | chothia | |
| SEQ ID NO: 30 | SEQ ID | DTS 3 |
| LCDR2 chothia | NO: 158 | |
| | LCDR2 | |
| | chothia | |
| SEQ ID NO: 31 | SEQ ID | GSGYPF 6 |
| LCDR3 chothia | NO: 159 | |
| | LCDR3 | |
| | chothia | |
| SEQ ID NO: 35 | SEQ ID | DIQMTQSPST LSASVGDRVT ITCKAQLSVG YMHWYQQKPG KAPKLLIYDT 50 |
| VL | NO: 160 | SKLASGVPSR FSGSGSGTEF TLTISSLQPD DFATYYCFQG SGYPFTFGGG 100 |
| | VL | TKLEIK 106 |
| SEQ ID NO: 37 | SEQ ID | DIQMTQSPST LSASVGDRVT ITCKAQLSVG YMHWYQQKPG KAPKLLIYDT 50 |
| Light chain | NO: 161 | SKLASGVPSR FSGSGSGTEF TLTISSLQPD DFATYYCFQG SGYPFTFGGG 100 |
| | Light | TKLEIKRTVA APSVFIFPPS DEQLKSGTAS VVCLLNNFYP REAKVQWKVD 150 |
| | chain | NALQSGNSQE SVTEQDSKDS TYSLSSTLTL SKADYEKHKV YACEVTHQGL 200 |
| | | SSPVTKSFNR GEC 213 |
| SEQ ID NO: 53 | SEQ ID | QVTLRESGPA LVKPTQTLTL TCTFSGFSLA PTSSSTKKTQ LQLEHLLLDL 50 |
| Light chain | NO: 162 | QMILNGINNY KNPKLTRMLT AKFYMPKKAT ELKHLQCLEE ELKPLEEVLN 100 |
| | Light | LAQSKNFHLR PRDLISNINV IVLELKGSET TFMCEYADET ATIVEFLNRW 150 |
| | chain | ITFCQSIIST LTSTSGMSVG WIRQPPGKAL EWLADIWWDD KKDYNPSLKS 200 |
| | | RLTISKDTSK NQVVLKVTNM DPADTATYYC ARSMITNWYF DVWGAGTTVT 250 |
| | | VSSASTKGPS VFPLAPSSKS TSGGTAALGC LVKDYFPEPV TVSWNSGALT 300 |
| | | SGVHTFPAVL QSSGLYSLSS VVTVPSSSLG TQTYICNVNH KPSNTKVDKR 350 |
| | | VEPKSCDKTH TCPPCPAPEL LGGPSVFLFP PKPKDTLMIS RTPEVTCVVV 400 |
| | | AVSHEDPEVK FNWYVDGVEV HNAKTKPREE QYNSTYRVVS VLTVLHQDWL 450 |
| | | NGKEYKCKVS NKALAAPIEK TISKAKGQPR EPQVYTLPPS REEMTKNQVS 500 |
| | | LTCLVKGFYP SDIAVEWESN GQPENNYKTT PPVLDSDGSF FLYSKLTVDK 550 |
| | | SRWQQGNVFS CSVMHEALHN HYTQKSLSLS PGK 583 |
| SEQ ID NO: 69 | SEQ ID | DIQMTQSPST LSASVGDRVT ITCKAQLSVG YMHWYQQKPG KAPKLLIYDT 50 |
| Light chain | NO: 163 | SKLASGVPSR FSGSGSGTEF TLTISSLQPD DFATYYCFQG SGYPFTFGGG 100 |
| | Light | TKLEIKRTVA APSVFIFPPS DEQLKSGTAS VVCLLNNFYP REAKVQWKVD 150 |
| | chain | NALQSGNSQE SVTEQDSKDS TYSLSSTLTL SKADYEKHKV YACEVTHQGL 200 |
| | | SSPVTKSFNR GEC 213 |

### 3. Additional Methods of Treatment

In other embodiments, the invention provides a method for treating a subject with cancer comprising administering to the subject a therapeutically effective dosage of the therapeutic CISH^{lo} or CISH^{lo}/PD-1^{lo} TIL population described in any of the preceding paragraphs above.

In other embodiments, the invention provides a method for treating a subject with cancer comprising administering to the subject a therapeutically effective dosage of the CISH^{lo} or CISH^{lo}/PD-1^{lo} TIL composition described in any of the preceding paragraphs above.

In other embodiments, the invention provides the method for treating a subject with cancer described in any of the preceding paragraphs above modified such that prior to administering the therapeutically effective dosage of the therapeutic population of CISH^{lo} or CISH^{lo}/PD-1^{lo} TILs and the CISH^{lo} or CISH^{lo}/PD-1^{lo} TIL composition described in any of the preceding paragraphs above, respectively, a non-myeloablative lymphodepletion regimen has been administered to the subject.

In other embodiments, the invention provides the method for treating a subject with cancer described in any of the preceding paragraphs above modified such that the non-myeloablative lymphodepletion regimen comprises the steps of administration of cyclophosphamide at a dose of 60 mg/m²/day for two days followed by administration of fludarabine at a dose of 25 mg/m²/day for five days.

In some embodiments, the invention provides the method for treating a subject with cancer described in any of the preceding paragraphs above modified such that the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for three days.

In some embodiments, the invention provides the method for treating a subject with cancer described in any of the preceding paragraphs above modified such that the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for one day.

In some embodiments, the invention provides the method for treating a subject with cancer described in any of the preceding paragraphs above modified such that the non-myeloablative lymphodepletion regimen comprises the steps of administration of cyclophosphamide at a dose of 60 mg/m²/day for two days followed by administration of fludarabine at a dose of 25 mg/m²/day for three days.

In other embodiments, the invention provides the method for treating a subject with cancer described in any of the preceding paragraphs above modified to further comprise the step of treating the subject with a high-dose IL-2 regimen starting on the day after administration of the TIL cells to the subject.

In other embodiments, the invention provides the method for treating a subject with cancer described in any of the preceding paragraphs above modified such that the high-dose IL-2 regimen comprises 600,000 or 720,000 IU/kg administered as a 15-minute bolus intravenous infusion every eight hours until tolerance.

In other embodiments, the invention provides the method for treating a subject with cancer described in any of the preceding paragraphs above modified such that the cancer is a solid tumor.

In other embodiments, the invention provides the method for treating a subject with cancer described in any of the preceding paragraphs above modified such that the cancer is melanoma.

In other embodiments, the invention provides the method for treating a subject with cancer described in any of the preceding paragraphs above modified such that the cancer is a pediatric hypermutated cancer.

In other embodiments, the invention provides the therapeutic TIL population of CISH^{lo} or CISH^{lo}/PD-1^{lo} TILs described in any of the preceding paragraphs above for use in a method for treating a subject with cancer comprising administering to the subject a therapeutically effective dosage of the therapeutic CISH^{lo} or CISH^{lo}/PD-1^{lo} TIL population.

In other embodiments, the invention provides the CISH^{lo} or CISH^{lo}/PD-1^{lo} TIL composition described in any of the preceding paragraphs above for use in a method for treating a subject with cancer comprising administering to the subject a therapeutically effective dosage of the TIL composition.

In other embodiments, the invention provides the therapeutic CISH^{lo} or CISH^{lo}/PD-1^{lo} TIL population described in any of the preceding paragraphs above or the CISH^{lo} or CISH^{lo}/PD-1^{lo} TIL composition described in any of the preceding paragraphs above modified such that prior to administering to the subject the therapeutically effective dosage of the therapeutic CISH^{lo} or CISH^{lo}/PD-1^{lo} TIL population described in any of the preceding paragraphs above or the CISH^{lo} or CISH^{lo}/PD-1^{lo} TIL composition described in any of the preceding paragraphs above, a non-myeloablative lymphodepletion regimen has been administered to the subject.

In other embodiments, the invention provides the therapeutic CISH^{lo} or CISH^{lo}/PD-1^{lo} TIL population or the CISH^{lo} or CISH^{lo}/PD-1^{lo} TIL composition described in any of the preceding paragraphs above modified such that the non-myeloablative lymphodepletion regimen comprises the steps of administration of cyclophosphamide at a dose of 60 mg/m²/day for two days followed by administration of fludarabine at a dose of 25 mg/m²/day for five days.

In some embodiments, the invention provides the therapeutic CISH^{lo} or CISH^{lo}/PD-1^{lo} TIL population or the CISH^{lo} or CISH^{lo}/PD-1^{lo} TIL composition described in any of the preceding paragraphs above modified such that the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for three days.

In some embodiments, the invention provides the therapeutic CISH^{lo} or CISH^{lo}/PD-1^{lo} TIL population or the CISH^{lo} or CISH^{lo}/PD-1^{lo} TIL composition described in any of the preceding paragraphs above modified such that the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for one day.

In some embodiments, the invention provides the therapeutic CISH^{lo} or CISH^{lo}/PD-1^{lo} TIL population or the CISH^{lo} or CISH^{lo}/PD-1^{lo} TIL composition described in any of the preceding paragraphs above modified such that the non-myeloablative lymphodepletion regimen comprises the steps of administration of cyclophosphamide at a dose of 60 mg/m²/day for two days followed by administration of fludarabine at a dose of 25 mg/m²/day for three days.

In other embodiments, the invention provides the therapeutic CISH^{lo} or CISH^{lo}/PD-1^{lo} TIL population or the CISH^{lo} or CISH^{lo}/PD-1^{lo} TIL composition described in any of the preceding paragraphs above modified to further comprise the step of treating patient with a high-dose IL-2 regimen starting on the day after administration of the TIL cells to the patient.

In other embodiments, the invention provides the therapeutic CISH^{lo} or CISH^{lo}/PD-1^{lo} TIL population or the CISH^{lo} or CISH^{lo}/PD-1^{lo} TIL composition described in any of the preceding paragraphs above modified such that the high-dose IL-2 regimen comprises 600,000 or 720,000 IU/kg administered as a 15-minute bolus intravenous infusion every eight hours until tolerance.

In other embodiments, the invention provides the therapeutic CISH^{lo} or CISH^{lo}/PD-1^{lo} TIL population or the CISH^{lo} or CISH^{lo}/PD-1^{lo} TIL composition described in any of the preceding paragraphs above modified such that the cancer is a solid tumor.

In other embodiments, the invention provides the therapeutic CISH^{lo} or CISH^{lo}/PD-1^{lo} TIL population or the CISH^{lo} or CISH^{lo}/PD-1^{lo} TIL composition described in any of the preceding paragraphs above modified such that the cancer is melanoma.

In other embodiments, the invention provides the therapeutic CISH^{lo} or CISH¹⁰/PD-1^{lo} TIL population or the CISH^{lo} or CISH^{lo}/PD-1^{lo} TIL composition described in any of the preceding paragraphs above modified such that the cancer is a hypermutated cancer.

In other embodiments, the invention provides the therapeutic CISH^{lo} or CISH¹⁰/PD-1^{lo} TIL population or the CISH^{lo} or CISH^{lo}/PD-1^{lo} TIL composition described in any of the preceding paragraphs above modified such that the cancer is a pediatric hypermutated cancer.

In other embodiments, the invention provides the use of the therapeutic CISH^{lo} or CISH^{lo}/PD-1^{lo} TIL population described in any of any of the preceding paragraphs above in a method of treating cancer in a subject comprising administering to the subject a therapeutically effective dosage of the therapeutic CISH^{lo} or CISH^{lo}/PD-1^{lo} TIL population.

In other embodiments, the invention provides the use of the CISH^{lo} or CISH^{lo}/PD-1^{lo} TIL composition described in any of the preceding paragraphs above in a method of treating cancer in a subject comprising administering to the subject a therapeutically effective dosage of the CISH^{lo} or CISH¹⁰/PD-1^{lo} TIL composition.

In other embodiments, the invention provides the use of the therapeutic CISH^{lo} or CISH^{lo}/PD-1^{lo} TIL population described in any of the preceding paragraphs above or the CISH^{lo} or CISH¹⁰/PD-1^{lo} TIL composition described in any of the preceding paragraphs above in a method of treating cancer in a subject comprising administering to the subject a non-myeloablative lymphodepletion regimen and then administering to the subject the therapeutically effective dosage of the therapeutic CISH^{lo} or CISH^{lo}/PD-1^{lo} TIL population described in any of the preceding paragraphs above or the therapeutically effective dosage of the CISH^{lo} or CISH^{lo}/PD-1^{lo} TIL composition described in any of the preceding paragraphs above.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Preparation of Tils with CISH Knockout

This Example describes the procedure for the preparation of tumor infiltrating lymphocytes with CISH knockout (CISH KO TIL). This media can be used for preparation of any of the TILs described in the present application and Examples.

### Protocol for CISH KO TIL Expansion

Pre-Expansion set up: Pre-REP cultures were initiated from 6 to 8 tumor fragments per G-REX 10 flask in CM1 with IL-2 for 11 days. Pre-REP TIL were cryopreserved in CS10 freezing media at 35e6 cells per vial and kept at −80° C. until use.

Pre-Expansion TIL thaw: In exemplary cases where the TILs were cryopreserved, the cryopreserved TIL were thawed and rested in CM1 containing IL-2 (3000 IU/mL) at 2e6 cells per well in 24-well plate for two days.

T-cell activation: The cells were activated with plate-bound anti-CD3 at concentration of 300 ng/ml for another two days.

Electroporation: TIL were electroporated with CISH TALEN-encoding mRNAs, PD-1 TALEN-encoding mRNAs, CISH TALEN-encoding mRNAs+PD-1 TALEN-encoding mRNAs, or non-electroporated. For each electroporation, one million activated TIL were washed twice with Cytoporation buffer T4. The cells were resuspended in 50 ul of Cytoporation T4 buffer containing 4 μg of TALEN mRNA for each arm. The cells were transferred into a 1 mm Gap electroporation cuvette and electroporated using BTX AgilePulse. Immediately after the electroporation, the cells were resuspended in 1 ml of CM1 media and plated in a 24-well plate well. The cells were incubated at 37° C. for an hour, followed by 30° C. for 15 hours.

Expansion: Non-electroporated and CISH KO TALEN mRNA electroporated TIL (1e5 cells) were expanded using a rapid expansion protocol (REP) by culturing in OKT3 (30 ng/ml, Miltenyi Biotec), IL-2 (6000 IU/ml, CellGenix) and irradiated PBMCs (30e6 cells) for 11 days.

Post-Expansion TIL harvest: The cells were harvested and treated as follows.

Post-Expansion TILs were re-stimulated with anti-CD3 overnight prior to being assessed for CISH protein by western blot analysis and/or assessed for PD-1 expression by flow cytometry. NE=non-electroporation; 293T cells overexpressing CISH proteins were used as a positive control; Densitometry data from western blot analysis were used for calculation of relative fold change; NE was used for baseline calculation.

TABLE 6
Assessment of Double KO in Post-Expansion TIL

| Category | Method |
|---|---|
| Viability | Automated cell counter |
| Fold expansion | Calculation (multiplication of # total |
| | number of cell count on D16 and # of |
| | seeded cells ) |
| Phenotypic characterization | Flow cytometry |
| (Differentiation, Memory | |
| T-cell subset, and T-cell | |
| activation/exhaustion) | |
| KO efficiency | Flow cytometry (PD-1) and western blot |
| | (CISH) |
| Effector function | ELISA |
| (IFN-gamma secretion | |
| upon re-stimulation | |
| with aCD3) | |

Description of the CISH KO TALEN-encoding sequences used in the experiments and its corresponding cleavage site in the CISH gene is provided in Table 7 below.

TABLE 7
Description of CISH KO TALE-nucleases used in the experiments and
sequences of the TALE-nuclease cleavage site in the human CISH gene

| Sequence | | |
|---|---|---|
| name | Ref. Sequences | Polynucleotide or polypeptide sequences |
| Left CISH KO | SEQ ID NO: 164 | ATGGGCGATCCTAAAAAGAAACGTAAGGTCATCGATATCGCCGATC |
| TALEN | | TACGCACGCTCGGCTACAGCCAGCAGCAACAGGAGAAGATCAAACC |
| pCLS34485 | | GAAGGTTCGTTCGACAGTGGCGCAGCACCACGAGGCACTGGTCGGC |
| CISHe3_9-L1 | | CACGGGTTTACACACGCGCACATCGTTGCGTTAAGCCAACACCCGG |
| Nucleotide | | CAGCGTTAGGGACCGTCGCTGTCAAGTATCAGGACATGATCGCAGC |
| sequence | | GTTGCCAGAGGCGACACACGAAGCGATCGTTGGCGTCGGCAAACAG |
| | | TGGTCCGGCGCACGCGCTCTGGAGGCCTTGCTCACGGTGGCGGGAG |
| | | AGTTGAGAGGTCCACCGTTACAGTTGGACACAGGCCAACTTCTCAA |
| | | GATTGCAAAACGTGGCGGCGTGACCGCAGTGGAGGCAGTGCATGCA |
| | | TGGCGCAATGCACTGACGGGTGCCCCGCTCAACTTGACCCCCCAGC |
| | | AGGTGGTGGCCATCGCCAGCAATAATGGTGGCAAGCAGGCGCTGGA |
| | | GACGGTCCAGCGGCTGTTGCCGGTGCTGTGCCAGGCCCACGGCTTG |
| | | ACCCCGGAGCAGGTGGTGGCCATCGCCAGCCACGATGGCGGCAAGC |
| | | AGGCGCTGGAGACGGTCCAGCGGCTGTTGCCGGTGCTGTGCCAGGC |
| | | CCACGGCTTGACCCCCCAGCAGGTGGTGGCCATCGCCAGCAATAAT |
| | | GGTGGCAAGCAGGCGCTGGAGACGGTCCAGCGGCTGTTGCCGGTGC |
| | | TGTGCCAGGCCCACGGCTTGACCCCGGAGCAGGTGGTGGCCATCGC |
| | | CAGCCACGATGGCGGCAAGCAGGCGCTGGAGACGGTCCAGCGGCTG |
| | | TTGCCGGTGCTGTGCCAGGCCCACGGCTTGACCCCGGAGCAGGTGG |
| | | TGGCCATCGCCAGCCACGATGGCGGCAAGCAGGCGCTGGAGACGGT |
| | | CCAGCGGCTGTTGCCGGTGCTGTGCCAGGCCCACGGCTTGACCCCC |
| | | CAGCAGGTGGTGGCCATCGCCAGCAATGGCGGTGGCAAGCAGGCGC |
| | | TGGAGACGGTCCAGCGGCTGTTGCCGGTGCTGTGCCAGGCCCACGG |
| | | CTTGACCCCGGAGCAGGTGGTGGCCATCGCCAGCAATATTGGTGGC |
| | | AAGCAGGCGCTGGAGACGGTGCAGGCGCTGTTGCCGGTGCTGTGCC |
| | | AGGCCCACGGCTTGACCCCCCAGCAGGTGGTGGCCATCGCCAGCAA |
| | | TAATGGTGGCAAGCAGGCGCTGGAGACGGTCCAGCGGCTGTTGCCG |
| | | GTGCTGTGCCAGGCCCACGGCTTGACCCCCCAGCAGGTGGTGGCCA |
| | | TCGCCAGCAATGGCGGTGGCAAGCAGGCGCTGGAGACGGTCCAGCG |
| | | GCTGTTGCCGGTGCTGTGCCAGGCCCACGGCTTGACCCCCCAGCAG |
| | | GTGGTGGCCATCGCCAGCAATAATGGTGGCAAGCAGGCGCTGGAGA |
| | | CGGTCCAGCGGCTGTTGCCGGTGCTGTGCCAGGCCCACGGCTTGAC |
| | | CCCGGAGCAGGTGGTGGCCATCGCCAGCAATATTGGTGGCAAGCAG |
| | | GCGCTGGAGACGGTGCAGGCGCTGTTGCCGGTGCTGTGCCAGGCCC |
| | | ACGGCTTGACCCCGGAGCAGGTGGTGGCCATCGCCAGCCACGATGG |
| | | CGGCAAGCAGGCGCTGGAGACGGTCCAGCGGCTGTTGCCGGTGCTG |
| | | TGCCAGGCCCACGGCTTGACCCCGGAGCAGGTGGTGGCCATCGCCA |
| | | GCCACGATGGCGGCAAGCAGGCGCTGGAGACGGTCCAGCGGCTGTT |
| | | GCCGGTGCTGTGCCAGGCCCACGGCTTGACCCCGGAGCAGGTGGTG |
| | | GCCATCGCCAGCCACGATGGCGGCAAGCAGGCGCTGGAGACGGTCC |
| | | AGCGGCTGTTGCCGGTGCTGTGCCAGGCCCACGGCTTGACCCCGGA |
| | | GCAGGTGGTGGCCATCGCCAGCAATATTGGTGGCAAGCAGGCGCTG |
| | | GAGACGGTGCAGGCGCTGTTGCCGGTGCTGTGCCAGGCCCACGGCT |
| | | TGACCCCTCAGCAGGTGGTGGCCATCGCCAGCAATGGCGGCGGCAG |
| | | GCCGGCGCTGGAGAGCATTGTTGCCCAGTTATCTCGCCCTGATCCG |
| | | GCGTTGGCCGCGTTGACCAACGACCACCTCGTCGCCTTGGCCTGCC |
| | | TCGGCGGGCGTCCTGCGCTGGATGCAGTGAAAAAGGGATTGGGGGA |
| | | TCCTATCAGCCGTTCCCAGCTGGTGAAGTCCGAGCTGGAGGAGAAG |
| | | AAATCCGAGTTGAGGCACAAGCTGAAGTACGTGCCCCACGAGTACA |
| | | TCGAGCTGATCGAGATCGCCCGGAACAGCACCCAGGACCGTATCCT |
| | | GGAGATGAAGGTGATGGAGTTCTTCATGAAGGTGTACGGCTACAGG |
| | | GGCAAGCACCTGGGCGGCTCCAGGAAGCCCGACGGCGCCATCTACA |
| | | CCGTGGGCTCCCCCATCGACTACGGCGTGATCGTGGACACCAAGGC |
| | | CTACTCCGGCGGCTACAACCTGCCCATCGGCCAGGCCGACGAAATG |
| | | CAGAGGTACGTGGAGGAGAACCAGACCAGGAACAAGCACATCAACC |
| | | CCAACGAGTGGTGGAAGGTGTACCCCTCCAGCGTGACCGAGTTCAA |
| | | GTTCCTGTTCGTGTCCGGCCACTTCAAGGGCAACTACAAGGCCCAG |
| | | CTGACCAGGCTGAACCACATCACCAACTGCAACGGCGCCGTGCTGT |
| | | CCGTGGAGGAGCTCCTGATCGGCGGCGAGATGATCAAGGCCGGCAC |
| | | CCTGACCCTGGAGGAGGTGAGGAGGAAGTTCAACAACGGCGAGATC |
| | | AACTTCGCGGCCGACTGATAA |
| Left CISH KO | SEQ ID NO: 165 | MGDPKKKRKVIDIADLRTLGYSQQQQEKIKPKVRSTVAQHHEALVG |
| TALEN | | HGFTHAHIVALSQHPAALGTVAVKYQDMIAALPEATHEAIVGVGKQ |
| pCLS34485 | | WSGARALEALLTVAGELRGPPLQLDTGQLLKIAKRGGVTAVEAVHA |
| CISHe3_9-L1 | | WRNALTGAPLNLTPQQVVAIASNNGGKQALETVQRLLPVLCQAHGL |
| Amino acid | | TPEQVVAIASHDGGKQALETVQRLLPVLCQAHGLTPQQVVAIASNN |
| sequence | | GGKQALETVQRLLPVLCQAHGLTPEQVVAIASHDGGKQALETVQRL |
| | | LPVLCQAHGLTPEQVVAIASHDGGKQALETVQRLLPVLCQAHGLTP |
| | | QQVVAIASNGGGKQALETVQRLLPVLCQAHGLTPEQVVAIASNIGG |
| | | KQALETVQALLPVLCQAHGLTPQQVVAIASNNGGKQALETVQRLLP |
| | | VLCQAHGLTPQQVVAIASNGGGKQALETVQRLLPVLCQAHGLTPQQ |
| | | VVAIASNNGGKQALETVQRLLPVLCQAHGLTPEQVVAIASNIGGKQ |
| | | ALETVQALLPVLCQAHGLTPEQVVAIASHDGGKQALETVQRLLPVL |
| | | CQAHGLTPEQVVAIASHDGGKQALETVQRLLPVLCQAHGLTPEQVV |
| | | AIASHDGGKQALETVQRLLPVLCQAHGLTPEQVVAIASNIGGKQAL |
| | | ETVQALLPVLCQAHGLTPQQVVAIASNGGGRPALESIVAQLSRPDP |
| | | ALAALTNDHLVALACLGGRPALDAVKKGLGDPISRSQLVKSELEEK |
| | | KSELRHKLKYVPHEYIELIEIARNSTQDRILEMKVMEFFMKVYGYR |
| | | GKHLGGSRKPDGAIYTVGSPIDYGVIVDTKAYSGGYNLPIGQADEM |
| | | QRYVEENQTRNKHINPNEWWKVYPSSVTEFKFLFVSGHFKGNYKAQ |
| | | LTRLNHITNCNGAVLSVEELLIGGEMIKAGTLTLEEVRRKENNGEI |
| | | NFAAD |
| Right CISH KO | SEQ ID NO: 166 | ATGGGCGATCCTAAAAAGAAACGTAAGGTCATCGATATCGCCGATC |
| TALEN | | TACGCACGCTCGGCTACAGCCAGCAGCAACAGGAGAAGATCAAACC |
| pCLS34486 | | GAAGGTTCGTTCGACAGTGGCGCAGCACCACGAGGCACTGGTCGGC |
| CISHe3_9-R1 | | CACGGGTTTACACACGCGCACATCGTTGCGTTAAGCCAACACCCGG |
| Nucleotide | | CAGCGTTAGGGACCGTCGCTGTCAAGTATCAGGACATGATCGCAGC |
| sequence | | GTTGCCAGAGGCGACACACGAAGCGATCGTTGGCGTCGGCAAACAG |
| | | TGGTCCGGCGCACGCGCTCTGGAGGCCTTGCTCACGGTGGCGGGAG |
| | | AGTTGAGAGGTCCACCGTTACAGTTGGACACAGGCCAACTTCTCAA |
| | | GATTGCAAAACGTGGCGGCGTGACCGCAGTGGAGGCAGTGCATGCA |
| | | TGGCGCAATGCACTGACGGGTGCCCCGCTCAACTTGACCCCGGAGC |
| | | AGGTGGTGGCCATCGCCAGCAATATTGGTGGCAAGCAGGCGCTGGA |
| | | GACGGTGCAGGCGCTGTTGCCGGTGCTGTGCCAGGCCCACGGCTTG |
| | | ACCCCGGAGCAGGTGGTGGCCATCGCCAGCCACGATGGCGGCAAGC |
| | | AGGCGCTGGAGACGGTCCAGCGGCTGTTGCCGGTGCTGTGCCAGGC |
| | | CCACGGCTTGACCCCGGAGCAGGTGGTGGCCATCGCCAGCAATATT |
| | | GGTGGCAAGCAGGCGCTGGAGACGGTGCAGGCGCTGTTGCCGGTGC |
| | | TGTGCCAGGCCCACGGCTTGACCCCCCAGCAGGTGGTGGCCATCGC |
| | | CAGCAATAATGGTGGCAAGCAGGCGCTGGAGACGGTCCAGCGGCTG |
| | | TTGCCGGTGCTGTGCCAGGCCCACGGCTTGACCCCGGAGCAGGTGG |
| | | TGGCCATCGCCAGCCACGATGGCGGCAAGCAGGCGCTGGAGACGGT |
| | | CCAGCGGCTGTTGCCGGTGCTGTGCCAGGCCCACGGCTTGACCCCG |
| | | GAGCAGGTGGTGGCCATCGCCAGCAATATTGGTGGCAAGCAGGCGC |
| | | TGGAGACGGTGCAGGCGCTGTTGCCGGTGCTGTGCCAGGCCCACGG |
| | | CTTGACCCCCCAGCAGGTGGTGGCCATCGCCAGCAATAATGGTGGC |
| | | AAGCAGGCGCTGGAGACGGTCCAGCGGCTGTTGCCGGTGCTGTGCC |
| | | AGGCCCACGGCTTGACCCCCCAGCAGGTGGTGGCCATCGCCAGCAA |
| | | TGGCGGTGGCAAGCAGGCGCTGGAGACGGTCCAGCGGCTGTTGCCG |
| | | GTGCTGTGCCAGGCCCACGGCTTGACCCCCCAGCAGGTGGTGGCCA |
| | | TCGCCAGCAATAATGGTGGCAAGCAGGCGCTGGAGACGGTCCAGCG |
| | | GCTGTTGCCGGTGCTGTGCCAGGCCCACGGCTTGACCCCCCAGCAG |
| | | GTGGTGGCCATCGCCAGCAATAATGGTGGCAAGCAGGCGCTGGAGA |
| | | CGGTCCAGCGGCTGTTGCCGGTGCTGTGCCAGGCCCACGGCTTGAC |
| | | CCCGGAGCAGGTGGTGGCCATCGCCAGCCACGATGGCGGCAAGCAG |
| | | GCGCTGGAGACGGTCCAGCGGCTGTTGCCGGTGCTGTGCCAGGCCC |
| | | ACGGCTTGACCCCCCAGCAGGTGGTGGCCATCGCCAGCAATGGCGG |
| | | TGGCAAGCAGGCGCTGGAGACGGTCCAGCGGCTGTTGCCGGTGCTG |
| | | TGCCAGGCCCACGGCTTGACCCCCCAGCAGGTGGTGGCCATCGCCA |
| | | GCAATAATGGTGGCAAGCAGGCGCTGGAGACGGTCCAGCGGCTGTT |
| | | GCCGGTGCTGTGCCAGGCCCACGGCTTGACCCCCCAGCAGGTGGTG |
| | | GCCATCGCCAGCAATAATGGTGGCAAGCAGGCGCTGGAGACGGTCC |
| | | AGCGGCTGTTGCCGGTGCTGTGCCAGGCCCACGGCTTGACCCCCCA |
| | | GCAGGTGGTGGCCATCGCCAGCAATGGCGGTGGCAAGCAGGCGCTG |
| | | GAGACGGTCCAGCGGCTGTTGCCGGTGCTGTGCCAGGCCCACGGCT |
| | | TGACCCCTCAGCAGGTGGTGGCCATCGCCAGCAATGGCGGCGGCAG |
| | | GCCGGCGCTGGAGAGCATTGTTGCCCAGTTATCTCGCCCTGATCCG |
| | | GCGTTGGCCGCGTTGACCAACGACCACCTCGTCGCCTTGGCCTGCC |
| | | TCGGCGGGCGTCCTGCGCTGGATGCAGTGAAAAAGGGATTGGGGGA |
| | | TCCTATCAGCCGTTCCCAGCTGGTGAAGTCCGAGCTGGAGGAGAAG |
| | | AAATCCGAGTTGAGGCACAAGCTGAAGTACGTGCCCCACGAGTACA |
| | | TCGAGCTGATCGAGATCGCCCGGAACAGCACCCAGGACCGTATCCT |
| | | GGAGATGAAGGTGATGGAGTTCTTCATGAAGGTGTACGGCTACAGG |
| | | GGCAAGCACCTGGGCGGCTCCAGGAAGCCCGACGGCGCCATCTACA |
| | | CCGTGGGCTCCCCCATCGACTACGGCGTGATCGTGGACACCAAGGC |
| | | CTACTCCGGCGGCTACAACCTGCCCATCGGCCAGGCCGACGAAATG |
| | | CAGAGGTACGTGGAGGAGAACCAGACCAGGAACAAGCACATCAACC |
| | | CCAACGAGTGGTGGAAGGTGTACCCCTCCAGCGTGACCGAGTTCAA |
| | | GTTCCTGTTCGTGTCCGGCCACTTCAAGGGCAACTACAAGGCCCAG |
| | | CTGACCAGGCTGAACCACATCACCAACTGCAACGGCGCCGTGCTGT |
| | | CCGTGGAGGAGCTCCTGATCGGCGGCGAGATGATCAAGGCCGGCAC |
| | | CCTGACCCTGGAGGAGGTGAGGAGGAAGTTCAACAACGGCGAGATC |
| | | AACTTCGCGGCCGACTGATAA |
| Right CISH KO | SEQ ID NO: 167 | MGDPKKKRKVIDIADLRTLGYSQQQQEKIKPKVRSTVAQHHEALVG |
| TALEN | | HGFTHAHIVALSQHPAALGTVAVKYQDMIAALPEATHEAIVGVGKQ |
| pCLS34486 | | WSGARALEALLTVAGELRGPPLQLDTGQLLKIAKRGGVTAVEAVHA |
| CISHe3_9-R1 | | WRNALTGAPLNLTPEQVVAIASNIGGKQALETVQALLPVLCQAHGL |
| Amino acid | | TPEQVVAIASHDGGKQALETVQRLLPVLCQAHGLTPEQVVAIASNI |
| sequence | | GGKQALETVQALLPVLCQAHGLTPQQVVAIASNNGGKQALETVQRL |
| | | LPVLCQAHGLTPEQVVAIASHDGGKQALETVQRLLPVLCQAHGLTP |
| | | EQVVAIASNIGGKQALETVQALLPVLCQAHGLTPQQVVAIASNNGG |
| | | KQALETVQRLLPVLCQAHGLTPQQVVAIASNGGGKQALETVQRLLP |
| | | VLCQAHGLTPQQVVAIASNNGGKQALETVQRLLPVLCQAHGLTPQQ |
| | | VVAIASNNGGKQALETVQRLLPVLCQAHGLTPEQVVAIASHDGGKQ |
| | | ALETVQRLLPVLCQAHGLTPQQVVAIASNGGGKQALETVQRLLPVL |
| | | CQAHGLTPQQVVAIASNNGGKQALETVQRLLPVLCQAHGLTPQQVV |
| | | AIASNNGGKQALETVQRLLPVLCQAHGLTPQQVVAIASNGGGKQAL |
| | | ETVQRLLPVLCQAHGLTPQQVVAIASNGGGRPALESIVAQLSRPDP |
| | | ALAALTNDHLVALACLGGRPALDAVKKGLGDPISRSQLVKSELEEK |
| | | KSELRHKLKYVPHEYIELIEIARNSTQDRILEMKVMEFFMKVYGYR |
| | | GKHLGGSRKPDGAIYTVGSPIDYGVIVDTKAYSGGYNLPIGQADEM |
| | | QRYVEENQTRNKHINPNEWWKVYPSSVTEFKFLFVSGHFKGNYKAQ |
| | | LTRLNHITNCNGAVLSVEELLIGGEMIKAGTLTLEEVRRKFNNGEI |
| | | NFAAD |
| CISH KO | SEQ ID NO: 168 | TGCGCCTAGTGACCCAGCACTGCCTGCTCCTCCACCAGCCACTGCT |
| Cleavage Site | | GTA |
| sequence | | |
| CISHe3_9.1 | | |
| Nucleotide | | |
| sequence | | |

Description of the PD-1 KO TALEN used in the experiments and its corresponding cleavage site in the PD-1 gene is provided in Table 8 below.

TABLE 8
Description of PD-1 KO TALE-nucleases used in the experiments and
sequences of the TALE-nuclease cleavage site in the human PD-1 gene

| Sequence | Ref. | |
|---|---|---|
| name | Sequences | Polynucleotide or polypeptide sequences |
| Left PD-1 | SEQ ID | ATGGGCGATCCTAAAAAGAAACGTAAGGTCATCGATATCGCCGATCTACGCAC |
| KO TALEN | NO: 169 | GCTCGGCTACAGCCAGCAGCAACAGGAGAAGATCAAACCGAAGGTTCGTTCGA |
| mRNA | | CAGTGGCGCAGCACCACGAGGCACTGGTCGGCCACGGGTTTACACACGCGCAC |
| sequence | | ATCGTTGCGTTAAGCCAACACCCGGCAGCGTTAGGGACCGTCGCTGTCAAGTA |
| (pCLS29259) | | TCAGGACATGATCGCAGCGTTGCCAGAGGCGACACACGAAGCGATCGTTGGCG |
| | | TCGGCAAACAGTGGTCCGGCGCACGCGCTCTGGAGGCCTTGCTCACGGTGGCG |
| | | GGAGAGTTGAGAGGTCCACCGTTACAGTTGGACACAGGCCAACTTCTCAAGAT |
| | | TGCAAAACGTGGCGGCGTGACCGCAGTGGAGGCAGTGCATGCATGGCGCAATG |
| | | CACTGACGGGTGCCCCGCTCAACTTGACCCCCGAGCAAGTGGTGGCTATCGCT |
| | | TCCAAGCTGGGGGGAAAGCAGGCCCTGGAGACCGTCCAGGCCCTTCTCCCAGT |
| | | GCTTTGCCAGGCTCACGGACTGACCCCTGAACAGGTGGTGGCAATTGCCTCAC |
| | | ACGACGGGGGCAAGCAGGCACTGGAGACTGTCCAGCGGCTGCTGCCTGTCCTC |
| | | TGCCAGGCCCACGGACTCACTCCTGAGCAGGTCGTGGCCATTGCCAGCCACGA |
| | | TGGGGGCAAACAGGCTCTGGAGACCGTGCAGCGCCTCCTCCCAGTGCTGTGCC |
| | | AGGCTCATGGGCTGACCCCACAGCAGGTCGTCGCCATTGCCAGTAACGGCGGG |
| | | GGGAAGCAGGCCCTCGAAACAGTGCAGAGGCTGCTGCCCGTCTTGTGCCAAGC |
| | | ACACGGCCTGACACCCGAGCAGGTGGTGGCCATCGCCTCTCATGACGGCGGCA |
| | | AGCAGGCCCTTGAGACAGTGCAGAGACTGTTGCCCGTGTTGTGTCAGGCCCAC |
| | | GGGTTGACACCCCAGCAGGTGGTCGCCATCGCCAGCAATGGCGGGGGAAAGCA |
| | | GGCCCTTGAGACCGTGCAGCGGTTGCTTCCAGTGTTGTGCCAGGCACACGGAC |
| | | TGACCCCTCAACAGGTGGTCGCAATCGCCAGCTACAAGGGCGGAAAGCAGGCT |
| | | CTGGAGACAGTGCAGCGCCTCCTGCCCGTGCTGTGTCAGGCTCACGGACTGAC |
| | | ACCACAGCAGGTGGTCGCCATCGCCAGTAACGGGGGCGGCAAGCAGGCTTTGG |
| | | AGACCGTCCAGAGACTCCTCCCCGTCCTTTGCCAGGCCCACGGGTTGACACCT |
| | | CAGCAGGTCGTCGCCATTGCCTCCAACAACGGGGGCAAGCAGGCCCTCGAAAC |
| | | TGTGCAGAGGCTGCTGCCTGTGCTGTGCCAGGCTCATGGGCTGACACCCCAGC |
| | | AGGTGGTGGCCATTGCCTCTAACAACGGCGGCAAACAGGCACTGGAGACCGTG |
| | | CAAAGGCTGCTGCCCGTCCTCTGCCAAGCCCACGGGCTCACTCCACAGCAGGT |
| | | CGTGGCCATCGCCTCAAACAATGGCGGGAAGCAGGCCCTGGAGACTGTGCAAA |
| | | GGCTGCTCCCTGTGCTCTGCCAGGCACACGGACTGACCCCTCAGCAGGTGGTG |
| | | GCAATCGCTTCCAACAACGGGGGAAAGCAGGCCCTCGAAACCGTGCAGCGCCT |
| | | CCTCCCAGTGCTGTGCCAGGCACATGGCCTCACACCCGAGCAAGTGGTGGCTA |
| | | TCGCCAGCCACGACGGAGGGAAGCAGGCTCTGGAGACCGTGCAGAGGCTGCTG |
| | | CCTGTCCTGTGCCAGGCCCACGGGCTTACTCCAGAGCAGGTCGTCGCCATCGC |
| | | CAGTCATGATGGGGGGAAGCAGGCCCTTGAGACAGTCCAGCGGCTGCTGCCAG |
| | | TCCTTTGCCAGGCTCACGGCTTGACTCCCGAGCAGGTCGTGGCCATTGCCTCA |
| | | AACATTGGGGGCAAACAGGCCCTGGAGACAGTGCAGGCCCTGCTGCCCGTGTT |
| | | GTGTCAGGCCCACGGCTTGACACCCCAGCAGGTGGTCGCCATTGCCTCTAATG |
| | | GCGGCGGGAGACCCGCCTTGGAGAGCATTGTTGCCCAGTTATCTCGCCCTGAT |
| | | CCGGCGTTGGCCGCGTTGACCAACGACCACCTCGTCGCCTTGGCCTGCCTCGG |
| | | CGGGCGTCCTGCGCTGGATGCAGTGAAAAAGGGATTGGGGGATCCTATCAGCC |
| | | GTTCCCAGCTGGTGAAGTCCGAGCTGGAGGAGAAGAAATCCGAGTTGAGGCAC |
| | | AAGCTGAAGTACGTGCCCCACGAGTACATCGAGCTGATCGAGATCGCCCGGAA |
| | | CAGCACCCAGGACCGTATCCTGGAGATGAAGGTGATGGAGTTCTTCATGAAGG |
| | | TGTACGGCTACAGGGGCAAGCACCTGGGCGGCTCCAGGAAGCCCGACGGCGCC |
| | | ATCTACACCGTGGGCTCCCCCATCGACTACGGCGTGATCGTGGACACCAAGGC |
| | | CTACTCCGGCGGCTACAACCTGCCCATCGGCCAGGCCGACGAAATGCAGAGGT |
| | | ACGTGGAGGAGAACCAGACCAGGAACAAGCACATCAACCCCAACGAGTGGTGG |
| | | AAGGTGTACCCCTCCAGCGTGACCGAGTTCAAGTTCCTGTTCGTGTCCGGCCA |
| | | CTTCAAGGGCAACTACAAGGCCCAGCTGACCAGGCTGAACCACATCACCAACT |
| | | GCAACGGCGCCGTGCTGTCCGTGGAGGAGCTCCTGATCGGCGGCGAGATGATC |
| | | AAGGCCGGCACCCTGACCCTGGAGGAGGTGAGGAGGAAGTTCAACAACGGCGA |
| | | GATCAACTTCGCGGCCGACTGATAA |
| Left PD-1 | SEQ ID | MGDPKKKRKVIDIADLRTLGYSQQQQEKIKPKVRSTVAQHHEALVGHGFTHAH |
| KO TALEN | NO: 170 | IVALSQHPAALGTVAVKYQDMIAALPEATHEAIVGVGKQWSGARALEALLTVA |
| Amino acid | | GELRGPPLQLDTGQLLKIAKRGGVTAVEAVHAWRNALTGAPLNLTPEQVVAIA |
| sequence | | SKLGGKQALETVQALLPVLCQAHGLTPEQVVAIASHDGGKQALETVQRLLPVL |
| | | CQAHGLTPEQVVAIASHDGGKQALETVQRLLPVLCQAHGLTPQQVVAIASNGG |
| | | GKQALETVQRLLPVLCQAHGLTPEQVVAIASHDGGKQALETVQRLLPVLCQAH |
| | | GLTPQQVVAIASNGGGKQALETVQRLLPVLCQAHGLTPQQVVAIASYKGGKQA |
| | | LETVQRLLPVLCQAHGLTPQQVVAIASNGGGKQALETVQRLLPVLCQAHGLTP |
| | | QQVVAIASNNGGKQALETVQRLLPVLCQAHGLTPQQVVAIASNNGGKQALETV |
| | | QRLLPVLCQAHGLTPQQVVAIASNNGGKQALETVQRLLPVLCQAHGLTPQQVV |
| | | AIASNNGGKQALETVQRLLPVLCQAHGLTPEQVVAIASHDGGKQALETVQRLL |
| | | PVLCQAHGLTPEQVVAIASHDGGKQALETVQRLLPVLCQAHGLTPEQVVAIAS |
| | | NIGGKQALETVQALLPVLCQAHGLTPQQVVAIASNGGGRPALESIVAQLSRPD |
| | | PALAALTNDHLVALACLGGRPALDAVKKGLGDPISRSQLVKSELEEKKSELRH |
| | | KLKYVPHEYIELIEIARNSTQDRILEMKVMEFFMKVYGYRGKHLGGSRKPDGA |
| | | IYTVGSPIDYGVIVDTKAYSGGYNLPIGQADEMQRYVEENQTRNKHINPNEWW |
| | | KVYPSSVTEFKFLFVSGHFKGNYKAQLTRLNHITNCNGAVLSVEELLIGGEMI |
| | | KAGTLTLEEVRRKFNNGEINFAAD |
| Right PD-1 | SEQ ID | ATGGGCGATCCTAAAAAGAAACGTAAGGTCATCGATATCGCCGATCTACGCAC |
| KO TALEN | NO: 171 | GCTCGGCTACAGCCAGCAGCAACAGGAGAAGATCAAACCGAAGGTTCGTTCGA |
| mRNA | | CAGTGGCGCAGCACCACGAGGCACTGGTCGGCCACGGGTTTACACACGCGCAC |
| sequence | | ATCGTTGCGTTAAGCCAACACCCGGCAGCGTTAGGGACCGTCGCTGTCAAGTA |
| (pCLS25171) | | TCAGGACATGATCGCAGCGTTGCCAGAGGCGACACACGAAGCGATCGTTGGCG |
| | | TCGGCAAACAGTGGTCCGGCGCACGCGCTCTGGAGGCCTTGCTCACGGTGGCG |
| | | GGAGAGTTGAGAGGTCCACCGTTACAGTTGGACACAGGCCAACTTCTCAAGAT |
| | | TGCAAAACGTGGCGGCGTGACCGCAGTGGAGGCAGTGCATGCATGGCGCAATG |
| | | CACTGACGGGTGCCCCGCTCAACTTGACCCCCGAGCAAGTCGTCGCAATCGCC |
| | | AGCCATGATGGAGGGAAGCAAGCCCTCGAAACCGTGCAGCGGTTGCTTCCTGT |
| | | GCTCTGCCAGGCCCACGGCCTTACCCCTCAGCAGGTGGTGGCCATCGCAAGTA |
| | | ACGGAGGAGGAAAGCAAGCCTTGGAGACAGTGCAGCGCCTGTTGCCCGTGCTG |
| | | TGCCAGGCACACGGCCTCACACCAGAGCAGGTCGTGGCCATTGCCTCCCATGA |
| | | CGGGGGGAAACAGGCTCTGGAGACCGTCCAGAGGCTGCTGCCCGTCCTCTGTC |
| | | AAGCTCACGGCCTGACTCCCCAACAAGTGGTCGCCATCGCCTCTAATGGCGGC |
| | | GGGAAGCAGGCACTGGAAACAGTGCAGAGACTGCTCCCTGTGCTTTGCCAAGC |
| | | TCATGGGTTGACCCCCCAACAGGTCGTCGCTATTGCCTCAAACGGGGGGGGCA |
| | | AGCAGGCCCTTGAGACTGTGCAGAGGCTGTTGCCAGTGCTGTGTCAGGCTCAC |
| | | GGGCTCACTCCACAACAGGTGGTCGCAATTGCCAGCAACGGCGGCGGAAAGCA |
| | | AGCTCTTGAAACCGTGCAACGCCTCCTGCCCGTGCTCTGTCAGGCTCATGGCC |
| | | TGACACCACAACAAGTCGTGGCCATCGCCAGTAATAATGGCGGGAAACAGGCT |
| | | CTTGAGACCGTCCAGAGGCTGCTCCCAGTGCTCTGCCAGGCACACGGGCTGAC |
| | | CCCCGAGCAGGTGGTGGCTATCGCCAGCAATATTGGGGGCAAGCAGGCCCTGG |
| | | AAACAGTCCAGGCCCTGCTGCCAGTGCTTTGCCAGGCTCACGGGCTCACTCCC |
| | | CAGCAGGTCGTGGCAATCGCCTCCAACGGCGGAGGGAAGCAGGCTCTGGAGAC |
| | | CGTGCAGAGACTGCTGCCCGTCTTGTGCCAGGCCCACGGACTCACACCTGAAC |
| | | AGGTCGTCGCCATTGCCTCTCACGATGGGGGCAAACAAGCCCTGGAGACAGTG |
| | | CAGCGGCTGTTGCCTGTGTTGTGCCAAGCCCACGGCTTGACTCCTCAACAAGT |
| | | GGTCGCCATCGCCTCAAATGGCGGCGGAAAACAAGCTCTGGAGACAGTGCAGA |
| | | GGTTGCTGCCCGTCCTCTGCCAAGCCCACGGCCTGACTCCCCAACAGGTCGTC |
| | | GCCATTGCCAGCAACAACGGAGGAAAGCAGGCTCTCGAAACTGTGCAGCGGCT |
| | | GCTTCCTGTGCTGTGTCAGGCTCATGGGCTGACCCCCGAGCAAGTGGTGGCTA |
| | | TTGCCTCTAATGGAGGCAAGCAAGCCCTTGAGACAGTCCAGAGGCTGTTGCCA |
| | | GTGCTGTGCCAGGCCCACGGGCTCACACCCCAGCAGGTGGTCGCCATCGCCAG |
| | | TAACAACGGGGGCAAACAGGCATTGGAAACCGTCCAGCGCCTGCTTCCAGTGC |
| | | TCTGCCAGGCACACGGACTGACACCCGAACAGGTGGTGGCCATTGCATCCCAT |
| | | GATGGGGGCAAGCAGGCCCTGGAGACCGTGCAGAGACTCCTGCCAGTGTTGTG |
| | | CCAAGCTCACGGCCTCACCCCTCAGCAAGTCGTGGCCATCGCCTCAAACGGGG |
| | | GGGGCCGGCCTGCACTGGAGAGCATTGTTGCCCAGTTATCTCGCCCTGATCCG |
| | | GCGTTGGCCGCGTTGACCAACGACCACCTCGTCGCCTTGGCCTGCCTCGGCGG |
| | | GCGTCCTGCGCTGGATGCAGTGAAAAAGGGATTGGGGGATCCTATCAGCCGTT |
| | | CCCAGCTGGTGAAGTCCGAGCTGGAGGAGAAGAAATCCGAGTTGAGGCACAAG |
| | | CTGAAGTACGTGCCCCACGAGTACATCGAGCTGATCGAGATCGCCCGGAACAG |
| | | CACCCAGGACCGTATCCTGGAGATGAAGGTGATGGAGTTCTTCATGAAGGTGT |
| | | ACGGCTACAGGGGCAAGCACCTGGGCGGCTCCAGGAAGCCCGACGGCGCCATC |
| | | TACACCGTGGGCTCCCCCATCGACTACGGCGTGATCGTGGACACCAAGGCCTA |
| | | CTCCGGCGGCTACAACCTGCCCATCGGCCAGGCCGACGAAATGCAGAGGTACG |
| | | TGGAGGAGAACCAGACCAGGAACAAGCACATCAACCCCAACGAGTGGTGGAAG |
| | | GTGTACCCCTCCAGCGTGACCGAGTTCAAGTTCCTGTTCGTGTCCGGCCACTT |
| | | CAAGGGCAACTACAAGGCCCAGCTGACCAGGCTGAACCACATCACCAACTGCA |
| | | ACGGCGCCGTGCTGTCCGTGGAGGAGCTCCTGATCGGCGGCGAGATGATCAAG |
| | | GCCGGCACCCTGACCCTGGAGGAGGTGAGGAGGAAGTTCAACAACGGCGAGAT |
| | | CAACTTCGCGGCCGACTGATAA |
| Right PD-1 | SEQ ID | MGDPKKKRKVIDIADLRTLGYSQQQQEKIKPKVRSTVAQHHEALVGHGFTHAH |
| KO TALEN | NO: 172 | IVALSQHPAALGTVAVKYQDMIAALPEATHEAIVGVGKQWSGARALEALLTVA |
| pCLS34486 | | GELRGPPLQLDTGQLLKIAKRGGVTAVEAVHAWRNALTGAPLNLTPEQVVAIA |
| CISHe3_9- | | SHDGGKQALETVQRLLPVLCQAHGLTPQQVVAIASNGGGKQALETVQRLLPVL |
| R1 | | CQAHGLTPEQVVAIASHDGGKQALETVQRLLPVLCQAHGLTPQQVVAIASNGG |
| Amino acid | | GKQALETVQRLLPVLCQAHGLTPQQVVAIASNGGGKQALETVQRLLPVLCQAH |
| sequence | | GLTPQQVVAIASNGGGKQALETVQRLLPVLCQAHGLTPQQVVAIASNNGGKQA |
| | | LETVQRLLPVLCQAHGLTPEQVVAIASNIGGKQALETVQALLPVLCQAHGLTP |
| | | QQVVAIASNGGGKQALETVQRLLPVLCQAHGLTPEQVVAIASHDGGKQALETV |
| | | QRLLPVLCQAHGLTPQQVVAIASNGGGKQALETVQRLLPVLCQAHGLTPQQVV |
| | | AIASNNGGKQALETVQRLLPVLCQAHGLTPEQVVAIASNGGKQALETVQRLLP |
| | | VLCQAHGLTPQQVVAIASNNGGKQALETVQRLLPVLCQAHGLTPEQVVAIASH |
| | | DGGKQALETVQRLLPVLCQAHGLTPQQVVAIASNGGGRPALESIVAQLSRPDP |
| | | ALAALTNDHLVALACLGGRPALDAVKKGLGDPISRSQLVKSELEEKKSELRHK |
| | | LKYVPHEYIELIEIARNSTQDRILEMKVMEFFMKVYGYRGKHLGGSRKPDGAI |
| | | YTVGSPIDYGVIVDTKAYSGGYNLPIGQADEMQRYVEENQTRNKHINPNEWWK |
| | | VYPSSVTEFKFLFVSGHFKGNYKAQLTRLNHITNCNGAVLSVEELLIGGEMIK |
| | | AGTLTLEEVRRKFNNGEINFAAD |

### Experimental Results

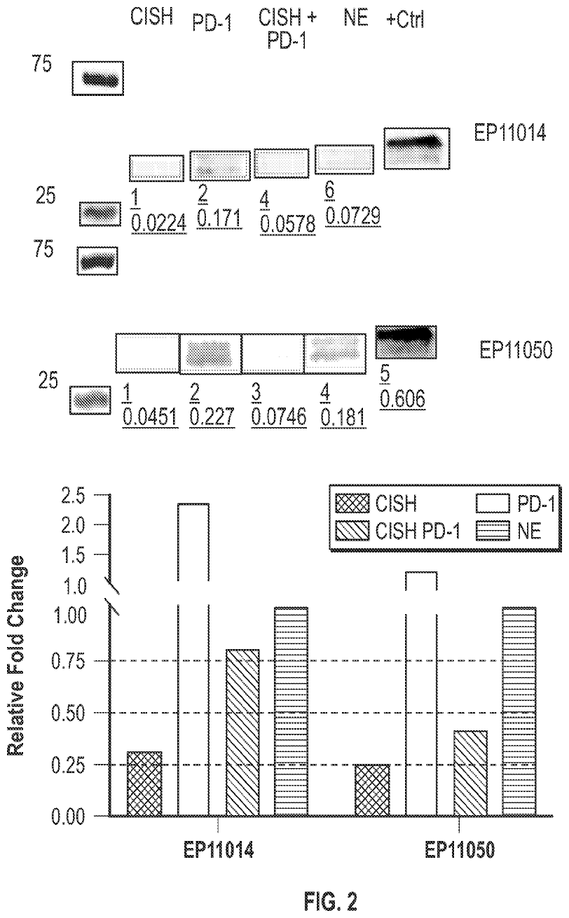

The efficiency of single and double CISH KO was 75% and 40%, respectively (FIG. 2). Post-Expansion TILs were re-stimulated with anti-CD3 overnight prior to be assessed for CISH protein by western blot analysis. NE=non-electroporation; +Ctrl=293T cells overexpressing CISH proteins; Densitometry data from western blot analysis were used for calculation of relative fold change; NE was used for baseline calculation.

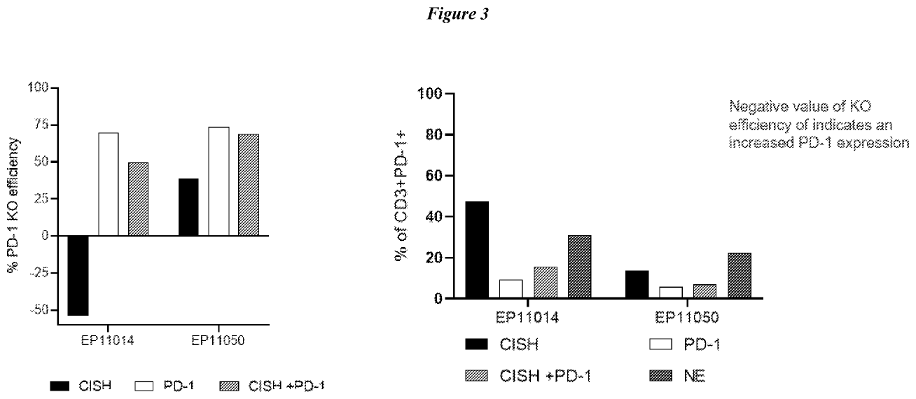

PD-1 KO efficiency ranged from 50 to 75% in double CISH/PD-1 KO TIL (FIG. 3). Post-Expansion TILs were re-stimulated with anti-CD3 overnight prior to be assessed for PD-1 expression by flow cytometry. Negative value of KO efficiency of indicates an increased PD-1 expression.

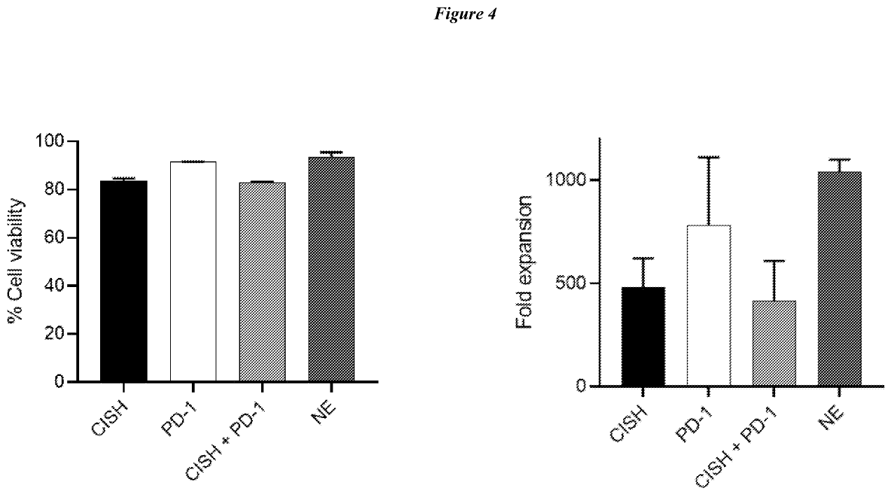

Fold expansion in CISH KO TIL decreased relative to control (FIG. 4). Post-Expansion TILs were counted and assessed for cell viability. Fold expansion was calculated by the total cell count of post-Expansion TILs divided by the number cells seeded on Day 0 of the expansion.

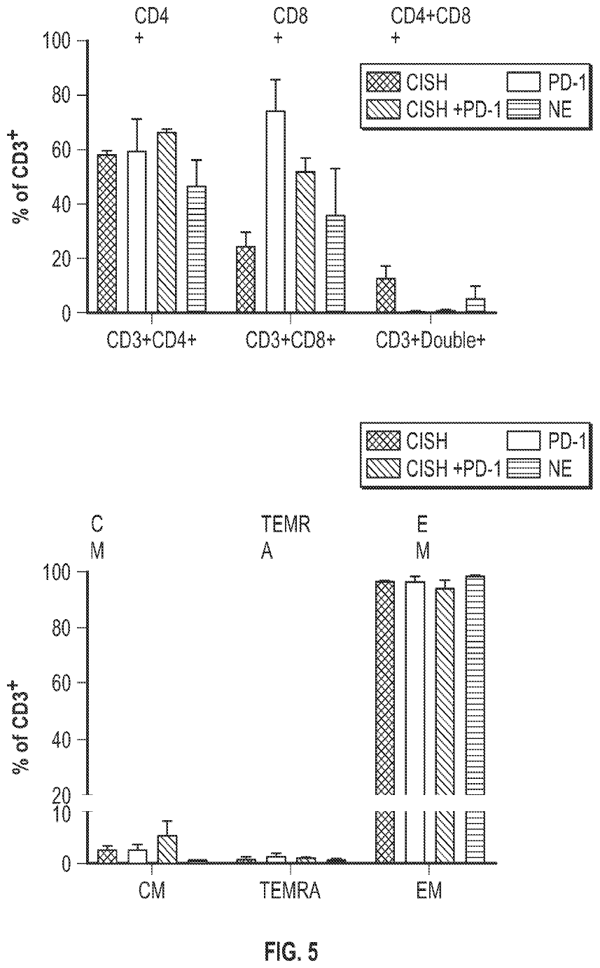

The phenotype of CISH KO TIL in terms of T-cell Lineage and Memory Subset was comparable to non-electroporated control (FIG. 5). Post-Expansion TILs were stained for CD3, CD4, CD8, CD45RA and CCR7. The cells were acquired on BD FACSCanto™ and analyzed by FlowJo.

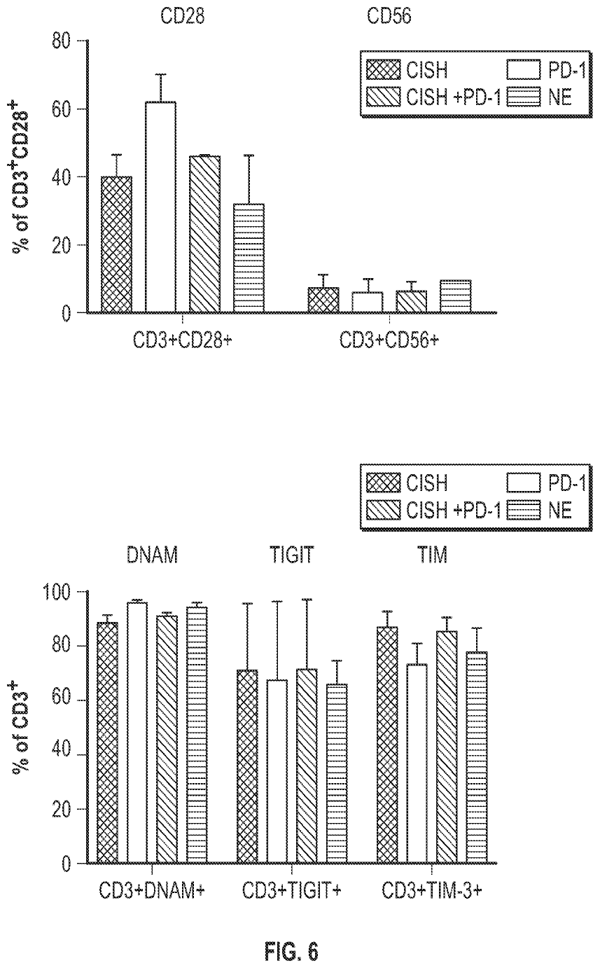

The phenotype of CISH KO TIL in terms of differentiation and activation/exhaustion was comparable to non-electroporated control (FIG. 6). Post-Expansion TILs were stained for CD3, CD28, CD56, DNAM, TIGIT, and TIM-3. The cells were acquired on BD FACSCanto™ and analyzed by FlowJo.

### Example 2: CISH Knock-Out Efficiency

### Experimental Design

Genomic DNAs isolated from nine pairs of CISH knockout and non-electroporated TIL were amplified with forward and reverse primers (CISH-F1 and CISH-RI) using PCR.

PCR products were analyzed by NGS sequencing.

Data analysis was performed using CRISPresso 2.

### Primers, Cleavage Site, and CISH Sequence

CISH forward primer
(SEQ ID NO: 173)
CTGCACTGCTGATACCCGAA
CISH reverse primer
(SEQ ID NO: 174)
GGGGTACTGTCGGAGGTAGT
Cleavage site:
(SEQ ID NO: 168)
TGCGCCTAGTGACCCAGCACTGCCTGCTCCTCCACCAGCCACTGCTGTA
CISH target site Sequence:
(SEQ ID NO: 175)
CTGCACTGCTGATACCCGAAGCGACAGCCCCGATCCTGCTCCCACCCCG
GCCCTGCCTATGCCTAAGGAGGATGCGCCTAGTGACCCAGCACTGCCTG
CTCCTCCACCAGCCACTGCTGTACACCTAAAACTGGTGCAGCCCTTTGT
ACGCAGAAGCAGTGCCCGCAGCCTGCAACACCTGTGCCGCCTTGTCATC
AACCGTCTGGTGGCCGACGTGGACTGCCTGCCACTGCCCCGGCGCATGG
CCGACTACCTCCGACAGTACCC.
Three underlined regions correspond to the
specific locations on the CISH sequence.

TABLE 9
Results - CISH KO efficiency.

| TIL ID | Histology | % KO efficiency |
|---|---|---|
| EP11014 | Breast | 80.67 |
| EP11050 | Breast | 78.62 |
| EP11119 | Breast | 46.38 |
| H3123 | Head and neck | 35.04 |
| H3125 | Head and neck | 31.93 |
| H3139 | Head and neck | 83.14 |
| H3142 | Head and neck | 87.10 |
| M1152 | Melanoma | 87.40 |
| OV8154 | Ovarian | 88.07 |

| | |
|---|---|
| Average | 68.71277863 |

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A method of preparing genetically modified tumor infiltrating lymphocytes (TILs) comprising reduced expression of CISH, the method comprising:
(a) introducing into the TILs nucleic acid(s) encoding one or more first Transcription activator-like effector nucleases (TALE-nuclease) able to selectively inactivate by DNA cleavage a gene encoding CISH; and
(b) expanding the TILs.

2. The method according to claim 1, wherein introducing into the TILs nucleic acid(s) encoding the one or more first TALE-nucleases comprises an electroporation step.

3. The method according to claim 1, wherein the nucleic acid(s) encoding the one or more first TALE-nucleases are RNA and said RNA are introduced into the TILs by electroporation.

4. The method according to claim 1, wherein the method further comprises prior to the introducing step, a step of activating TILs by culturing the TILs in a cell culture medium in the presence of OKT-3 for about 1-3 days.

5. The method according to claim 1, wherein the method further comprises after the introducing step and before the expanding step, a step of resting the TILs in a cell culture medium comprising IL-2 for about 1 day.

6. The method according to claim 1, wherein the method further comprises prior to the introducing step, a step of cryopreserving the TILs followed by thawing and culturing the TILs in a cell culture medium comprising IL-2 for about 1-3 days.

7. (canceled)

8. The method according to claim 1, wherein the one or more first TALE-nucleases are each constituted by a first half-TALE nuclease and a second half-TALE nuclease.

9. The method according to claim 8, wherein the first half-TALE nuclease is a first fusion protein constituted by a first TALE nucleic acid binding domain fused to a first nuclease catalytic domain and the second half-TALE nuclease is a second fusion protein constituted by a second TALE nucleic acid binding domain fused to a second nuclease catalytic domain.

10. The method according to claim 9, wherein the first TALE nucleic acid binding domain has a first amino acid sequence and the second TALE nucleic acid binding domain has a second amino acid sequence, and wherein the first amino acid sequence is different from the second amino acid sequence.

11. The method according to claim 9, wherein the first nuclease catalytic domain has a first amino acid sequence and the second nuclease catalytic domain has a second amino acid sequence, and wherein the first amino acid sequence is the same as the second amino acid sequence.

12. The method according to claim 9, wherein the first nuclease catalytic domain and the second nuclease catalytic domain both have the amino acid sequence of Fok-I.

13. The method according to claim 8, wherein the first half-TALE nuclease and the second half-TALE nuclease are capable of forming a heterodimeric DNA cleavage complex to effect DNA cleavage at the target site in the gene encoding CISH, and wherein the target site in the gene encoding CISH comprises the nucleic acid sequence of SEQ ID NO: 175.

14. The method according to claim 8, wherein the first half-TALE nuclease recognizes a first half-target located at a first location in the target site in the gene encoding CISH and the second half-TALE nuclease recognizes a second half-target located in a second location in the target site in the gene encoding CISH that does not overlap with the first location.

15. The method according to claim 1, wherein the TALE nuclease comprises an amino acid sequence having at least 90% sequence identity with an amino acid sequence selected from the group consisting of SEQ ID NO: 165 and SEQ ID NO: 167.

16. (canceled)

17. The method according to claim 8, wherein said first half-TALE-nuclease comprises the amino acid sequence having at least 90% sequence identity with SEQ ID NO: 165 and said second half-TALE-nuclease comprises the amino acid sequence having at least 70%, 75%, 80%, 85%, 87.5%, 90%, 92.5%, 95%, 97.5%, 98%, or 99% sequence identity with SEQ ID NO: 167.

18. (canceled)

19. The method according to claim 1, wherein the expanded TILs comprise sufficient TILs for administering a therapeutically effective dosage of the TILs to a subject in need thereof.

20. The method according to claim 19, wherein the therapeutically effective dosage of the expanded TILs comprises from about 1×10⁹ to about 9×10¹⁰ TILs.

21. (canceled)

22. A Transcription activator-like effector nuclease (TALE-nuclease) that recognizes and effects DNA cleavage at a target site in a gene encoding CISH, wherein the TALE-nuclease comprises an amino acid sequence having at least 90% sequence identity with an amino acid sequence selected from the group consisting of SEQ ID NO: 165 and SEQ ID NO: 167.

23.-31. (canceled)

32. A method for expanding genetically modified tumor infiltrating lymphocytes (TILs) into a therapeutic population of TILs comprising reduced expression of CISH, the method comprising:
(a) obtaining and/or receiving a first population of TILs from a tumor resected from a subject by processing a tumor sample obtained from the subject into multiple tumor fragments;
(b) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs;
(c) introducing into the TILs nucleic acid(s) encoding one or more first Transcription activator-like effector nucleases (TALE-nuclease) able to selectively inactivate by DNA cleavage a gene encoding CISH, wherein the one or more first TALE-nucleases comprise a TALE-nuclease that is directed against a target site in the gene encoding CISH, wherein the target site comprises the nucleic acid sequence of SEQ ID NO: 175;
(d) performing a second expansion by culturing the TILs obtained from step (c) in a cell culture medium comprising IL-2, OKT-3, and antigen presenting cells (APCs), to produce a third population of TILs, wherein the second expansion is performed for about 7-14 days to obtain the third population of TILs, wherein the third population of TILs is a therapeutic population of TILs.

33-74. (canceled)

75. A method of treating cancer in a subject in need thereof, the method comprising administering to the subject a therapeutic population of genetically modified tumor infiltrating lymphocytes (TILs) comprising reduced expression of CISH and/or CISH and PD-1, wherein the therapeutic population of genetically modified TILs is obtainable by the method of claim 1.

76. (canceled)


SEQUENCE LISTING (175 sequences)

SEQ ID NO: 1 - protein, 450 aa, Artificial Sequence
  Muromonab heavy chain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  Muromonab light chain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 134 aa, Artificial Sequence
  recombinant human IL-2 (rhIL-2)
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - protein, 132 aa, Artificial Sequence
  Aldesleukin
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 130 aa, Artificial Sequence
  recombinant human IL-4 (rhIL-4)
MHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 6 - protein, 153 aa, Artificial Sequence
  recombinant human IL-7 (rhIL-7)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 7 - protein, 115 aa, Artificial Sequence
  recombinant human IL-15 (rhIL-15)
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - protein, 132 aa, Artificial Sequence
  recombinant human IL-21 (rhIL-21)
MQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 9-126 - skipped (118 entries)

SEQ ID NO: 127 - DNA, 49 bp, Artificial Sequence
  Target PD-1 Sequence
ttctccccagccctgctcgtggtgaccgaaggggacaacgccaccttca

SEQ ID NO: 128 - DNA, 49 bp, Artificial Sequence
  Target PD-1 Sequence
tacctctgtggggccatctccctggcccccaaggcgcagatcaaagaga

SEQ ID NO: 129 - protein, 530 aa, Artificial Sequence
  530 PRT Artificial Sequence synthetic construct Repeat PD-1-left
LTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALE

SEQ ID NO: 130 - protein, 529 aa, Artificial Sequence
  529 PRT Artificial Sequence synthetic construct Repeat PD-1-right
LTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGRPALE

SEQ ID NO: 131 - protein, 530 aa, Artificial Sequence
  530 PRT Artificial Sequence synthetic construct Repeat PD-1-left
LTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALE

SEQ ID NO: 132 - protein, 529 aa, Artificial Sequence
  529 PRT Artificial Sequence synthetic construct Repeat PD-1-right
LTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALE

SEQ ID NO: 133 - DNA, 2814 bp, Artificial Sequence
  2814 DNA Artificial Sequence synthetic construct PD-1-left TALEN 133
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 134 - DNA, 2829 bp, Artificial Sequence
  2829 DNA Artificial Sequence synthetic construct PD-1-right TALEN 134
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaagtcgtcgcaatcgccagcaataacggagggaagcaagccctcgaaaccgtgcagcggttgcttcctgtgctctgccaggcccacggccttacccctgagcaggtggtggccatcgcaagtaacattggaggaaagcaagccttggagacagtgcaggccctgttgcccgtgctgtgccaggcacacggcctcacaccagagcaggtcgtggccattgcctccaacatcggggggaaacaggctctggagaccgtccaggccctgctgcccgtcctctgtcaagctcacggcctgactccccaacaagtggtcgccatcgcctctaataacggcgggaagcaggcactggaaacagtgcagagactgctccctgtgctttgccaagctcatgggttgaccccccaacaggtcgtcgctattgcctcaaacaacgggggcaagcaggcccttgagactgtgcagaggctgttgccagtgctgtgtcaggctcacgggctcactccacaacaggtggtcgcaattgccagcaacggcggcggaaagcaagctcttgaaaccgtgcaacgcctcctgcccgtgctctgtcaggctcatggcctgacaccacaacaagtcgtggccatcgccagtaataatggcgggaaacaggctcttgagaccgtccagaggctgctcccagtgctctgccaggcacacgggctgaccccccagcaggtggtggctatcgccagcaataatgggggcaagcaggccctggaaacagtccagcgcctgctgccagtgctttgccaggctcacgggctcactcccgaacaggtcgtggcaatcgcctccaacggagggaagcaggctctggagaccgtgcagagactgctgcccgtcttgtgccaggcccacggactcacacctcagcaggtcgtcgccattgcctctaacaacgggggcaaacaagccctggagacagtgcagcggctgttgcctgtgttgtgccaagcccacggcttgactcctcaacaagtggtcgccatcgcctcaaatggcggcggaaaacaagctctggagacagtgcagaggttgctgcccgtcctctgccaagcccacggcctgactccccaacaggtcgtcgccattgccagcaacggcggaggaaagcaggctctcgaaactgtgcagcggctgcttcctgtgctgtgtcaggctcatgggctgaccccccagcaagtggtggctattgcctctaacaatggaggcaagcaagcccttgagacagtccagaggctgttgccagtgctgtgccaggcccacgggctcacaccccagcaggtggtcgccatcgccagtaacggcgggggcaaacaggcattggaaaccgtccagcgcctgcttccagtgctctgccaggcacacggactgacacccgaacaggtggtggccattgcatcccatgatgggggcaagcaggccctggagaccgtgcagagactcctgccagtgttgtgccaagctcacggcctcacccctcagcaagtcgtggccatcgcctcaaacggggggggccggcctgcactggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 135 - DNA, 2814 bp, Artificial Sequence
  2814 DNA Artificial Sequence synthetic construct PD-1-left TALEN 135
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 136 - DNA, 2829 bp, Artificial Sequence
  2829 DNA Artificial Sequence synthetic construct PD-1-right TALEN 136
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgagcaagtcgtcgcaatcgccagccatgatggagggaagcaagccctcgaaaccgtgcagcggttgcttcctgtgctctgccaggcccacggccttacccctcagcaggtggtggccatcgcaagtaacggaggaggaaagcaagccttggagacagtgcagcgcctgttgcccgtgctgtgccaggcacacggcctcacaccagagcaggtcgtggccattgcctcccatgacggggggaaacaggctctggagaccgtccagaggctgctgcccgtcctctgtcaagctcacggcctgactccccaacaagtggtcgccatcgcctctaatggcggcgggaagcaggcactggaaacagtgcagagactgctccctgtgctttgccaagctcatgggttgaccccccaacaggtcgtcgctattgcctcaaacggggggggcaagcaggcccttgagactgtgcagaggctgttgccagtgctgtgtcaggctcacgggctcactccacaacaggtggtcgcaattgccagcaacggcggcggaaagcaagctcttgaaaccgtgcaacgcctcctgcccgtgctctgtcaggctcatggcctgacaccacaacaagtcgtggccatcgccagtaataatggcgggaaacaggctcttgagaccgtccagaggctgctcccagtgctctgccaggcacacgggctgacccccgagcaggtggtggctatcgccagcaatattgggggcaagcaggccctggaaacagtccaggccctgctgccagtgctttgccaggctcacgggctcactccccagcaggtcgtggcaatcgcctccaacggcggagggaagcaggctctggagaccgtgcagagactgctgcccgtcttgtgccaggcccacggactcacacctgaacaggtcgtcgccattgcctctcacgatgggggcaaacaagccctggagacagtgcagcggctgttgcctgtgttgtgccaagcccacggcttgactcctcaacaagtggtcgccatcgcctcaaatggcggcggaaaacaagctctggagacagtgcagaggttgctgcccgtcctctgccaagcccacggcctgactccccaacaggtcgtcgccattgccagcaacaacggaggaaagcaggctctcgaaactgtgcagcggctgcttcctgtgctgtgtcaggctcatgggctgacccccgagcaagtggtggctattgcctctaatggaggcaagcaagcccttgagacagtccagaggctgttgccagtgctgtgccaggcccacgggctcacaccccagcaggtggtcgccatcgccagtaacaacgggggcaaacaggcattggaaaccgtccagcgcctgcttccagtgctctgccaggcacacggactgacacccgaacaggtggtggccattgcatcccatgatgggggcaagcaggccctggagaccgtgcagagactcctgccagtgttgtgccaagctcacggcctcacccctcagcaagtcgtggccatcgcctcaaacggggggggccggcctgcactggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 137 - protein, 153 aa, Artificial Sequence
  IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 138 - protein, 133 aa, Artificial Sequence
  IL-2 mutein
APTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 139 - protein, 133 aa, Artificial Sequence
  IL-2 mutein
APTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 140 - protein, 145 aa, Artificial Sequence
  HCDR1_IL-2
GFSLAPTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGMSVG

SEQ ID NO: 141 - protein, 16 aa, Artificial Sequence
  HCDR2
DIWWDDKKDYNPSLKS

SEQ ID NO: 142 - protein, 10 aa, Artificial Sequence
  HCDR3
SMITNWYFDV

SEQ ID NO: 143 - protein, 91 aa, Artificial Sequence
  HCDR1_IL-2 kabat
TELKHLQCLEEELKPLEEVLNLAQSKNFHLRPRDLISNINVIVLELKGSETTFMCEYADETATIVEFLNRWITFCQSIISTLTSTSGMSVG

SEQ ID NO: 144 - protein, 16 aa, Artificial Sequence
  HCDR2 kabat
DIWWDDKKDYNPSLKS

SEQ ID NO: 145 - protein, 10 aa, Artificial Sequence
  HCDR3 kabat
SMITNWYFDV

SEQ ID NO: 146 - protein, 142 aa, Artificial Sequence
  HCDR1_IL-2 clothia
GFSLAPTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGM

SEQ ID NO: 147 - protein, 5 aa, Artificial Sequence
  HCDR2 clothia
WWDDK

SEQ ID NO: 148 - protein, 10 aa, Artificial Sequence
  HCDR3 clothia
SMITNWYFDV

SEQ ID NO: 149 - protein, 143 aa, Artificial Sequence
  HCDR1_IL-2 IMGT
GFSLAPTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGMS

SEQ ID NO: 150 - protein, 7 aa, Artificial Sequence
  HCDR2 IMGT
IWWDDKK

SEQ ID NO: 151 - protein, 12 aa, Artificial Sequence
  HCDR3 IMGT
ARSMITNWYFDV

SEQ ID NO: 152 - protein, 253 aa, Artificial Sequence
  VH
QVTLRESGPALVKPTQTLTLTCTFSGFSLAPTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGMSVGWIRQPPGKALEWLADIWWDDKKDYNPSLKSRLTISKDTSKNQVVLKVTNMDPADTATYYCARSMITNWYFDVWGAGTTVTVSS

SEQ ID NO: 153 - protein, 533 aa, Artificial Sequence
  Heavy chain
QMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 10 aa, Artificial Sequence
  LCDR1 kabat
KAQLSVGYMH

SEQ ID NO: 155 - protein, 7 aa, Artificial Sequence
  LCDR2 kabat
DTSKLAS

SEQ ID NO: 156 - protein, 9 aa, Artificial Sequence
  LCDR3 kabat
FQGSGYPFT

SEQ ID NO: 157 - protein, 6 aa, Artificial Sequence
  LCDR1 chothia
QLSVGY

SEQ ID NO: 158 - protein, 3 aa, Artificial Sequence
  LCDR2 chothia
DTS

SEQ ID NO: 159 - protein, 6 aa, Artificial Sequence
  LCDR3 chothia
GSGYPF

SEQ ID NO: 160 - protein, 106 aa, Artificial Sequence
  VL
DIQMTQSPSTLSASVGDRVTITCKAQLSVGYMHWYQQKPGKAPKLLIYDTSKLASGVPSRFSGSGSGTEFTLTISSLQPDDFATYYCFQGSGYPFTFGGGTKLEIK

SEQ ID NO: 161 - protein, 213 aa, Artificial Sequence
  Light chain
DIQMTQSPSTLSASVGDRVTITCKAQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 583 aa, Artificial Sequence
  Light chain
QVTLRESGPALVKPTQTLTLTCTFSGFSLAPTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 213 aa, Artificial Sequence; identical to SEQ ID NO: 161
  Light chain

SEQ ID NO: 164 - DNA, 2781 bp, Artificial Sequence
  Left CISH KO TALEN pCLS34485 CISHe3_9-L1 Nucleotide sequence
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 165 - protein, 925 aa, Artificial Sequence
  Left CISH KO TALEN pCLS34485 CISHe3_9-L1 Amino acid sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 166 - DNA, 2781 bp, Artificial Sequence
  Right CISH KO TALEN pCLS34486 CISHe3_9-R1 Nucleotide sequence
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 167 - protein, 925 aa, Artificial Sequence
  Right CISH KO TALEN pCLS34486 CISHe3_9-R1 Amino acid sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 168 - DNA, 49 bp, Artificial Sequence
  CISH KO Cleavage Site sequence CISHe3_9.1 Nucleotide sequence
tgcgcctagtgacccagcactgcctgctcctccaccagccactgctgta

SEQ ID NO: 169 - DNA, 2781 bp, Artificial Sequence
  Left PD-1 KO TALEN mRNA sequence (pCLS29259)
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgagcaagtggtggctatcgcttccaagctggggggaaagcaggccctggagaccgtccaggcccttctcccagtgctttgccaggctcacggactgacccctgaacaggtggtggcaattgcctcacacgacgggggcaagcaggcactggagactgtccagcggctgctgcctgtcctctgccaggcccacggactcactcctgagcaggtcgtggccattgccagccacgatgggggcaaacaggctctggagaccgtgcagcgcctcctcccagtgctgtgccaggctcatgggctgaccccacagcaggtcgtcgccattgccagtaacggcggggggaagcaggccctcgaaacagtgcagaggctgctgcccgtcttgtgccaagcacacggcctgacacccgagcaggtggtggccatcgcctctcatgacggcggcaagcaggcccttgagacagtgcagagactgttgcccgtgttgtgtcaggcccacgggttgacaccccagcaggtggtcgccatcgccagcaatggcgggggaaagcaggcccttgagaccgtgcagcggttgcttccagtgttgtgccaggcacacggactgacccctcaacaggtggtcgcaatcgccagctacaagggcggaaagcaggctctggagacagtgcagcgcctcctgcccgtgctgtgtcaggctcacggactgacaccacagcaggtggtcgccatcgccagtaacgggggcggcaagcaggctttggagaccgtccagagactcctccccgtcctttgccaggcccacgggttgacacctcagcaggtcgtcgccattgcctccaacaacgggggcaagcaggccctcgaaactgtgcagaggctgctgcctgtgctgtgccaggctcatgggctgacaccccagcaggtggtggccattgcctctaacaacggcggcaaacaggcactggagaccgtgcaaaggctgctgcccgtcctctgccaagcccacgggctcactccacagcaggtcgtggccatcgcctcaaacaatggcgggaagcaggccctggagactgtgcaaaggctgctccctgtgctctgccaggcacacggactgacccctcagcaggtggtggcaatcgcttccaacaacgggggaaagcaggccctcgaaaccgtgcagcgcctcctcccagtgctgtgccaggcacatggcctcacacccgagcaagtggtggctatcgccagccacgacggagggaagcaggctctggagaccgtgcagaggctgctgcctgtcctgtgccaggcccacgggcttactccagagcaggtcgtcgccatcgccagtcatgatggggggaagcaggcccttgagacagtccagcggctgctgccagtcctttgccaggctcacggcttgactcccgagcaggtcgtggccattgcctcaaacattgggggcaaacaggccctggagacagtgcaggccctgctgcccgtgttgtgtcaggcccacggcttgacaccccagcaggtggtcgccattgcctctaatggcggcgggagacccgccttggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 170 - protein, 925 aa, Artificial Sequence
  Left PD-1 KO TALEN Amino acid sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASKLGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASYKGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 171 - DNA, 2778 bp, Artificial Sequence
  Right PD-1 KO TALEN mRNA sequence (pCLS25171)
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgagcaagtcgtcgcaatcgccagccatgatggagggaagcaagccctcgaaaccgtgcagcggttgcttcctgtgctctgccaggcccacggccttacccctcagcaggtggtggccatcgcaagtaacggaggaggaaagcaagccttggagacagtgcagcgcctgttgcccgtgctgtgccaggcacacggcctcacaccagagcaggtcgtggccattgcctcccatgacggggggaaacaggctctggagaccgtccagaggctgctgcccgtcctctgtcaagctcacggcctgactccccaacaagtggtcgccatcgcctctaatggcggcgggaagcaggcactggaaacagtgcagagactgctccctgtgctttgccaagctcatgggttgaccccccaacaggtcgtcgctattgcctcaaacggggggggcaagcaggcccttgagactgtgcagaggctgttgccagtgctgtgtcaggctcacgggctcactccacaacaggtggtcgcaattgccagcaacggcggcggaaagcaagctcttgaaaccgtgcaacgcctcctgcccgtgctctgtcaggctcatggcctgacaccacaacaagtcgtggccatcgccagtaataatggcgggaaacaggctcttgagaccgtccagaggctgctcccagtgctctgccaggcacacgggctgacccccgagcaggtggtggctatcgccagcaatattgggggcaagcaggccctggaaacagtccaggccctgctgccagtgctttgccaggctcacgggctcactccccagcaggtcgtggcaatcgcctccaacggcggagggaagcaggctctggagaccgtgcagagactgctgcccgtcttgtgccaggcccacggactcacacctgaacaggtcgtcgccattgcctctcacgatgggggcaaacaagccctggagacagtgcagcggctgttgcctgtgttgtgccaagcccacggcttgactcctcaacaagtggtcgccatcgcctcaaatggcggcggaaaacaagctctggagacagtgcagaggttgctgcccgtcctctgccaagcccacggcctgactccccaacaggtcgtcgccattgccagcaacaacggaggaaagcaggctctcgaaactgtgcagcggctgcttcctgtgctgtgtcaggctcatgggctgacccccgagcaagtggtggctattgcctctaatggaggcaagcaagcccttgagacagtccagaggctgttgccagtgctgtgccaggcccacgggctcacaccccagcaggtggtcgccatcgccagtaacaacgggggcaaacaggcattggaaaccgtccagcgcctgcttccagtgctctgccaggcacacggactgacacccgaacaggtggtggccattgcatcccatgatgggggcaagcaggccctggagaccgtgcagagactcctgccagtgttgtgccaagctcacggcctcacccctcagcaagtcgtggccatcgcctcaaacggggggggccggcctgcactggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 172 - protein, 924 aa, Artificial Sequence
  pCLS34486 CISHe3_9- R1 Amino acid sequence
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 173 - DNA, 20 bp, Artificial Sequence
  CISH forward primer
ctgcactgctgatacccgaa

SEQ ID NO: 174 - DNA, 20 bp, Artificial Sequence
  CISH reverse primer
ggggtactgtcggaggtagt

SEQ ID NO: 175 - DNA, 267 bp, Artificial Sequence
  CISH target site Sequence
ctgcactgctgatacccgaagcgacagccccgatcctgctcccaccccggccctgcctatgcctaaggaggatgcgcctagtgacccagcactgcctgctcctccaccagccactgctgtacacctaaaactggtgcagccctttgtacgcagaagcagtgcccgcagcctgcaacacctgtgccgccttgtcatcaaccgtctggtggccgacgtggactgcctgccactgccccggcgcatggccgactacctccgacagtaccc